(19) United States
(12) Patent Application Publication
(10) Pub. No.: US 2022/0177862 A1
(43) Pub. Date: Jun. 9, 2022

(54) Precise Gene Activation Via Novel Designed Proteins Mediating Epigenetic Remodeling

(71) Applicant: UNIVERSITY OF WASHINGTON, Seattle, WA (US)

(72) Inventors: Shiri LEVY, Seattle, WA (US); Hannele RUOHOLA-BAKER, Seattle, WA (US); David BAKER, Seattle, WA (US)

(21) Appl. No.: 17/434,832

(22) PCT Filed: Mar. 11, 2020
(86) PCT No.: PCT/US2020/022014

§ 371 Date: Aug. 30, 2021

Related U.S. Application Data
(60) Provisional application No. 62/866,295, filed on Jun. 25, 2019.
(60) Provisional application No. 62/817,189, filed on Mar. 12, 2019.

(51) Int. Cl. C12N 9/22; C12N 15/11; C12N 15/90

(52) U.S. Cl. CPC C12N 9/22; C12N 15/11; C12N 15/907; A61K 38/00; C12N 2800/80; C12N 2310/20; C07K 2319/01

(57) ABSTRACT

Disclosed herein are compositions including an embryonic ectoderm development (BED) polypeptide binder (EB) domain and a CRISPR associated protein 9 (CAS9) domain linked to the EB domain, and uses thereof for gene activation in a biological cell.

### CROSS REFERENCE

This application claims priority to U.S. Provisional Patent Application Ser. No. 62/817,189 filed Mar. 12, 2019 and 62/866,295 filed Jun. 25, 2019, each incorporated by reference herein in its entirety.

### FEDERAL FUNDING STATEMENT

This invention was made with government support under Grant No. GM081619, awarded by the National Institutes of Health. The government has certain rights in the invention.

### REFERENCE TO SEQUENCE LISTING

This application contains a Sequence Listing submitted as an electronic text file named “19-139-PCT Sequence-Listing_ST25.txt”, having a size in bytes of 275 kb, and created on Mar. 7, 2020. The information contained in this electronic file is hereby incorporated by reference in its entirety pursuant to 37 CFR § 1.52(e)(5).

### BACKGROUND

A central problem in epigenetics and developmental biology is the role of specific histone 3 lysine 27 methylation (H3K27me3) marks in cell fate decisions. PRC2 is an evolutionarily conserved H3K27me3 methyltransferase complex that plays a key role in developmental transitions by repressing many genes, but it is not known which, if any, H3K27me3 marks at specific loci are required for gene regulation and cell fate determination. There is no current way to inhibit PRC2 function precisely at specific genetic loci, and hence to target demethylation of histone3 K27 which play key roles in the repression of transcription.

### SUMMARY

In one aspect, the disclosure provides compositions, comprising:

(a) an embryonic ectoderm development (EED) polypeptide binder (EB) domain; and

(b) a CRISPR associated protein 9 (CAS9) domain linked to the EB domain. In one embodiment the EB domain and the CAS9 domain are expressed in a fusion protein, and may be separated by an amino acid linker connecting the EB domain and the CAS domain. In another embodiment, the EB domain comprises the

motif F(X1)ANR(X2)(X3)I (SEQ ID NO: 60), wherein X1, X2, and X3 are any amino acid. In a further embodiment, X1 is a hydrophobic amino acid, including but not limited to V, A, or I. In one embodiment, at least one of X2 and X3 is a polar amino acid, including but not limited to L or K. In another embodiment, the EB domain comprises the amino acid sequence at least 50%, 55%, 60%, 65%, 70%, 75%, 80%, 85%, 90%, 91%, 92%, 93%, 94%, 95%, 96%, 97%, 98%, or 100% identical along to the amino acid sequence of any one of SEQ ID NOS:1-9, 11, and 13. In a further embodiment, the EB domain comprises the amino acid sequence at least 50%, 55%, 60%, 65%, 70%, 75%, 80%, 85%, 90%, 91%, 92%, 93%, 94%, 95%, 96%, 97%, 98%, or 100% identical along the length of SEQ ID NO:13, wherein the highlighted residues are not modified.

In one embodiment, the amino acid linker comprises a sequence that may include, but is not limited to, a sequence having the amino acid sequence selected from the group consisting of SEQ ID NO:14-33. In a further embodiment, the amino acid linker comprises the amino acid sequence of SEQ ID NO:33. In another embodiment, the Cas9 domain comprises the amino acid sequence at least 50%, 55%, 60%, 65%, 70%, 75%, 80%, 85%, 90%, 91%, 92%, 93%, 94%, 95%, 96%, 97%, 98%, or 100% identical to the amino acid sequence of any one of SEQ ID NO:34 or SEQ ID NO:40-57. In a specific embodiment, the Cas9 domain comprises the amino acid sequence at least 50%, 55%, 60%, 65%, 70%, 75%, 80%, 85%, 90%, 91%, 92%, 93%, 94%, 95%, 96%, 97%, 98%, or 100% identical to the amino acid sequence of SEQ ID NO:34.

In one embodiment, the composition further comprises a localization domain. In another embodiment, the localization domain comprises the amino acid sequence at least 50%, 55%, 60%, 65%, 70%, 75%, 80%, 85%, 90%, 91%, 92%, 93%, 94%, 95%, 96%, 97%, 98%, or 100% identical to the amino acid sequence of SEQ ID NO:35. In one embodiment, the composition further comprises a detectable domain. In a further embodiment, the detectable domain comprises the amino acid sequence at least 50%, 55%, 60%, 65%, 70%, 75%, 80%, 85%, 90%, 91%, 92%, 93%, 94%, 95%, 96%, 97%, 98%, or 100% identical to the amino acid sequence of SEQ ID NO:36.

In one embodiment, the polypeptide comprises the amino acid sequence at least 50%, 55%, 60%, 65%, 70%, 75%, 80%, 85%, 90%, 91%, 92%, 93%, 94%, 95%, 96%, 97%, 98%, or 100% identical to the amino acid sequence of SEQ ID NO:37 or SEQ ID NO:38. In another embodiment, the composition is bound to a scaffold, including but not limited to a nanoparticle, virus-like particle, or other polypeptide scaffold.

In other aspects, the disclosure provides nucleic acids encoding the polypeptide of any embodiment or combination of embodiments of the disclosure, expression vectors comprising the nucleic acids of the disclosure operatively linked to a suitable control sequence, and host cells comprising a nucleic acid or expression vector of the disclosure. In one embodiment, the host cell is capable of stably expressing the polypeptide. In a further embodiment, the host cell further comprises one or more guide RNAs (gRNA) selective for one or more particular genes, a nucleic acid encoding the one or more guide RNAs, and/or an expression vector comprising a nucleic acid encoding the one or more guide RNAs operatively linked to a suitable control sequence. In another embodiment, the host cell comprises an expression vector comprising a nucleic acid encoding the one or more guide RNAs operatively linked to a suitable control sequence, wherein the control sequence comprises a TATA box within 50-100 base pairs of the nucleic acid encoding the one or more guide RNAs.

In a further aspect, the disclosure provides pharmaceutical compositions comprising the composition, nucleic acid, expression vector, and/or host cell of any embodiment or combination of embodiments herein, and a pharmaceutically acceptable carrier. In one embodiment, the pharmaceutical composition further comprises a nucleic acid encoding the one or more guide RNAs operatively linked to a suitable control sequence, wherein the control sequence comprises a TATA box within 50-100 base pairs of the nucleic acid encoding the one or more guide RNAs.

In another aspect, the disclosure provides kits, comprising

(a) an active composition, nucleic acid, expression vector, host cell, and/or pharmaceutical composition of any embodiment or combination of embodiments disclosed herein; and

(b) a control composition, nucleic acid, expression vector, host cell, and/or pharmaceutical composition that is identical to the active composition, the active nucleic acid, the active expression vector, the active host cell, and/or the pharmaceutical composition, except that the EB domain is inactive (i.e.: does not bind to EED), and/or the control nucleic acid encodes an inactive EB domain. In one embodiment, the inactive EB domain comprises the amino acid sequence at least 50%, 55%, 60%, 65%, 70%, 75%, 80%, 85%, 90%, 91%, 92%, 93%, 94%, 95%, 96%, 97%, 98%, or 100% identical to the amino acid sequence of SEQ ID NO:13, wherein the highlighted residues are modified to polar or charged amino acids.

(SEQ ID NO: 13)
MINEIKKNAQERMDETVEQLKNELSKVRTGGGGTEERRLELAKQVVFAANR
ALIRVRTIALEAAWRLRMLGSDKEVNKRDISQALEEIEKLTKVAAKKIKEV
LEAKIKELREVMAVN.

In another embodiment, the inactive EB domain comprises the amino acid sequence at least 50%, 55%, 60%, 65%, 70%, 75%, 80%, 85%, 90%, 91%, 92%, 93%, 94%, 95%, 96%, 97%, 98%, or 100% identical to the amino acid sequence of SEQ ID NO:10, 12, or 39, wherein the highlighted residues are not modified

>EB15.2NC
(SEQ ID NO: 10)
HMGQRWELALQRFWDYLRWVQTLSEQVQEELLSDKAIEELAALAKETERE
LRNYIAELSKQLTPVAEETKRQLATTLVEVANRLKETMRTIMLELLRYRI
AVNALNGQSTEDLRRNLAENLRKSRDDLLITADKLQRVLAVYQAGALE
>EB22.2NC
(SEQ ID NO: 12)
HMINEIKKNAQERMDETVEQLKNELSKVRTGGGGTEERRLELAKQVVEAA
NRALERVRTIALEAAWRLRMLGSDKEVNKRDISQALEEIEKLIKVAAKKI
KEVLEAKIKELREVLE
(SEQ ID NO: 39)
MINEIKKNAQERMDETVEQLKNELSKVRTGGGGTEERRLELAKQVVEAAN
RALERVRTIALEAAWRLRMLGSDKEVNKRDISQALEEIEKLTKVAAKKIK
EVLEAKIKELREVMAVN

In another aspect, the disclosure provides method for using of the composition, the nucleic acid, the expression vector, the host cell, the pharmaceutical composition, and/or the kit of any embodiment or combination of embodiments disclosed herein for gene activation in a biological cell. In one embodiment, the method comprises

(a) providing the host cell of embodiment or combination of embodiments disclosed herein, which comprises the expression vector and/or the nucleic acid of embodiment or combination of embodiments disclosed herein;

(b) contacting the host cell with a guide RNA (gRNA) selective for a gene to be activated, including but not limited to adding the gRNA at the time of gene activation, or providing host cells that express the gRNA (including but not limited to host cells transfected with a viral construct or transiently or stably transfected with a plasmid, in each case having an appropriate promoter (including but not limited to u6) controlling gRNA expression); and

(c) culturing the cells under conditions suitable to promote expression of the polypeptide in the host cell, wherein the polypeptide directs PRC2 disruption at the gene targeted by the gRNA, thus activating the gene.

In another embodiment, the method comprises

(a) providing a host cell comprising a composition of any embodiment or combination of embodiments disclosed herein and one or more guide RNA (gRNA) selective for a gene(s) to be activated; and

(b) culturing the cells under conditions suitable to promote targeting of the gene(s) to be activated with the gRNA, wherein the composition directs PRC2 disruption at the gene targeted by the gRNA, thus activating the gene.

In one embodiment, the biological cell is present within a subject having glioblastoma, wherein the gene targeted by the gRNA comprises the p16 gene, and wherein the gene activation serves to treat the glioblastoma. In another embodiment, the one or more gRNA is encoded by a nucleic acid operatively linked to a suitable control sequence, wherein the control sequence comprises a TATA box within 50-100 base pairs of the nucleic acid encoding the gRNA.

### DESCRIPTION OF THE FIGURES

FIG. 1A-J. EBdCas9 targets TBX18 upregulation. A. Model of EBdCas9 precise elimination of PRC2 activity in targeted loci B. EBdCas9mCherry™ and EBNCdCas9mCherry™ construct under Tet On operator. C. Generation of stable EBdCas9 or NCdCas9 transgenic iPSC lines after 3D induction of 1 mg/ml Doxycycline (Dox). D. Immunoblot analysis of EBdCas9 and NCdCas9 whole cell lysate after 3D Dox induction. E. Integrative genomic viewer of TBX18 H3K27me3 and H3K4me3 promoter tiling. F. Timeline of EBdCas9 or NCdCas9 induction and gRNA transfection. G-I. RT-qPCR analysis of TBX18 or Oct 4 expression for EBdCas9 and NCdCas9 normalized to beta-Actin and calculated as relative fold increase compared to no guide (induced with Dox) of each respected cell line (G) after cocktail TBX18 gRNA transfection with either g1,2,7,8 or g3,4,5,6 TBX18 promoter tiling, (H) after individual TBX18 gRNA (1-8) transfection. (I) after individual transfection of TBX18 gRNA g5 and g6. *p<0.05, **p<0.01, ***p<0.001 one-way ANOVA performed. n=3 biological replicates. J. Immunofluorescent imaging of EBdCas9 WTC and NCdCas9 WTC for either no guide or after transfection with TBX18 gRNA 6(TBX18 g6). Blue-Dapi, Green-Oct4, Far red-TBX18, Scale bar values.

FIG. 2A-E. EBdCas9 causes epigenetic remodeling and maintains epigenetic memory on TBX18. A. EBdCas9 and NCdCas9 timeline for dox induction, gRNA transfection and analysis: RT-qPCR or ChIPqPCR using the antibodies mCherry™, EZH2 and H3K27me3 and analyzing TBX18 g6 DNA region ˜1.0 kb upstream of TSS (150 bp). B. RT-qPCR (left panel) of dCas9 or TBX18 relative fold increase after 3D dox induction (dCas9) or 3D TBX18 g6 RNA transfection (TBX18) normalized to beta-Actin and compared to no guide (Dox induced for TBX18) of each respected cell line. ChIPqPCR (right panel) of induced (+Dox) EBdCas9 and NCdCas9 after 3D transfection with TBX18 g6 RNA (+g6) or no transfection (−g). Normalized to input and H3 and compared to −g relative fold change. Antibodies that were used for ChIP are listed above the graphs (mCherry™, EZH2, H3K27me3) and the genomic region analyzed by qPCR includes TBX18 g6 locus. *p<0.05, **p<0.01, ***p<0.001 one-way ANOVA performed. n=3 biological replicates. C. EBdCas9 timeline for measuring epigenetic memory. EBdCas9 induction and TBX18 g6 RNA transfection exactly as in A. On Day 3 EBdCas9 media is replaced with no Dox (−EBdCas9) for 3 days and analyzed on Day 5 for RT-qPCR and ChIPqPCR as described in A. D. RT-qPCR analysis (left) of EBdCas9 and TBX18 for 3 days (3D) or 5 days (5D) while inducing with Dox (+) or not (−) and in the presence of TBX18 g6 RNA (g6) (+) or not (−). ChIPqPCR (right panel) of no guide (−g), 3 days (3D) or 5 days (5D) EBdCas9 either induced with Dox (+) or not (−) or transfected with TBX18 g6 RNA (+) or not (−). ChIP and qPCR assays were exactly as in B (+Dox). Normalized to respected input and H3. *p<0.05, **p<0.01, ***p<0.001 one-way ANOVA performed. n=3 biological replicates. E. Model of EBdCas9 epigenetic remodeling and memory.

FIG. 3A-H. EBdCas9 de-repressed PRC2 locus to reveal a far TBX18 TATAbox. A. Tiling of EBdCas9/+g6 ChIP (mCherry™) on TBX18 genomic loci(bp) relative to TSS (listed above nucleosome) using qPCR; solid red lines are 3D and dash red lines are 5D (as described in 2C-D). Each point is relative fold change TBX18 g6RNA (+g6) vs no guide (−g), normalized to respected input/H3 and compared to relative fold change using −1800 bp primer set control Normalized to respected input and H3. *p<0.05, **p<0.01, ***p<0.001 one-way ANOVA performed. n=3 biological replicates. B. Tiling of EBdCas9/+g6 ChIP; Upper panel (H3K27me3 and EZH2) on TBX18 genomic loci(bp) relative to TSS, exactly as described in A; solid black and yellow lines are 3D and das lines are 5D for H3K27me3 and EZH2 respectively. Lower panel: EED and JARID 2; 3D timepoint only, exactly as described in A. C. ChIPqPCR of EBdCas9 after 3D transfection with TBX18 g6 RNA (+g6) or no transfection (−g). Normalized to input and H3. Antibodies that used for ChIP are listed above the graphs (H3K27ac and p300) and the genomic region analyzed by qPCR is TBX18 g6 locus. *p<0.05, **p<0.01, ***p<0.001 one-way ANOVA performed. n=3 biological replicates. D. Element Navigation Tool for detection of core promoter elements: Given TBX18 promoter region reveals a possible combination of TATAbox (Blue; left) 50 bp downstream and mammalian initiator factor (Cyan; right) ˜70 bp downstream of TBX18 g6 locus. SEQ ID NO:61 is input sequence position 79 to 110; SEQ ID NO:62 is input sequence position 79 to 112; SEQ ID NO:63 is input sequence position 79 to 119. E. ChIPqPCR of EBdCas9 after 3D transfection with TBX18 g6 RNA (+g6) or no transfection (−g). Normalized to input/H3 and relative fold increase compared to no guide (−g). Antibodies that used for ChIP are listed above the graphs (Pol II CTD and Pol II Ser 5P+) and the genomic region analyzed by qPCR is TBX18 g6 locus as in C. *p<0.05, **p<0.01, ***p<0.001 one-way ANOVA performed. n=3 biological replicates. F. Tiling of EBdCas9/+g6 ChIP (Pol II CTD) on TBX18 genomic loci(bp) relative to TSS (listed above nucleosome) using qPCR exactly as in A; solid green lines are 3D and dash green lines are 5D (as described in 2C-D). G. RNA expression of TBX18 TATA box region using RT-qPCR after 3D transfection with TBX18 g6 RNA (plus g) or no transfection (no g). primer sets used as in ChIPqPCR. *normalized to −1800 bp DNA region and compared to no guide. H. EBdCas9 model for revealing far TATAbox region.

FIG. 4A-K. EBdCas9 targets CDKN2A (p16) upregulation. A. Integrative genomic viewer of CDKN2A H3K27me3 and H3K4me3 promoter tiling. B. Timeline of EBdCas9 or NCdCas9 induction and gRNA transfection. C. RT-qPCR analysis of EBdCas9 after 3D transfection with single gRNA (g1-g8) normalized to beta actin and relative fold increase compared to no guide (g1). D. Immunofluorescent imaging of EBdCas9 after 3D transfection with single p16 gRNA 1 (+g1) or no transfection (−g). Blue-Dapi, Green-Oct4, Far red-TBX18, Scale bar values. E. Cell count of EBdCas9 (left panel) after 3D p16 g1 transfection (+g1) compared to no guide (−g) 35 mm plate was divided into 4 quadrants, each quadrant was counted 3 times and average was taken. Scale bars: 100 um. area count/scale bar. WTC EBdCas9 cell morphology (right panel) 3D post transfection with p16g1 (+g1) compared to no guide (−g) Scale bar values. F. Growth curve of EBdCas9 transfected with p16 g1 compared to no guide. Times points are every 6h measured by Alamar Blue fluorecin. G. RT-qPCR (left panel) of dCas9 or p16 relative fold increase of EBdCas9 and NCdCas9 after 3D dox induction and p16 g1 transfection. samples were normalized to beta-Actin and compared to no guide of each respected cell line. ChIPqPCR (right panel) of induced (+Dox) EBdCas9 and NCdCas9 after 3D transfection with p16 g1 RNA (+g1) or no transfection (−g). Normalized to input/H3 and compared to −g relative fold change. Antibodies that were used for ChIP are listed above the graphs (mCherry™, EZH2, H3K27me3) and the genomic region analyzed by qPCR includes p16 g1 locus. *p<0.05, ** p<0.01, ***p<0.001 one-way ANOVA performed. n=3 biological replicates. H. EBdCas9 timeline for measuring epigenetic memory as in 2C. I. RT-qPCR analysis (left) of dCas9 and p16 after 3 days (3D) or 5 days (5D) while inducing with Dox (+) or not (−) and in the presence of p16 g1 RNA (g1) (+) or not (−). ChIPqPCR (right panel) of no guide (−g), 3 days (3D) or 5 days (5D) EBdCas9 either induced with Dox (+) or not (−) or transfected with p16 g1 RNA (+) or not (−). Analysis as in G. samples were normalized to respected input/H3. *p<0.05, **p<0.01, ***p<0.001 one-way ANOVA performed. n=3 biological replicates. J. H3K4me3 tracks from a 100 kb CDKN2A represented region comparing EBdCas9−g to p16 g1 transfected cells. Normalized to input/H3. K. Tiling of EBdCas9/+g6 ChIP (mCherry™) on p16 genomic loci(bp) relative to TSS using qPCR; Each point is relative fold change p16 g1 RNA (+g1) vs no guide (−g), normalized to respected input/H3 *p<0.05, **p<0.01, ***p<0.001 one-way ANOVA performed. n=3 biological replicates.

FIG. 5A-K. Trophoblast trans-differentiation using EBdCas9. A. EPS differentiation protocol to trophoblast. B. WTC EB-Flag or WTC NC-Flag (Moody et al) were reprogramed to EPS for 2 weeks (3 passages). Bright Field colony morphology of EPS EB-Flag or NC-Flag induced with Dox on Mef for 4D (top panel) or plated on Matrigel™ in TX media for 4 days (4D) under the induction of Dox (scale/magnification). C. EPS EB-Flag were grown on Matrigel™ in TX media with (+) or without (−) dox for 4 days and analyzed by RT-qPCR. D. Immunofluorescence of EPS EB-Flag or EPS NC-Flag on MEF/LCDM media or differentiation using Matrigel™/TX media with (+) or without Dox (−). Dapi-Blue, WGA-Red, Oct4-Green, Gata3-Far red. Scale bars: 43 um. E. PCA analysis of EPS samples compared to monkey single cell RNA-seq⁵⁶. EB-Flag EPS cells were differentiating in TX media with or without dox for 4 days or 6 days or passage 3 times as extravillous cytotrophoblast (EVT). Cell types in the monkey single cell data include: Post-paTE, post-implantation parietal trophectoderm; PreL-TE, pre-implantation late TE; PreE-TE, pre-implantation early TE; ICM, inner cell mass; Pre-EPI, pre-implantation epiblast; PostE-EPI, post-implantation early epiblast; PostL-EPI, post-implantation late epiblast. F. model of EBdCas9 transdifferentiation to trophoblasts using EBdCas9 and CDX2 and GATA3 gRNA. G. Timeline of EBdCas9 or NCdCas9 induction and gRNA transfection. H. Tiling of CDX3 and GATA3 promoter and gene body gRNA. I. RT-qPCR analysis of CDX2 and GATA3 Co-transfection gRNAs in the presence of EBdCas9 or NCdCas9 induction (+Dox). J.PCA analysis of EBdCas9 CDX2/GATA3 gRNA co-transfection RNAseq compared to WTC dataset⁵⁷. K. Immunofluorescence of EBdCas9 3D post CDX2g5 RNA and Gata3 g5 RNA (g5,g5) transfection (cytotrophoblast, CT) compared to no guide, and further 6 days (6D) differentiation to either extravillous cytotrophoblasts (EVT) using TGFbi and nuerogulin (NRG1) or Syncytiotrophoblast (ST) using forskolin. Dapi-blue, CGB-far red. White arrows depicts multinucleated cells. Images processed at 63× magnification.

### DETAILED DESCRIPTION

All references cited are herein incorporated by reference in their entirety. As used herein, the singular forms “a”, “an” and “the” include plural referents unless the context clearly dictates otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

In one aspect the disclosure provides compositions, comprising:

(a) an embryonic ectoderm development (EED) polypeptide binder (EB) domain; and

(b) a CRISPR associated protein 9 (CAS9) domain linked to the EB domain.

The EB domain and the CAS9 domain can be linked by any suitable means, such as by covalent binding or they may be expressed as a fusion protein. In one embodiment, the EB domain and the CAS9 domain are expressed in a fusion protein, and may be separated by an amino acid linker connecting the EB domain and the CAS domain.

As disclosed in the examples that follow, the inventors have discovered that the fusion proteins disclosed herein can be used, for example, to direct PRC2 disruption at precise loci using gRNA and by that locally reduce H3K27me3 marks to promote single gene activation. Such precise control of epigenetic regulation can be used, for example, to treat human diseases or direct cell fate determination free of traditional chemical drugs or DNA manipulation, and as a research tool will for the study of the epigenetic memory of loss of specific H3K27 methyl marks.

Any suitable the EB domains and CAS9 domains may be used in the compositions and fusion proteins of the disclosure. In one embodiment, the EB domain comprises the motif F(X1)ANR(X2)(X3)I (SEQ ID NO: 60), wherein X1, X2, and X3 are any amino acid. This domain serves as the interface with EED. In one embodiment, X1 is a hydrophobic, neutral amino acid (i.e.: Norleucine, G, M, A, V, L, I), including but not limited to V, A, or I. In another embodiment, one or both of X2 and X3 are a polar amino acid (i.e., K, R, H, G, S, T, C, Y, N, Q, D, E) including but not limited to L or K.

In another embodiment, the EB domain comprises the amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NOS:1-11, and 13.

>EB15
(SEQ ID NO: 1)
HMGQRWELALQRFWDYLRWVQTLSEQVQEELLSDKAIEELAALAKETERELRNYIAELSK
QLTPVAEETKRQLATTLVFVANRLKITMRTIMLELLWYRIAVNALNGQSTEDLRRNLAEN
LRKSRDDLLITADKLQRVLAVYQAGALE
>EB16
(SEQ ID NO: 2)
HMGQRWELALQRFWDYLRWVQTLSEQVQEELLTKQVIRELSELRSNTLRELAAYKSELEE
QLTPVAEETRARLSKELATTAKALLFVMNRILIALRTYILAVLWMDGISTEKLRVQLASD
LRQLRDKLLRAADELQKVLAVYQAGALE
>EB17
(SEQ ID NO: 3)
HMGGWRREYPPITSDQQRQEYKRNFDTGLREAARLVFILNRIRIQLRILILELIWADEES
RRYKQAADEYNRLKQVKGSADYKSKRDIVLELAKKLEHIAKMVKDYDRQKTLE
>EB18
(SEQ ID NO: 4)
HMIREALKDAQEKMKKAVQVAEDDLSTIRTGGGGIQERRKELVDQAIHKGKEAEQSVKKI
MEEAQKELRRIRKEGEAGEDEVGKASAMLIFITNRYKITIRTLVLEKMWRLLAVLE
>EB19
(SEQ ID NO: 5)
HMGGWRREYPPITSDQQRQRYVEDSKRGAFIYNRLRIVLRTIELELIWLDIILRSLREES
EDYMRAAERYNRLKQVKGSAEYKSAKNHAEQLKKKLDHLHKMVEDYLRQKTLE
>EB20
(SEQ ID NO: 6)
HMTSKQRQVFIANRRKISARTAILELMWQDSERNRRLAQREVNKAPQESKEKLQKILDQL
VADKDAEKLE
>EB21
(SEQ ID NO: 7)
HMSMQEEDTFRELRIFLRQVIHRLAIREALRVFTKPVDPDEVPDYVIVIEQPMDLSSVIS
KIDLHKYLIVKDYLRDIDLIMRNALKYNPRASFKNNRIAIAARTLALEAYWIIEMELDRK
FEQLAEEIQKSRLE
>EB22
(SEQ ID NO: 8)
HMINEIKKNAQERMDETVEQLKNELSKVRIGGGGTEERRLELAKQVVFAANRALIRVRTI
ALEAAWRLLMLGSDKEVNKRDISQALEEIEKLIKVAAKKIKEVLEAKIKELREVLE
>EB15.2
(SEQ ID NO: 9)
HMGQRWELALQRFWDYLRWVQTLSEQVQEELLSDKAIEELAALAKETERELRNYIAELSK
QLTPVAEETKRQLATTLVFVANRLKITMRTIMLELLRYRIAVNALNGQSTEDLRRNLAEN
LRKSRDDLLITADKLQRVLAVYQAGALE
>EB22.2 bb
(SEQ ID NO: 11)
HMINEIKKNAQERMDETVEQLKNELSKVRIGGGGTEERRLELAKQVVFAANRALIRVRTI
ALEAAWRLRMLGSDKEVNKRDISQALEEIEKLIKVAAKKIKEVLEAKIKELREVLE
EB
(SEQ ID NO: 13)
MINEIKKNAQERMDETVEQLKNELSKVRIGGGGTEERRLELAKQVVFAANRALIRVRTIALEAAWRLR
MLGSDKEVNKRDISQALEEIEKLTKVAAKKIKEVLEAKIKELREVMAVN

In another embodiment, the EB domain comprises the amino acid sequence at least 50%, 55%, 60%, 65%, 70%, 75%, 80%, 85%, 90%, 91%, 92%, 93%, 94%, 95%, 96%, 97%, 98%, or 100% identical to the amino acid sequence of SEQ ID NO:13, wherein the highlighted residues are not modified.

In one embodiment, amino acid substitutions relative to the EB domain or any other reference peptide domains described herein are conservative amino acid substitutions. As used herein, “conservative amino acid substitution” means a given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are known. Polypeptides comprising conservative amino acid substitutions can be tested in any one of the assays described herein to confirm that a desired activity, e.g. antigen-binding activity and specificity of a native or reference polypeptide is retained. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into H is; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

Any suitable amino acid linker may be used in the fusion polypeptides of the disclosure. In one non-limiting embodiment, the linkers vary from 2 to 31 amino acids of any primary sequence in length and do not impose any constraints on the conformation or interactions of the linked partners. In various further embodiments, the linkers vary from 2-30, 2-29, 2-28, 2-27, 2-26, 2-25, 2-24, 2-23, 2-22, 2-21, 2-20, 2-19, 2-18, 2-17, 2-16, 2-15, 2-14, 2-13, 2-12, 2-11, 2-10, 2-9, 2-8, 2-7, 2-6, 2-5, 2-4, 2-3, 3-31, 3-30, 3-29, 3-28, 3-27, 3-26, 3-25, 3-24, 3-23, 3-22, 3-21, 3-20, 3-19, 3-18, 3-17, 3-16, 3-15, 3-14, 3-13, 3-12, 3-11, 3-10, 3-9, 3-8, 3-7, 3-6, 3-5, 3-4, 4-31, 4-30, 4-29, 4-28, 4-27, 4-26, 4-25, 4-24, 4-23, 4-22, 4-21, 4-20, 4-19, 4-18, 4-17, 4-16, 4-15, 4-14, 4-13, 4-12, 4-11, 4-10, 4-9, 4-8, 4-7, 4-6, 4-5, 5-31, 5-30, 5-29, 5-28, 5-27, 5-26, 5-25, 5-24, 5-23, 5-22, 5-21, 5-20, 5-19, 5-18, 5-17, 5-16, 5-15, 5-14, 5-13, 5-12, 5-11, 5-10, 5-9, 5-8, 5-7, 5-6, 6-31, 6-30, 6-29, 6-28, 6-27, 6-26, 6-25, 6-24, 6-23, 6-22, 6-21, 6-20, 6-19, 6-18, 6-17, 6-16, 6-15, 6-14, 6-13, 6-12, 6-11, 6-10, 6-9, 6-8, 6-7, 7-31, 7-30, 7-29, 7-28, 7-27, 7-26, 7-25, 7-24, 7-23, 7-22, 7-21, 7-20, 7-19, 7-18, 7-17, 7-16, 7-15, 7-14, 7-13, 7-12, 7-11, 7-10, 7-9, 7-8, 8-31, 8-30, 8-29, 8-28, 8-27, 8-26, 8-25, 8-24, 8-23, 8-22, 8-21, 8-20, 8-19, 8-18, 8-17, 8-16, 8-15, 8-14, 8-13, 8-12, 8-11, 8-10, 8-9, 9-31, 9-30, 9-29, 9-28, 9-27, 9-26, 9-25, 9-24, 9-23, 9-22, 9-21, 9-20, 9-19, 9-18, 9-17, 9-16, 9-15, 9-14, 9-13, 9-12, 9-11, 9-10, 10-31, 10-30, 10-29, 10-28, 10-27, 10-26, 10-25, 10-24, 10-23, 10-22, 10-21, 10-20, 10-19, 10-18, 10-17, 10-16, 10-15, 10-14, 10-13, 10-12, 10-11, 11-31, 11-30, 11-29, 11-28, 11-27, 11-26, 11-25, 11-24, 11-23, 11-22, 11-21, 11-20, 11-19, 11-18, 11-17, 11-16, 11-15, 11-14, 11-13, 11-12, 12-31, 12-30, 12-29, 12-28, 12-27, 12-26, 12-25, 12-24, 12-23, 12-22, 12-21, 12-20, 12-19, 12-18, 12-17, 12-16, 12-15, 12-14, 12-13, 13-31, 13-30, 13-29, 13-28, 13-27, 13-26, 13-25, 13-24, 13-23, 13-22, 13-21, 13-20, 13-19, 13-18, 13-17, 13-16, 13-15, 13-14, 14-31, 14-30, 14-29, 14-28, 14-27, 14-26, 14-25, 14-24, 14-23, 14-22, 14-21, 14-20, 14-19, 14-18, 14-17, 14-16, 14-15, 15-31, 15-30, 15-29, 15-28, 15-27, 15-26, 15-25, 15-24, 15-23, 15-22, 15-21, 15-20, 15-19, 15-18, 15-17, 15-16, 16-31, 16-30, 16-29, 16-28, 16-27, 16-26, 16-25, 16-24, 16-23, 16-22, 16-21, 16-20, 16-19, 16-18, 16-17, 17-31, 17-30, 17-29, 17-28, 17-27, 17-26, 17-25, 17-24, 17-23, 17-22, 17-21, 17-20, 17-19, 17-18, 18-31, 18-30, 18-29, 18-28, 18-27, 18-26, 18-25, 18-24, 18-23, 18-22, 18-21, 18-20, 18-19, 19-31, 19-30, 19-29, 19-28, 19-27, 19-26, 19-25, 19-24, 19-23, 19-22, 19-21, 19-20, 20-31, 20-30, 20-29, 20-28, 20-27, 20-26, 20-25, 20-24, 20-23, 20-22, 20-21, 21-31, 21-30, 21-29, 21-28, 21-27, 21-26, 21-25, 21-24, 21-23, 21-22, 22-31, 22-30, 22-29, 22-28, 22-27, 22-26, 22-25, 22-24, 22-23, 23-31, 23-30, 23-29, 23-28, 23-27, 23-26, 23-25, 23-24, 24-31, 24-30, 24-29, 24-28, 24-27, 24-26, 24-25, 25-31, 25-30, 25-29, 25-28, 25-27, 25-26, 26-31, 26-30, 26-29, 26-28, 26-27, 27-31, 27-30, 27-29, 27-28, 28-31, 28-30, 28-29, 29-31, 29-30, or 30-31 amino acids of any primary sequence in length. The linkers can be designed as appropriate for an intended use. By way of various non-limiting examples:
Gly-rich linkers are flexible, connecting various domains in a single protein without interfering with the function of each domain.
Gly-rich linkers may be employed to form stable covalently linked dimers, and to connect two independent domains that create a ligand-binding site or recognition sequence.
Serine allows a coiled structure, but can be swapped with Gln, Arg, Glu, Ser, and Pro amino acids.
rigid spacers include Pro, Arg, Phe, Thr, Glu, and Gln residues.
Thr, Ser, Gly, and Ala are preferred residues in some linker embodiments.
Flexible Gly-rich regions can generate loops that connect domains.

In one non-limiting embodiment, the amino acid linker comprises a sequence that may include, but is not limited to, a sequence selected from the group consisting of SEQ ID NO:14-33.

(SEQ ID NO: 14)
(SGGGG)₁₋₆
(SEQ ID NO: 15)
(GGS)₁₋₅
(SEQ ID NO: 16)
GGGGSLVPRGSGGGGS
(SEQ ID NO: 17)
GSGSGS
(SEQ ID NO: 18)
(GS)₈
(SEQ ID NO: 19)
GGSGGHMGSGG
(SEQ ID NO: 20)
GGSGGSGGSGG
(SEQ ID NO: 21)
GGSGG
(SEQ ID NO: 22)
GGSGGGGG
(SEQ ID NO: 23)
GSGSGSGS
(SEQ ID NO: 24)
GSGSGSGSGSGSGSGSGSGSGSGSGSGSGSG
31 amino acids glycine-serine rich linker
(SEQ ID NO: 25)
GGGSEGGGSEGGGSEGGG
(SEQ ID NO: 26)
AAGAATAA
(SEQ ID NO: 27)
GGGGG
(SEQ ID NO: 28)
GGSSG
(SEQ ID NO: 29)
GSGGGTGGGSG
(SEQ ID NO: 30)
GT
(SEQ ID NO: 31)
GSGSGSGSGGSG
(SEQ ID NO: 32)
GSGGSGSGGSGGSG
(SEQ ID NO: 33)
SGGGGSRGGGSGGGG[SGGGG][SGGGG][SGGGG]
(bracketed residues are optional)

In one non-limiting embodiment, the amino acid linker comprises the amino acid sequence of SEQ ID NO:33. In one such embodiment, the optional residues are all present. In other embodiments, the optional residues are absent in whole or in part.

Any suitable Cas9 protein or active fragment thereof can be used as the Cas9 domain in the compositions or fusion proteins of the disclosure. Many Cas9 proteins or active fragments thereof are known that have a nuclease activity to generate a double stranded break in a genomic target of interest when in the presence of an appropriate guide RNA(s) (gRNA). In various non-limiting embodiments, the Cas9 domain comprises the amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of any one of SEQ ID NO:34 or SEQ ID NO: 40-57.

SEQ ID NO: 34
MDKKYSIGLAIGINSVGWAVITDEYKVPSKKFKVLGNTDRHSIKKNLIGALLFDSGETAEATRLKRTA
RRRYTRRKNRICYLQEIFSNEMAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSNFDLAEDAKLQLSKDTYD
DDLDNLLAQIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEETITPW
NFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLSRKLINGIRDKQSGKTIL
DFLKSDGFANRNFMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYL
QNGRDMYVDQELDINRLSDYDVDAIVPQSFLKDDSIDNKVLTRSDKNRGKSDNVPSEEVVKKMKNYWR
QLLNAKLITQRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGIALIKKYPKLESEFVYGDYKVYDVRK
MIAKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYISTKEVLDATLIHQSITGLYETRI
DLSQLGGD.
uniprot accession number
CRISPR-Cas9
Q03JI6
Streptococcus thermophiles Cas9
(SEQ ID NO: 40)
MTKPYSIGLDTGINSVGWAVTTDNYKVPSKKMKVLGNTSKKYIKKNLLGVLLFDSGITAE
GRRLKRTARRRYTRRRNRILYLQEIFSTEMATLDDAFFQRLDDSFLVPDDKRDSKYPIFG
NLVEEKAYHDEFPTIYHLRKYLADSTKKADLRLVYLALAHMIKYRGHFLIEGEFNSKNND
IQKNFQDFLDTYNAIFESDLSLENSKQLEEIVKDKISKLEKKDRILKLFPGEKNSGIFSE
FLKLIVGNQADFRKCFNLDEKASLHFSKESYDEDLETLLGYIGDDYSDVFLKAKKLYDAI
LLSGFLTVTDNETEAPLSSAMIKRYNEHKEDLALLKEYIRNISLKTYNEVEKDDTKNGYA
GYIDGKTNQEDFYVYLKKLLAEFEGADYFLEKIDREDFLRKQRTFDNGSIPYQIHLQEMR
AILDKQAKFYPFLAKNKERIEKILTFRIPYYVGPLARGNSDFAWSIRKRNEKITPWNFED
VIDKESSAEAFINRMTSFDLYLPEEKVLPKHSLLYETFNVYNELTKVRFIAESMRDYQFL
DSKQKKDIVRLYFKDKRKVTDKDIIEYLHAIYGYDGIELKGIEKQFNSSLSTYHDLLNII
NDKEFLDDSSNEAIIEEIIHTLTIFEDREMIKQRLSKFENIFDKSVLKKLSRRHYTGWGK
LSAKLINGIRDEKSGNTILDYLIDDGISNRNFMQLIHDDALSFKKKIQKAQIIGDEDKGN
IKEVVKSLPGSPAIKKGILQSIKIVDELVKVMGGRKPESIVVEMARENQYINQGKSNSQQ
RLKRLEKSLKELGSKILKENIPAKLSKIDNNALQNDRLYLYYLQNGKDMYTGDDLDIDRL
SNYDIDHIIPQAFLKDNSIDNKVLVSSASNRGKSDDVPSLEVVKKRKTFWYQLLKSKLIS
QRKFDNLTKAERGGLSPEDKAGFIQRQLVETRQITKHVARLLDEKFNNKKDENNRAVRTV
KIITLKSTLVSQFRKDFELYKVREINDFHHAHDAYLNAVVASALLKKYPKLEPEFVYGDY
PKYNSFRERKSATEKVYFYSNIMNIFKKSISLADGRVIERPLIEVNEETGESVWNKESDL
ATVRRVLSYPQVNVVKKVEEQNHGLDRGKPKGLFNANLSSKPKPNSNENLVGAKEYLDPK
KYGGYAGISNSFTVLVKGTIEKGAKKKITNVLEFQGISILDRINYRKDKLNFLLEKGYKD
IELIIELPKYSLFELSDGSRRMLASILSTNNKRGEIHKGNQIFLSQKFVKLLYHAKRISN
TINENHRKYVENHKKEFEELFYYILEFNENYVGAKKNGKLLNSAFQSWQNHSIDELCSSF
IGPTGSERKGLFELTSRGSAADFEFLGVKIPRYRDYTPSSLLKDATLIHQSVTGLYETRI
DLAKLGEG
A0A1L8RGR8
Enterococcus canis
(SEQ ID NO: 41)
MKQNKELVNIGFDIGIASVGWSVVSKQSGKILETGVSIFFSGTASKNEERRSFRQARRLL
RRRKNRISDLKILLEENGFRIAKLNQLVTPYELRVRGLNEQLSKEELSVALLHLVKRRGI
SYSLEDSEGEGDNQTSYKQSVSINQKLLKEKTPGEIQLERLEKYGKIRGQVKDLQEENAA
VLMNVFPNTAYVREAELILLKQKEYYSEITDNFIKEATALISRKREYFVGPGSEKSRTDY
GIYRTDGTKLDNLFEILIGKDKIFPNEFRAAGNSYTAQLYNLLNDLNNLKIKTLEDGKLT
KDQKLSIIEELKTTTKKVNMMQLIKKIAKAEESDISGYRIDRNDKPEIHSMAIFYKVRKK
FLEQEIDINDWPIDFLDILGRVLTLNTENGEIRRSLTELKKDYIFLDETLIELIINSKDS
FKLTSNQKWHRFSLKTMQLLIPELLNSSKEQMTILTELGLLHENKQDYSNKTKIDVKNLT
ENIYNPVVRKSVKQAMDIFNSLFKKYPNIAYLVVEMPRDEAEDEVEQKKQAQKFQKENEA
EKEKSLKEFQELAGVSDSQLENQIYKRRKLRMKIRLWYQQLGKCPYSGKTIAAEDLFWFD
HLFEIDHVIPLSISYDDGQNNKVLCYSEMNQEKGQKTPYGFMQSGKGQGFSALQAMLKSN
SRMSGAKKRNLLFTEDINDIEVRKRFIARNLVDTRYASRIVLNELQQFTRSKQLDTKVTV
IRGKFTSKLRETWRLNKSRETHHHHAVDATIIAVSPMLKLWERNAEIIPMKVNENVVDIK
TGEILTDKVYQEEMYQLPYASLLEDIAVMENKIKFHHQVDKKMNRKVSDATIYATRSAKV
GKDKEPQNYVLGKIKDIYDTKEYENFKKIYDKDKSKFLMQQLDPMTFEKLEKVLKEYPDF
EEVQQDNGRVKRIPISPFELYRREKGPITKFAKRNNGPAIKSVKYYDSKMGSAIDITPQT
AKNKKVVLQSLKPWRTDVYFNQETKEYEIMGIKYSDMQYLNGNYGITNERYKEIQREEGV
ADNSEFMMSLYRGDRIKVIDTNSDESVELLFGSRTIPTKKGYVELKPIEKTKFDSKEIVS
FYGQVTPNGQFVKKFTRNGYRLLKVNTNILGNPYYISKEGINPRNILDTGFKG
J7RUA5
Staphylococcus aureus Cas9
(SEQ ID NO: 42)
MKRNYILGLDIGITSVGYGIIDYETRDVIDAGVRLFKEANVENNEGRRSKRGARRLKRRR
RHRIQRVKKLLFDYNLLTDHSELSGINPYEARVKGLSQKLSEEEFSAALLHLAKRRGVHN
VNEVEEDTGNELSTKEQISRNSKALEEKYVAELQLERLKKDGEVRGSINRFKTSDYVKEA
KQLLKVQKAYHQLDQSFIDTYIDLLETRRTYYEGPGEGSPFGWKDIKEWYEMLMGHCTYF
PEELRSVKYAYNADLYNALNDLNNLVITRDENEKLEYYEKFQIIENVFKQKKKPTLKQIA
KEILVNEEDIKGYRVTSTGKPEFTNLKVYHDIKDITARKEIIENAELLDQIAKILTIYQS
SEDIQEELTNLNSELTQEEIEQISNLKGYTGTHNLSLKAINLILDELWHTNDNQIAIFNR
LKLVPKKVDLSQQKEIPTTLVDDFILSPVVKRSFIQSIKVINAIIKKYGLPNDIIIELAR
EKNSKDAQKMINEMQKRNRQTNERIEEIIRTTGKENAKYLIEKIKLHDMQEGKCLYSLEA
IPLEDLLNNPFNYEVDHIIPRSVSFDNSFNNKVLVKQEENSKKGNRTPFQYLSSSDSKIS
YETFKKHILNLAKGKGRISKTKKEYLLEERDINRFSVQKDFINRNLVDTRYATRGLMNLL
RSYFRVNNLDVKVKSINGGFTSFLRRKWKFKKERNKGYKHHAEDALIIANADFIFKEWKK
LDKAKKVMENQMFEEKQAESMPEIETEQEYKEIFITPHQIKHIKDFKDYKYSHRVDKKPN
RELINDTLYSTRKDDKGNTLIVNNLNGLYDKDNDKLKKLINKSPEKLLMYHHDPQTYQKL
KLIMEQYGDEKNPLYKYYEEIGNYLTKYSKKDNGPVIKKIKYYGNKLNAHLDITDDYPNS
RNKVVKLSLKPYRFDVYLDNGVYKFVTVKNLDVIKKENYYEVNSKCYEEAKKLKKISNQA
EFIASFYNNDLIKINGELYRVIGVNNDLLNRIEVNMIDITYREYLENMNDKRPPRIIKTI
ASKTQSIKKYSTDILGNLYEVKSKKHPQIIKKG
G3ECR1
Streptococcus thermophilus1Cas9
(SEQ ID NO: 43)
MLFNKCIIISINLDFSNKEKCMTKPYSIGLDIGTNSVGWAVITDNYKVPSKKMKVLGNTS
KKYIKKNLLGVLLFDSGITAEGRRLKRTARRRYTRRRNRILYLQEIFSTEMATLDDAFFQ
RLDDSFLVPDDKRDSKYPIFGNLVEEKVYHDEFPTIYHLRKYLADSTKKADLRLVYLALA
HMIKYRGHFLIEGEFNSKNNDIQKNFQDFLDTYNAIFESDLSLENSKQLEEIVKDKISKL
EKKDRILKLFPGEKNSGIFSEFLKLIVGNQADFRKCFNLDEKASLHFSKESYDEDLETLL
GYIGDDYSDVFLKAKKLYDAILLSGFLTVTDNETEAPLSSAMIKRYNEHKEDLALLKEYI
RNISLKTYNEVFKDDTKNGYAGYIDGKTNQEDFYVYLKNLLAEFEGADYFLEKIDREDFL
RKQRTFDNGSIPYQIHLQEMRAILDKQAKFYPFLAKNKERIEKILTFRIPYYVGPLARGN
SDFAWSIRKRNEKITPWNFEDVIDKESSAEAFINRMTSFDLYLPEEKVLPKHSLLYETFN
VYNELTKVRFIAESMRDYQFLDSKQKKDIVRLYFKDKRKVTDKDIIEYLHAIYGYDGIEL
KGIEKQFNSSLSTYHDLLNIINDKEFLDDSSNEAIIEEIIHTLTIFEDREMIKQRLSKFE
NIFDKSVLKKLSRRHYTGWGKLSAKLINGIRDEKSGNTILDYLIDDGISNRNFMQLIHDD
ALSFKKKIQKAQIIGDEDKGNIKEVVKSLPGSPAIKKGILQSIKIVDELVKVMGGRKPES
IVVEMARENQYTNQGKSNSQQRLKRLEKSLKELGSKILKENIPAKLSKIDNNALQNDRLY
LYYLQNGKDMYTGDDLDIDRLSNYDIDHIIPQAFLKDNSIDNKVLVSSASNRGKSDDFPS
LEVVKKRKTFWYQLLKSKLISQRKEDNLTKAERGGLLPEDKAGFIQRQLVETRQITKHVA
RLLDEKFNNKKDENNRAVRTVKIITLKSTLVSQFRKDFELYKVREINDFHHAHDAYLNAV
IASALLKKYPKLEPEFVYGDYPKYNSFRERKSATEKVYFYSNIMNIFKKSISLADGRVIE
RPLIEVNEETGESVWNKESDLATVRRVLSYPQVNVVKKVEEQNHGLDRGKPKGLFNANLS
SKPKPNSNENLVGAKEYLDPKKYGGYAGISNSFAVLVKGTIEKGAKKKITNVLEFQGISI
LDRINYRKDKLNFLLEKGYKDIELIIELPKYSLFELSDGSRRMLASILSTNNKRGEIHKG
NQIFLSQKFVKLLYHAKRISNTINENHRKYVENHKKEFEELFYYILEFNENYVGAKKNGK
LLNSAFQSWQNHSIDELCSSFIGPTGSERKGLFELTSRGSAADFEFLGVKIPRYRDYTPS
SLLKDATLIHQSVTGLYETRIDLAKLGEG
Q99ZW2
Streptococcus pyogenes Cas9
(SEQ ID NO: 44)
MDKKYSIGLDIGTNSVGWAVITDEYKVPSKKFKVLGNTDRHSIKKNLIGALLFDSGETAE
ATRLKRTARRRYTRRKNRICYLQEIFSNEMAKVDDSFFHRLEESFLVEEDKKHERHPIFG
NIVDEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLFGN
LIALSLGLIPNFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELH
AILRRQEDFYPFLKDNREKIEKILTFRIPYYVGPLARGNSRFAWMTRKSEETITPWNFEE
VVDKGASAQSFIERMTNFDKNLPNEKVLPKHSLLYEYFTVYNELTKVKYVTEGMRKPAFL
SGEQKKAIVDLLFKTNRKVTVKQLKEDYFKKIECFDSVEISGVEDRFNASLGTYHDLLKI
IKDKDFLDNEENEDILEDIVLILTLFEDREMIEERLKTYAHLFDDKVMKQLKRRRYTGWG
RLSRKLINGIRDKQSGKTILDFLKSDGFANRNFMQLIHDDSLIFKEDIQKAQVSGQGDSL
HEHIANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYDVDH
IVPQSFLKDDSIDNKVLIRSDKNRGKSDNVPSEEVVKKMKNYWRQLLNAKLITQRKEDNL
TKAERGGLSELDKAGFIKRQLVETRQITKHVAQILDSRMNTKYDENDKLIREVKVITLKS
KLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDF
ATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPK
YSLFELENGRKRMLASAGELQKGNELALPSKYVNFLYLASHYEKLKGSPEDNEQKQLFVE
QHKHYLDEIIEQISEFSKRVILADANLDKVLSAYNKHRDKPIREQAENIIHLFTLINLGA
PAAFKYFDTTIDRKRYTSTKEVLDATLIHQSITGLYETRIDLSQLGGD
C9X1G5
Neisseria meningitidis Cas9
(SEQ ID NO: 45)
MAAFKPNSINYILGLDIGIASVGWAMVEIDEEENPIRLIDLGVRVFERAEVPKTGDSLAM
ARRLARSVRRLTRRRAHRLLRTRRLLKREGVLQAANFDENGLIKSLPNTPWQLRAAALDR
KLTPLEWSAVLLHLIKHRGYLSQRKNEGETADKELGALLKGVAGNAHALQTGDFRTPAEL
ALNKFEKESGHIRNQRSDYSHTFSRKDLQAELILLFEKQKEFGNPHVSGGLKEGIETLLM
TQRPALSGDAVQKMLGHCTFEPAEPKAAKNTYTAERFIWLTKLNNLRILEQGSERPLTDT
ERATLMDEPYRKSKLTYAQARKLLGLEDTAFFKGLRYGKDNAEASTLMEMKAYHAISRAL
EKEGLKDKKSPLNLSPELQDEIGTAFSLFKTDEDITGRLKDRIQPEILEALLKHISFDKF
VQISLKALRRIVPLMEQGKRYDEACAEIYGDHYGKKNTEEKIYLPPIPADEIRNPVVLRA
LSQARKVINGVVRRYGSPARIHIETAREVGKSFKDRKEIEKRQEENRKDREKAAAKFREY
FPNFVGEPKSKDILKLRLYEQQHGKCLYSGKEINLGRLNEKGYVEIDHALPFSRTWDDSF
NNKVLVLGSENQNKGNQTPYEYFNGKDNSREWQEFKARVETSRFPRSKKQRILLQKFDED
GFKERNLNDTRYVNRFLCQFVADRMRLTGKGKKRVFASNGQITNLLRGFWGLRKVRAEND
RHHALDAVVVACSTVAMQQKITRFVRYKEMNAFDGKTIDKETGEVLHQKTHFPQPWEFFA
QEVMIRVFGKPDGKPEFEEADTLEKLRTLLAEKLSSRPEAVHEYVTPLFVSRAPNRKMSG
QGHMETVKSAKRLDEGVSVLRVPLTQLKLKDLEKMVNREREPKLYEALKARLEAHKDDPA
KAFAEPFYKYDKAGNRTQQVKAVRVEQVQKTGVWVRNHNGIADNATMVRVDVFEKGDKYY
LVPIYSWQVAKGILPDRAVVQGKDEEDWQLIDDSFNFKFSLHPNDLVEVITKKARMFGYF
ASCHRGTGNINIRIHDLDHKIGKNGILEGIGVKTALSFQKYQIDELGKEIRPCRLKKRPP
VR
A0Q5Y3
Francisella novicida Cas9
(SEQ ID NO: 46)
MNFKILPIAIDLGVKNTGVFSAFYQKGTSLERLDNKNGKVYELSKDSYTLLMNNRTARRH
QRRGIDRKQLVKRLFKLIWTEQLNLEWDKDTQQAISFLFNRRGFSFITDGYSPEYLNIVP
EQVKAILMDIFDDYNGEDDLDSYLKLATEQESKISEIYNKLMQKILEFKLMKLCTDIKDD
KVSTKTLKEITSYEFELLADYLANYSESLKTQKFSYTDKQGNLKELSYYHHDKYNIQEFL
KRHATINDRILDTLLTDDLDIWNFNFEKFDFDKNEEKLQNQEDKDHIQAHLHHFVFAVNK
IKSEMASGGRHRSQYFQEITNVLDENNHQEGYLKNFCENLHNKKYSNLSVKNLVNLIGNL
SNLELKPLRKYFNDKIHAKADHWDEQKFTETYCHWILGEWRVGVKDQDKKDGAKYSYKDL
CNELKQKVTKAGLVDFLLELDPCRTIPPYLDNNNRKPPKCQSLILNPKFLDNQYPNWQQY
LQELKKLQSIQNYLDSFETDLKVLKSSKDQPYFVEYKSSNQQIASGQRDYKDLDARILQF
IFDRVKASDELLLNEIYFQAKKLKQKASSELEKLESSKKLDEVIANSQLSQILKSQHTNG
IFEQGTFLHLVCKYYKQRQRARDSRLYIMPEYRYDKKLHKYNNTGRFDDDNQLLTYCNHK
PRQKRYQLLNDLAGVLQVSPNFLKDKIGSDDDLFISKWLVEHIRGFKKACEDSLKIQKDN
RGLLNHKINIARNTKGKCEKEIFNLICKIEGSEDKKGNYKHGLAYELGVLLFGEPNEASK
PEFDRKIKKFNSIYSFAQIQQIAFAERKGNANTCAVCSADNAHRMQQIKITEPVEDNKDK
IILSAKAQRLPAIPTRIVDGAVKKMATILAKNIVDDNWQNIKQVLSAKHQLHIPIITESN
AFEFEPALADVKGKSLKDRRKKALERISPENIFKDKNNRIKEFAKGISAYSGANLTDGDF
DGAKEELDHIIPRSHKKYGTLNDEANLICVTRGDNKNKGNRIFCLRDLADNYKLKQFETT
DDLEIEKKIADTIWDANKKDFKFGNYRSFINLTPQEQKAFRHALFLADENPIKQAVIRAI
NNRNRTFVNGTQRYFAEVLANNIYLRAKKENLNTDKISFDYFGIPTIGNGRGIAEIRQLY
EKVDSDIQAYAKGDKPQASYSHLIDAMLAFCIAADEHRNDGSIGLEIDKNYSLYPLDKNT
GEVFTKDIFSQIKITDNEFSDKKLVRKKAIEGFNTHRQMTRDGIYAENYLPILIHKELNE
VRKGYTWKNSEEIKIFKGKKYDIQQLNNLVYCLKFVDKPISIDIQISTLEELRNILTTNN
IAATAEYYYINLKTQKLHEYYIENYNTALGYKKYSKEMEFLRSLAYRSERVKIKSIDDVK
QVLDKDSNFIIGKITLPFKKEWQRLYREWQNTTIKDDYEFLKSFFNVKSITKLHKKVRKD
FSLPISTNEGKELVKRKTWDNNFIYQILNDSDSRADGTKPFIPAFDISKNEIVEAIIDSF
TSKNIFWLPKNIELQKVDNKNIFAIDTSKWFEVETPSDLRDIGIATIQYKIDNNSRPKVR
VKLDYVIDDDSKINYFMNHSLLKSRYPDKVLEILKQSTIIEFESSGFNKTIKEMLGMKLA
GIYNETSNN
Q73QW6
Treponema denticola Cas9
(SEQ ID NO: 47)
MKKEIKDYFLGLDVGTGSVGWAVTDTDYKLLKANRKDLWGMRCFETAETAEVRRLHRGAR
RRIERRKKRIKLLQELFSQEIAKTDEGFFQRMKESPFYAEDKTILQENTLFNDKDFADKT
YHKAYPTINHLIKAWIENKVKPDPRLLYLACHNIIKKRGHFLFEGDFDSENQFDTSIQAL
FEYLREDMEVDIDADSQKVKEILKDSSLKNSEKQSRLNKILGLKPSDKQKKAITNLISGN
KINFADLYDNPDLKDAEKNSISFSKDDFDALSDDLASILGDSFELLLKAKAVYNCSVLSK
VIGDEQYLSFAKVKIYEKHKTDLTKLKNVIKKHFPKDYKKVFGYNKNEKNNNNYSGYVGV
CKTKSKKLIINNSVNQEDFYKFLKTILSAKSEIKEVNDILTEIETGTFLPKQISKSNAEI
PYQLRKMELEKILSNAEKHFSFLKQKDEKGLSHSEKIIMLLTFKIPYYIGPINDNHKKFF
PDRCWVVKKEKSPSGKTTPWNFFDHIDKEKTAEAFITSRTNFCTYLVGESVLPKSSLLYS
EYTVLNEINNLQIIIDGKNICDIKLKQKIYEDLEKKYKKITQKQISTFIKHEGICNKTDE
VIILGIDKECTSSLKSYIELKNIFGKQVDEISTKNMLEEIIRWATIYDEGEGKTILKTKI
KAEYGKYCSDEQIKKILNLKFSGWGRLSRKFLETVTSEMPGFSEPVNIITAMRETQNNLM
ELLSSEFTFTENIKKINSGFEDAEKQFSYDGLVKPLFLSPSVKKMLWQTLKLVKEISHIT
QAPPKKIFIEMAKGAELEPARTKTRLKILQDLYNNCKNDADAFSSEIKDLSGKIENEDNL
RLRSDKLYLYYTQLGKCMYCGKPIEIGHVFDTSNYDIDHIYPQSKIKDDSISNRVLVCSS
CNKNKEDKYPLKSEIQSKQRGFWNFLQRNNFISLEKLNRLTRATPISDDETAKFIARQLV
ETRQATKVAAKVLEKMFPETKIVYSKAETVSMFRNKFDIVKCREINDFHHAHDAYLNIVV
GNVYNTKFTNNPWNFIKEKRDNPKIADTYNYYKVFDYDVKRNNITAWEKGKTIITVKDML
KRNTPIYTRQAACKKGELFNQTIMKKGLGQHPLKKEGPFSNISKYGGYNKVSAAYYTLIE
YEEKGNKIRSLETIPLYLVKDIQKDQDVLKSYLTDLLGKKEFKILVPKIKINSLLKINGF
PCHITGKTNDSFLLRPAVQFCCSNNEVLYFKKIIRFSEIRSQREKIGKTISPYEDLSFRS
YIKENLWKKTKNDEIGEKEFYDLLQKKNLEIYDMLLTKHKDTIYKKRPNSATIDILVKGK
EKFKSLIIENQFEVILEILKLFSATRNVSDLQHIGGSKYSGVAKIGNKISSLDNCILIYQ
SITGIFEKRIDLLKV
U2UMQ6
AcidaminococcusCas12a (Cpf1)
(SEQ ID NO: 48)
MTQFEGFTNLYQVSKTLRFELIPQGKTLKHIQEQGFIEEDKARNDHYKELKPIIDRIYKT
YADQCLQLVQLDWENLSAAIDSYRKEKTEETRNALIEEQATYRNAIHDYFIGRTDNLTDA
INKRHAEIYKGLFKAELFNGKVLKQLGTVTTTEHENALLRSFDKFTTYFSGFYENRKNVF
SAEDISTAIPHRIVQDNFPKEKENCHIFTRLITAVPSLREHFENVKKAIGIFVSTSIEEV
FSFPFYNQLLTQTQIDLYNQLLGGISREAGTEKIKGLNEVLNLAIQKNDETAHIIASLPH
RFIPLFKQILSDRNTLSFILEEFKSDEEVIQSFCKYKTLLRNENVLETAEALFNELNSID
LTHIFISHKKLETISSALCDHWDTLRNALYERRISELTGKITKSAKEKVQRSLKHEDINL
QEIISAAGKELSEAFKQKTSEILSHAHAALDQPLPTTLKKQEEKEILKSQLDSLLGLYHL
LDWFAVDESNEVDPEFSARLTGIKLEMEPSLSFYNKARNYATKKPYSVEKFKLNFQMPTL
ASGWDVNKEKNNGAILFVKNGLYYLGIMPKQKGRYKALSFEPTEKTSEGFDKMYYDYFPD
AAKMIPKCSTQLKAVTAHFQTHTTPILLSNNFIEPLEITKEIYDLNNPEKEPKKFQTAYA
KKTGDQKGYREALCKWIDFTRDFLSKYTKTTSIDLSSLRPSSQYKDLGEYYAELNPLLYH
ISFQRIAEKEIMDAVETGKLYLFQIYNKDFAKGHHGKPNLHTLYWTGLFSPENLAKTSIK
LNGQAELFYRPKSRMKRMAHRLGEKMLNKKLKDQKTPIPDTLYQELYDYVNHRLSHDLSD
EARALLPNVITKEVSHEIIKDRRFTSDKEFFHVPITLNYQAANSPSKFNQRVNAYLKEHP
ETPIIGIDRGERNLIYITVIDSTGKILEQRSLNTIQQFDYQKKLDNREKERVAARQAWSV
VGTIKDLKQGYLSQVIHEIVDLMIHYQAVVVLENLNFGFKSKRTGIAEKAVYQQFEKMLI
DKLNCLVLKDYPAEKVGGVLNPYQLTDQFTSFAKMGTQSGFLFYVPAPYTSKIDPLTGFV
DPFVWKTIKNHESRKHFLEGFDFLHYDVKTGDFILHFKMNRNLSFQRGLPGFMPAWDIVF
EKNETQFDAKGTPFIAGKRIVPVIENHRFTGRYRDLYPANELIALLEEKGIVFRDGSNIL
PKLLENDDSHAIDTMVALIRSVLQMRNSNAATGEDYINSPVRDLNGVCFDSRFQNPEWPM
DADANGAYHIALKGQLLLNHLKESKDLKLQNGISNQDWLAYIQELRN
A0A182DWE3
Lachnospiraceae Cas12a (Cpf1)
(SEQ ID NO: 49)
AASKLEKFTNCYSLSKTLRFKAIPVGKTQENIDNKRLLVEDEKRAEDYKGVKKLLDRYYL
SFINDVLHSIKLKNLNNYISLFRKKTRTEKENKELENLEINLRKEIAKAFKGAAGYKSLF
KKDIIETILPEAADDKDEIALVNSFNGFTTAFTGFFDNRENMFSEEAKSTSIAFRCINEN
LTRYISNMDIFEKVDAIFDKHEVQEIKEKILNSDYDVEDFFEGEFFNFVLTQEGIDVYNA
IIGGFVTESGEKIKGLNEYINLYNAKTKQALPKFKPLYKQVLSDRESLSFYGEGYTSDEE
VLEVFRNTLNKNSEIFSSIKKLEKLFKNFDEYSSAGIFVKNGPAISTISKDIFGEWNLIR
DKWNAEYDDIHLKKKAVVTEKYEDDRRKSFKKIGSFSLEQLQEYADADLSVVEKLKEIII
QKVDEIYKVYGSSEKLFDADFVLEKSLKKNDAVVAIMKDLLDSVKSFENYIKAFFGEGKE
TNRDESFYGDFVLAYDILLKVDHIYDAIRNYVTQKPYSKDKFKLYFQNPQFMGGWDKDKE
TDYRATILRYGSKYYLAIMDKKYAKCLQKIDKDDVNGNYEKINYKLLPGPNKMLPKVFFS
KKWMAYYNPSEDIQKIYKNGTFKKGDMFNLNDCHKLIDFFKDSISRYPKWSNAYDFNFSE
TEKYKDIAGFYREVEEQGYKVSFESASKKEVDKLVEEGKLYMFQIYNKDFSDKSHGTPNL
HTMYFKLLFDENNHGQIRLSGGAELFMRRASLKKEELVVHPANSPIANKNPDNPKKTTTL
SYDVYKDKRFSEDQYELHIPIAINKCPKNIFKINTEVRVLLKHDDNPYVIGIDRGERNLL
YIVVVDGKGNIVEQYSLNEIINNFNGIRIKTDYHSLLDKKEKERFEARQNWTSIENIKEL
KAGYISQVVHKICELVEKYDAVIALEDLNSGFKNSRVKVEKQVYQKFEKMLIDKLNYMVD
KKSNPCATGGALKGYQITNKFESFKSMSTQNGFIFYIPAWLTSKIDPSTGFVNLLKTKYT
SIADSKKFISSFDRIMYVPEEDLFEFALDYKNFSRTDADYIKKWKLYSYGNRIRIFAAAK
KNNVFAWEEVCLTSAYKELFNKYGINYQQGDIRALLCEQSDKAFYSSFMALMSLMLQMRN
SITGRTDVDFLISPVKNSDGIFYDSRNYEAQENAILPKNADANGAYNIARKVLWAIGQFK
KAEDEKLDKVKIAISNKEWLEYAQTSVK
C7NBY4
Leptotrichia buccalis Cas13a (C2c2)
(SEQ ID NO: 50)
MKVTKVGGISHKKYTSEGRLVKSESEENRTDERLSALLNMRLDMYIKNPSSTETKENQKR
IGKLKKFFSNKMVYLKDNTLSLKNGKKENIDREYSETDILESDVRDKKNFAVLKKIYLNE
NVNSEELEVFRNDIKKKLNKINSLKYSFEKNKANYQKINENNIEKVEGKSKRNIIYDYYR
ESAKRDAYVSNVKEAFDKLYKEEDIAKLVLEIENLTKLEKYKIREFYHEIIGRKNDKENF
AKIIYEEIQNVNNMKELIEKVPDMSELKKSQVFYKYYLDKEELNDKNIKYAFCHFVEIEM
SQLLKNYVYKRLSNISNDKIKRIFEYQNLKKLIENKLLNKLDTYVRNCGKYNYYLQDGEI
ATSDFIARNRQNEAFLRNIIGVSSVAYFSLRNILETENENDITGRMRGKTVKNNKGEEKY
VSGEVDKIYNENKKNEVKENLKMFYSYDFNMDNKNEIEDFFANIDEAISSIRHGIVHFNL
ELEGKDIFAFKNIAPSEISKKMFQNEINEKKLKLKIFRQLNSANVFRYLEKYKILNYLKR
TRFEFVNKNIPFVPSFTKLYSRIDDLKNSLGIYWKTPKTNDDNKTKEIIDAQIYLLKNIY
YGEFLNYFMSNNGNFFEISKEIIELNKNDKRNLKTGFYKLQKFEDIQEKIPKEYLANIQS
LYMINAGNQDEEEKDTYIDFIQKIFLKGFMTYLANNGRLSLIYIGSDEETNTSLAEKKQE
FDKFLKKYEQNNNIKIPYEINEFLREIKLGNILKYTERLNMFYLILKLLNHKELTNLKGS
LEKYQSANKEEAFSDQLELINLLNLDNNRVTEDFELEADEIGKFLDFNGNKVKDNKELKK
FDTNKIYEDGENIIKHRAFYNIKKYGMLNLLEKIADKAGYKISIEELKKYSNKKNEIEKN
HKMQENLHRKYARPRKDEKFTDEDYESYKQAIENIEEYTHLKNKVEFNELNLLQGLLLRI
LHRLVGYTSIWERDLRFRLKGEFFENQYIEEIFNFENKKNVKYKGGQIVEKYIKFYKELH
QNDEVKINKYSSANIKVLKQEKKDLYIRNYIAHFNYIPHAEISLLEVLENLRKLLSYDRK
LKNAVMKSVVDILKEYGFVATFKIGADKKIGIQTLESEKIVHLKNLKKKKLMTDRNSEEL
CKLVKIMFEYKMEEKKSEN
P0DOC6
Leptotrichia shahii Cas13a (C2c2)
(SEQ ID NO: 51)
MGNLFGHKRWYEVRDKKDFKIKRKVKVKRNYDGNKYILNINENNNKEKIDNNKFIRKYIN
YKKNDNILKEFTRKFHAGNILFKLKGKEGIIRIENNDDFLETEEVVLYIEAYGKSEKLKA
LGITKKKIIDEAIRQGITKDDKKIEIKRQENEEEIEIDIRDEYINKTLNDCSIILRIIEN
DELETKKSIYEIFKNINMSLYKIIEKIIENETEKVFENRYYEEHLREKLLKDDKIDVILT
NFMEIREKIKSNLEILGFVKFYLNVGGDKKKSKNKKMLVEKILNINVDLTVEDIADFVIK
ELEFWNITKRIEKVKKVNNEFLEKRRNRTYIKSYVLLDKHEKFKIERENKKDKIVKFFVE
NIKNNSIKEKIEKILAEFKIDELIKKLEKELKKGNCDTEIFGIFKKHYKVNFDSKKFSKK
SDEEKELYKIIYRYLKGRIEKILVNEQKVRLKKMEKIEIEKILNESILSEKILKRVKQYT
LEHIMYLGKLRHNDIDMTTVNTDDFSRLHAKEELDLELITFFASTNMELNKIFSRENINN
DENIDFFGGDREKNYVLDKKILNSKIKIIRDLDFIDNKNNITNNFIRKFTKIGTNERNRI
LHAISKERDLQGTQDDYNKVINIIQNLKISDEEVSKALNLDVVFKDKKNIITKINDIKIS
EENNNDIKYLPSFSKVLPEILNLYRNNPKNEPFDTIETEKIVLNALIYVNKELYKKLILE
DDLEENESKNIFLQELKKILGNIDEIDENIIENYYKNAQISASKGNNKAIKKYQKKVIEC
YIGYLRKNYEELFDFSDFKMNIQEIKKQIKDINDNKTYERITVKTSDKTIVINDDFEYII
SIFALLNSNAVINKIRNRFFATSVWLNTSEYQNIIDILDEIMQLNTLRNECITENWNLNL
EEFIQKMKEIEKDFDDFKIQTKKEIFNNYYEDIKNNILTEFKDDINGCDVLEKKLEKIVI
FDDETKFEIDKKSNILQDEQRKLSNINKKDLKKKVDQYIKDKDQEIKSKILCRIIFNSDF
LKKYKKEIDNLIEDMESENENKFQEIYYPKERKNELYIYKKNLFLNIGNPNFDKIYGLIS
NDIKMADAKFLFNIDGKNIRKNKISEIDAILKNLNDKLNGYSKEYKEKYIKKLKENDDFF
AKNIQNKNYKSFEKDYNRVSEYKKIRDLVEFNYLNKIESYLIDINWKLAIQMARFERDMH
YIVNGLRELGIIKLSGYNTGISRAYPKRNGSDGFYTTTAYYKFFDEESYKKFEKICYGFG
IDLSENSEINKPENESIRNYISHFYIVRNPFADYSIAEQIDRVSNLLSYSTRYNNSTYAS
VFEVFKKDVNLDYDELKKKFKLIGNNDILERLMKPKKVSVLELESYNSDYIKNLIIELLT
KIENINDIL
Q0P897
Campylobacter jejuni Cas9
(SEQ ID NO: 52)
MARILAFDIGISSIGWAFSENDELKDCGVRIFTKVENPKTGESLALPRRLARSARKRLAR
RKARLNHLKHLIANEFKLNYEDYQSFDESLAKAYKGSLISPYELRFRALNELLSKQDFAR
VILHIAKRRGYDDIKNSDDKEKGAILKAIKQNEEKLANYQSVGEYLYKEYFQKFKENSKE
FTNVRNKKESYERCIAQSFLKDELKLIFKKQREFGFSFSKKFEEEVLSVAFYKRALKDFS
HLVGNCSFFTDEKRAPKNSPLAFMFVALTRIINLLNNLKNTEGILYTKDDLNALLNEVLK
NGTLTYKQTKKLLGLSDDYEFKGEKGTYFIEFKKYKEFIKALGEHNLSQDDLNEIAKDIT
LIKDEIKLKKALAKYDLNQNQIDSLSKLEFKDHLNISFKALKLVTPLMLEGKKYDEACNE
LNLKVAINEDKKDFLPAFNETYYKDEVTNPVVLRAIKEYRKVLNALLKKYGKVHKINIEL
AREVGKNHSQRAKIEKEQNENYKAKKDAELECEKLGLKINSKNILKLRLFKEQKEFCAYS
GEKIKISDLQDEKMLEIDHIYPYSRSFDDSYMNKVLVFTKQNQEKLNQTPFEAFGNDSAK
WQKIEVLAKNLPTKKQKRILDKNYKDKEQKNFKDRNLNDTRYIARLVLNYTKDYLDFLPL
SDDENTKLNDTQKGSKVHVEAKSGMLTSALRHTWGFSAKDRNNHLHHAIDAVIIAYANNS
IVKAFSDFKKEQESNSAELYAKKISELDYKNKRKFFEPFSGFRQKVLDKIDEIFVSKPER
KKPSGALHEETFRKEEEFYQSYGGKEGVLKALELGKIRKVNGKIVKNGDMFRVDIFKHKK
TNKFYAVPIYTMDFALKVLPNKAVARSKKGEIKDWILMDENYEFCFSLYKDSLILIQTKD
MQEPEFVYYNAFTSSTVSLIVSKHDNKFETLSKNQKILFKNANEKEVIAKSIGIQNLKVF
EKYIVSALGEVTKAEFRQREDFKK
A0A386IRG9
Staphylococcus aureus dCas9
(SEQ ID NO: 53)
MDKKYSIGLATGINSVGWAVITDEYKVPSKKFKVLGNTDRHSIKKNLIGALLFDSGETAE
ATRLKRTARRRYTRRKNRICYLQEIFSNEMAKVDDSFFHRLEESFLVEEDKKHERHPIFG
NIVDEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLFGN
LIALSLGLTPNFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELH
AILRRQEDFYPFLKDNREKIEKILTFRIPYYVGPLARGNSRFAWMTRKSEETITPWNFEE
VVDKGASAQSFIERMTNFDKNLPNEKVLPKHSLLYEYFTVYNELTKVKYVTEGMRKPAFL
SGEQKKAIVDLLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQLKRRRYTGWG
RLSRKLINGIRDKQSGKTILDFLKSDGFANRNFMQLIHDDSLTFKEDIQKAQVSGQGDSL
HEHIANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYDVDA
IVPQSFLKDDSIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLIREVKVITLKS
KLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDF
ATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPK
YSLFELENGRKRMLASAGELQKGNELALPSKYVNFLYLASHYEKLKGSPEDNEQKQLFVE
QHKHYLDEIIEQISEFSKRVILADANLDKVLSAYNKHRDKPIREQAENIIHLFTLTNLGA
PAAFKYFDTTIDRKRYTSTKEVLDATLIHQSITGLYETRIDLSQLGGD
P14727
Xanthomonas euvesicatoria
(SEQ ID NO: 54)
MDPIRSRTPSPARELLPGPQPDGVQPTADRGVSPPAGGPLDGLPARRIMSRTRLPSPPAP
SPAFSAGSFSDLLRQFDPSLFNTSLFDSLPPFGAHHTEAATGEWDEVQSGLRAADAPPPT
MRVAVTAARPPRAKPAPRRRAAQPSDASPAAQVDLRTLGYSQQQQEKIKPKVRSTVAQHH
EALVGHGFTHAHIVALSQHPAALGTVAVKYQDMIAALPEATHEAIVGVGKQWSGARALEA
LLTVAGELRGPPLQLDTGQLLKIAKRGGVTAVEAVHAWRNALTGAPLNLTPEQVVAIASH
DGGKQALETVQRLLPVLCQAHGLTPQQVVAIASNGGGKQALETVQRLLPVLCQAHGLIPQ
QVVAIASNSGGKQALETVQRLLPVLCQAHGLTPEQVVAIASNGGGKQALETVQRLLPVLC
QAHGLTPEQVVAIASNIGGKQALETVQALLPVLCQAHGLTPEQVVAIASNIGGKQALETV
QALLPVLCQAHGLTPEQVVAIASNIGGKQALETVQALLPVLCQAHGLTPEQVVAIASHDG
GKQALETVQRLLPVLCQAHGLTPEQVVAIASHDGGKQALETVQRLLPVLCQAHGLTPQQV
VAIASNGGGKQALETVQRLLPVLCQAHGLTPEQVVAIASNSGGKQALETVQALLPVLCQA
HGLTPEQVVAIASNSGGKQALETVQRLLPVLCQAHGLTPEQVVAIASHDGGKQALETVQR
LLPVLCQAHGLTPEQVVAIASHDGGKQALETVQRLLPVLCQAHGLTPEQVVAIASHDGGK
QALETVQRLLPVLCQAHGLTPQQVVAIASNGGGRPALETVQRLLPVLCQAHGLTPEQVVA
IASHDGGKQALETVQRLLPVLCQAHGLTPQQVVAIASNGGGRPALESIVAQLSRPDPALA
ALTNDHLVALACLGGRPALDAVKKGLPHAPALIKRTNRRIPERTSHRVADHAQVVRVLGF
FQCHSHPAQAFDDAMTQFGMSRHGLLQLFRRVGVTELEARSGTLPPASQRWDRILQASGM
KRAKPSPTSTQTPDQASLHAFADSLERDLDAPSPMHEGDQTRASSRKRSRSDRAVTGPSA
QQSFEVRVPEQRDALHLPLSWRVKRPRTSIGGGLPDPGTPTAADLAASSTVMREQDEDPF
AGAADDFPAFNEEELAWLMELLPQ
B2SU53
Xanthomonas oryzae pv. Oryzae
(SEQ ID NO: 55)
MDPIRSRTPSPARELLPGPQPDRVQPTADRGGAPPAGGPLDGLPARRTMSRTRLPSPPAP
SPAFSAGSFSDLLRQFDPSLLDTSLLDSMPAVGIPHTAAAPAECDEVQSGLRAADDPPPT
VRVAVTAARPPRAKPAPRRRAAQPSDASPAAQVDLRTLGYSQQQQEKIKPKVGSTVAQHH
EALVGHGFTHAHIVALSRHPAALGTVAVKYQDMIAALPEATHEDIVGVGKQWSGARALEA
LLTVAGELRGPPLQLDTGQLVKIAKRGGVTAVEAVHASRNALTGAPLNLTPAQVVAIASN
NGGKQALETVQRLLPVLCQAHGLTPAQVVAIASHDGGKQALETMQRLLPVLCQAHGLPPD
QVVAIASNIGGKQALETVQRLLPVLCQAHGLTPDQVVAIASHGGGKQALETVQRLLPVLC
QAHGLTPDQVVAIASHDGGKQALETVQRLLPVLCQAHGLTPDQVVAIASNGGGKQALETV
QRLLPVLCQAHGLTPDQVVAIASNGGKQALETVQRLLPVLCQAHGLTPDQVVAIASHDGG
KQALETVQRLLPVLCQTHGLTPAQVVAIASHDGGKQALETVQQLLPVLCQAHGLTPDQVV
AIASNIGGKQALATVQRLLPVLCQAHGLTPDQVVAIASNGGGKQALETVQRLLPVLCQAH
GLTPDQVVAIASNGGGKQALETVQRLLPVLCQAHGLTQVQVVAIASNIGGKQALETVQRL
LPVLCQAHGLTPAQVVAIASHDGGKQALETVQRLLPVLCQAHGLTPDQVVAIASNGGGKQ
ALETVQRLLPVLCQAHGLTQEQVVAIASNNGGKQALETVQRLLPVLCQAHGLTPDQVVAI
ASNGGGKQALETVQRLLPVLCQAHGLTPAQVVAIASNIGGKQALETVQRLLPVLCQDHGL
TLAQVVAIASNIGGKQALETVQRLLPVLCQAHGLTQDQVVAIASNIGGKQALETVQRLLP
VLCQDHGLTPDQVVAIASNIGGKQALETVQRLLPVLCQDHGLTLDQVVAIASNGGKQALE
TVQRLLPVLCQDHGLTPDQVVAIASNSGGKQALETVQRLLPVLCQDHGLTPNQVVAIASN
GGKQALESIVAQLSRPDPALAALTNDHLVALACLGGRPAMDAVKKGLPHAPELIRRVNRR
IGERTSHRVADYAQVVRVLEFFQCHSHPAYAFDEAMTQFGMSRNGLVQLFRRVGVTELEA
RGGTLPPASQRWDRILQASGMKRAKPSPTSAQTPDQASLHAFADSLERDLDAPSPMHEGD
QTGASSRKRSRSDRAVTGPSAQHSFEVRVPEQRDALHLPLSWRVKRPRTRIGGGLPDPGT
PIAADLAASSTVMWEQDAAPFAGAADDFPAFNEEELAWLMELLPQSGSVGGTI
A0A158RFF2
Gremmeniella abietina
(SEQ ID NO: 56)
INPWFLTGFIDGEGCFRISVTKINRAIDWRVQLFFQINLHEKDRALLESIKDYLKVGKIH
ISGKNLVQYRIQTFDELTILIKHLKEYPLVSKKRADFELFNTAHKLIKNNEHLNKEGINK
LVSLKASLNLGLSESLKLAFPNVISATRLTDFTVNIPDPHWLSGFASAEGCFMVGIAKSS
ASSTGYQVYLTFILTQHVRDENLMKCLVDYFNWGRLARKRNVYEYQVSKFSDVEKLLSFF
DKYPILGEKAKDLQDFCSVSDLMKSKTHLTEEGVAKIRKIKEGMNRG
Q94AD9
Arabidopsis thaliana.
(SEQ ID NO: 57)
MRTPMSDTQHVQSSLVSIRSSDKIEDAFRKMKVNETGVEELNPYPDRPGERDCQFYLRTG
LCGYGSSCRYNHPTHLPQDVAYYKEELPERIGQPDCEYFLKTGACKYGPTCKYHHPKDRN
GAQPVMENVIGLPMRLGEKPCPYYLRTGTCRFGVACKFHHPQPDNGHSTAYGMSSFPAAD
LRYASGLTMMSTYGTLPRPQVPQSYVPILVSPSQGFLPPQGWAPYMAASNSMYNVKNQPY
YSGSSASMAMAVALNRGLSESSDQPECRFFMNTGTCKYGDDCKYSHPGVRISQPPPSLIN
PFVLPARPGQPACGNFRSYGFCKFGPNCKFDHPMLPYPGLTMATSLPTPFASPVTTHQRI
SPTPNRSDSKSLSNGKPDVKKESSETEKPDNGEVQDLSEDASSP

In a specific embodiment, the Cas9 domain comprises the amino acid sequence at least 50%, 55%, 60%, 65%, 70%, 75%, 80%, 85%, 90%, 91%, 92%, 93%, 94%, 95%, 96%, 97%, 98%, or 100% identical to the amino acid sequence of SEQ ID NO:34, as exemplified in the studies that follow.

The compositions and fusion proteins of the disclosure may include any other functional domains as appropriate for an intended use. In one non-limiting embodiment, the composition or fusion protein may further comprise a localization domain. Any suitable localization domain can be used, including but not limited to any nuclear localization domain. In one non-limiting embodiment, the localization domain may comprises the amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:35 (residues in parentheses are optional). In one embodiment, the optional residues are present. In another embodiment, the optional residues may be absent, in whole or in part.

SEQ ID NO: 35
(YPYDVPDYASLGSGS)PKKKRKVEDPKKKRKV
(DGIGSGSNGSSGSATNFSLLKQAGDVEENPGP)

In a further non-limiting embodiment, the composition or fusion protein may further comprise a detectable domain. Any detection domain can be used, such as a detectable polypeptide domain, as deemed appropriate for an intended use, including but not limited to any fluorescent or luminescent protein or detectable fragment thereof. In one non-limiting embodiment, the detectable domain may comprise the amino acid sequence at least 50%, 55%, 60%, 65%, 70%, 75%, 80%, 85%, 90%, 91%, 92%, 93%, 94%, 95%, 96%, 97%, 98%, 99%, or 100% identical along the length of SEQ ID NO:36.

SEQ ID NO: 36
MVSKGEEDNMAIIKEFMRFKVHMEGSVNGHEFEIEGEGEGRPYEGTQTA
KLKVIKGGPLPFAXAMILSPQFMYGSKAYVKHPADIPDYLKLSFPEGFK
TNERVMNFEDGGVVIVTQDSSLQDGEFIYKVKLRGINFPSDGPVMQKKT
MGTNEASSERMYPEDGALKGEIKQRLKLKDGGHYDAEVKITYKAKKPVQ
LPGAYNVNIKLDITSHNEDYTIVEQYERAEGRHSTGGMDELYK

In another embodiment, the fusion protein comprises the amino acid sequence at least 50%, 55%, 60%, 65%, 70%, 75%, 80%, 85%, 90%, 91%, 92%, 93%, 94%, 95%, 96%, 97%, 98%, or 100% identical to the amino acid sequence SEQ ID NO:37 or SEQ ID NO:38, which are exemplified in the studies described herein.

(SEQ ID NO: 37)
MINEIKKNAQERMDETVEQLKNELSKVRIGGGGTEERRLELAKQVVFAANRALIRVRTIALEAAMRLR
MLGSDKEVNKRDISQALEEIEKLTKVAAKKIKEVLEAKIKELREVMAVN
SGGGGSRGGGSGGGGSGGGGSGGGGSGGGG
MDKKYSIGLAIGINSVGWAVITDEYKVPSKKFKVLGNIDRHSIKKNLIGALLFDSGETAEATRLKRTA
RRRYTRRKNRICYLQEIFSNEMAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFIQLVQTYNQLFEENPINAS
GVDAKAILSARLSKSRRLENLIAQLPGEKKNGLFGNLIALSLGLIPNFKSNFDLAEDAKLQLSKDTYD
DDLDNLLAQIGDQYADLFLAAKNLSDAILLSDILRVNTEITKAPLSASMIKRYDEHHQDLILLKALVR
QQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAMMTRKSEETITPM
NFEEVVDKGASAQSFIERMINFDKNLPNEKVLPKHSLLYEYFTVYNELTKVKYVTEGMRKPAFLSGEQ
KKAIVDLLFKINRKVIVKQLKEDYFKKIECFDSVEISGVEDRFNASLGTYHDLLKIIKDKDFLDNEEN
EDILEDIVLILTLFEDREMIEERLKTYAHLFDDKVMKQLKRRRYTGMGRLSRKLINGIRDKQSGKTIL
DFLKSDGFANRNFMQLIHDDSLIFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYL
QNGRDMYVDQELDINRLSDYDVDAIVPQSFLKDDSIDNKVLIRSDKNRGKSDNVPSEEVVKKMKNYWR
QLLNAKLITQRKFDNLIKAERGGLSELDKAGFIKRQLVETRQIIKHVAQILDSRMNIKYDENDKLIRE
VKVITLKSKLVSDFRKDFQFYKVREINNYHHAHDAYLNAVVGIALIKKYPKLESEFVYGDYKVYDVRK
MIAKSEQEIGKATAKYFFYSNIMNFFKTEITLANGEIRKRPLIETNGETGEIVMDKGRDFATVRKVLS
MPQVNIVKKTEVQTGGFSKESILPKRNSDKLIARKKDMDPKKYGGFDSPIVAYSVLVVAKVEKGKSKK
LKSVKELLGITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILADANLDKVLS
AYNKHRDKPIREQAENIIHLFTLINLGAPAAFKYFDTTIDRKRYISTKEVLDATLIHQSITGLYETRI
DLSQLGGD
(SEQ ID NO: 38)
MINEIKKNAQERMDEIVEQLKNELSKVRIGGGGIEERRLELAKQVVFAANRALIRVRTIALEAAWRLRMLGSDK
EVNKRDISQALEEIEKLIKVAAKKIKEVLEAKIKELREVMAVN
SGGGGSRGGGSGGGGSGGGGSGGGGSGGGG
MDKKYSIGLAIGINSVGWAVIIDEYKVPSKKEKVLGNIDRHSIKKNLIGALLFDSGETAEAIRLKRIARRRYIRR
KNRICYLQEIFSNEMAKVDDSFEHRLEESELVEEDKKHERHPIEGNIVDEVAYHEKYPTIYHLRKKLVDSIDKAD
LRLIYLALAHMIKERGHFLIEGDLNPDNSDVDKLFIQLVQTYNQLFEENPINASGVDAKAILSARLSKSRRLENL
IAQLPGEKKNGLFGNLIALSLGLIPNEKSNFDLAEDAKLQLSKDIYDDDLDNLLAQIGDQYADLFLAAKNLSDAI
LLSDILRVNIEITKAPLSASMIKRYDEHHQDLILLKALVRQQLPEKYKEIFFDQSKNGYAGYIDGGASQEEFYKF
IKPILEKMDGIEELLVKLNREDLLRKQRIFDNGSIPHQIHLGELHAILRRQEDFYPFLKDNREKIEKILIFRIPY
YVGPLARGNSRFAWMIRKSEEIIIPWNFEEVVDKGASAQSFIERMINFDKNLPNEKVLPKHSLLYEYFIVYNELI
KVKYVIEGMRKPAELSGEQKKAIVDLLFKINRKVIVKQLKEDYFKKIECEDSVEISGVEDRFNASLGIYHDLLKI
IKDKDFLDNEENEDILEDIVLILILFEDREMIEERLKIYAHLFDDKVMKQLKRRRYIGWGRLSRKLINGIRDKQS
GKIILDFLKSDGFANRNFMQLIHDDSLIFKEDIQKAQVSGQGDSLHEHIANLAGSPAIKKGILQIVKVVDELVKV
MGRHKPENIVIEMARENQIIQKGQKNSRERMKRIEEGIKELGSQILKEHPVENTQLQNEKLYLYYLQNGRDMYVD
QELDINRLSDYDVDAIVPQSFLKDDSIDNKVLIRSDKNRGKSDNVPSEEVVKKMKNYWRQLLNAKLITQRKFDNL
IKAERGGLSELDKAGFIKRQLVEIRQIIKHVAQILDSRMNIKYDENDKLIREVKVIILKSKLVSDERKDFQFYKV
REINNYHHAHDAYLNAVVGIALIKKYPKLESEFVYGDYKVYDVRKMIAKSEQEIGKATAKYFFYSNIMNFFKIEI
ILANGEIRKRPLIEINGEIGEIVWDKGRDFAIVRKVLSMPQVNIVKKIEVQIGGESKESILPKRNSDKLIARKKD
WDPKKYGGFDSPIVAYSVLVVAKVEKGKSKKLKSVKELLGIIIMERSSFEKNPIDFLEAKGYKEVKKDLIIKLPK
YSLFELENGRKRMLASAGELQKGNELALPSKYVNFLYLASHYEKLKGSPEDNEQKQLFVEQHKHYLDEIIEQISE
FSKRVILADANLDKVLSAYNKHRDKPIREQAENIIHLFILINLGAPAAFKYFDITIDRKRYISIKEVLDAILIHQ
SITGLYEIRIDLSQLGGD
YPYDVPDYASLGSGSPKKKRKVEDPKKKRKVDGIGSGSNGSSGSAINFSLLKQAGDVEENPGP
MVSKGEEDNMAIIKEFMRFKVHMEGSVNGHEFEIEGEGEGRPYEGIQTAKLKVIKGGPLPFAWDILSPQFMYGSK
AYVKHPADIPDYLKLSEPEGFKWERVMNFEDGGVVIVIQDSSLQDGEFIYKVKLRGINFPSDGPVMQKKIMGWEA
SSERMYPEDGALKGEIKQRLKLKDGGHYDAEVKTTYKAKKPVQLPGAYNVNIKLDITSHNEDYTIVEQYERAEGR
HSTGGMDELYK

In one embodiment, the highlighted residues of SEQ ID NO:37 and 38 are not modified.

In another embodiment, the composition or fusion protein is bound to a scaffold, including but not limited to a nanoparticle, virus-like particle (VLP), or other polypeptide scaffold. In embodiments where the composition is a fusion protein, the fusion protein may be further covalently linked to be expressed as part of a polypeptide scaffold. Alternatively, the composition or fusion protein may be linked to the scaffold via any suitable means, as will be apparent to those of skill in the art based on the teachings herein. Any suitable nanoparticle, VLP, or other polypeptide scaffold may be used as deemed appropriate for an intended use.

In another aspect the disclosure provides nucleic acids encoding the polypeptide of any embodiment or combination of embodiments of the disclosure. The nucleic acid sequence may comprise single stranded or double stranded RNA or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded polypeptide, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the disclosure.

In a further aspect, the disclosure provides expression vectors comprising the nucleic acid of any aspect of the disclosure operatively linked to a suitable control sequence. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence.

Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In another aspect, the disclosure provides host cells that comprise the nucleic acids or expression vectors (i.e.: episomal or chromosomally integrated) disclosed herein, wherein the host cells can be either prokaryotic or eukaryotic. The cells can be transiently or stably engineered to incorporate the expression vector of the disclosure, using techniques including but not limited to bacterial transformations, calcium phosphate co-precipitation, electroporation, or liposome mediated-, DEAE dextran mediated-, polycationic mediated-, or viral mediated transfection. In one embodiment, the host cell is stable host cell capable of expressing the polypeptide from the expression vector. In one embodiment, the host cell may also comprise a guide RNA (gRNA) selective for a gene to be activated (for example, a gRNA-encoding nucleic acid; an expression vector comprising a gRNA encoding sequence operatively linked to a suitable control sequence, etc.) This embodiment can, for example, be used in methods of the disclosure that involve culturing host cells under conditions suitable to promote targeting of the gene to be activated with the gRNA and the polypeptide, wherein the polypeptide directs PRC2 disruption at the gene targeted by the gRNA, thus activating the gene. This embodiment is described in more detail below. The host cells may be individual cells, tissues, and/or any may be present within a recombinant non-human organism, including but not limited to Drosophila sp., zebrafish, mice, etc.

A method of producing a polypeptide according to the invention is an additional part of the disclosure. The method comprises the steps of (a) culturing a host according to this aspect of the disclosure under conditions conducive to the expression of the polypeptide, and (b) optionally, recovering the expressed polypeptide. The expressed polypeptide can be recovered from the cell free extract, but preferably they are recovered from the culture medium.

In another aspect, the present disclosure provides pharmaceutical compositions, comprising one or more compositions, nucleic acids, expression vectors, and/or host cells of the disclosure and a pharmaceutically acceptable carrier. The pharmaceutical compositions of the disclosure can be used, for example, in the methods of the disclosure described below. The pharmaceutical composition may comprise in addition to the compositions of the disclosure (a) a lyoprotectant; (b) a surfactant; (c) a bulking agent; (d) a tonicity adjusting agent; (e) a stabilizer; (f) a preservative and/or (g) a buffer.

In some embodiments, the buffer in the pharmaceutical composition is a Tris buffer, a histidine buffer, a phosphate buffer, a citrate buffer or an acetate buffer. The pharmaceutical composition may also include a lyoprotectant, e.g. sucrose, sorbitol or trehalose. In certain embodiments, the pharmaceutical composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the pharmaceutical composition includes a bulking agent, like glycine. In yet other embodiments, the pharmaceutical composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The pharmaceutical composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the pharmaceutical composition additionally includes a stabilizer, e.g., a molecule which, when combined with a protein of interest substantially prevents or reduces chemical and/or physical instability of the protein of interest in lyophilized or liquid form. Exemplary stabilizers include sucrose, sorbitol, glycine, inositol, sodium chloride, methionine, arginine, and arginine hydrochloride.

The compositions, nucleic acids, expression vectors, and/or host cells may be the sole active agent in the pharmaceutical composition, or the composition may further comprise one or more other active agents suitable for an intended use, such as an appropriate gRNA construct (for example, a gRNA-encoding nucleic acid; expression vector comprising a gRNA encoding sequence operatively linked to a suitable control sequence) targeting a gene to be activated, as detailed below.

In another aspect, the disclosure provides kits comprising:

(a) an active composition/fusion protein, nucleic acid, expression vector, host cell, and/or pharmaceutical composition of any embodiment or combination of embodiments disclosed herein; and

(b) a control composition, nucleic acid, expression vector, host cell, and/or pharmaceutical composition that is identical to the active composition, the active nucleic acid, the active expression vector, host cell, and/or pharmaceutical composition, except that the EB domain is inactive (i.e.: does not bind to EED), and/or the control nucleic acid encodes an inactive EB domain.

The kit can be used for any suitable purpose, including but not limited to promote single gene activation as described herein, and verify specificity of targeting via use of the control. Any inactive EB control can be used as appropriate for an intended use. In one non-limiting embodiment, the inactive EB domain comprises the amino acid sequence at least 50%, 55%, 60%, 65%, 70%, 75%, 80%, 85%, 90%, 91%, 92%, 93%, 94%, 95%, 96%, 97%, 98%, or 100% identical to the amino acid sequence of SEQ ID NO:13, wherein the highlighted residues are modified to polar or charged amino acids (i.e., K, R, H, G, S, T, C, Y, N, Q, D, E).

(SEQ ID NO: 13)
MINEIKKNAQERMDETVEQLKNELSKVRIGGGGTEERRLELAKQVVFAAN
RALIRVRTIALEAAWRLRMLGSDKEVNKRDISQALEEIEKLTKVAAKKIK
EVLEAKIKELREVMAVN

In non-limiting embodiments, the inactive EB domain comprises the amino acid sequence at least 50%, 55%, 60%, 65%, 70%, 75%, 80%, 85%, 90%, 91%, 92%, 93%, 94%, 95%, 96%, 97%, 98%, or 100% identical to the amino acid sequence of SEQ ID NO:10, 12, or 39 wherein the highlighted residues are not modified, or are modified to other polar amino acid residues (K, R, H, G, S, T, C, Y, N, Q, D).

>EB15.2NC
(SEQ ID NO: 10)
HMGQRWELALQRFWDYLRWVQTLSEQVQEELLSDKAIEELAALAKETERE
LRNYIAELSKQLTPVAEETKRQLATTLVEVANRLKETMRTIMLELLRYRI
AVNALNGQSTEDLRRNLAENLRKSRDDLLITADKLQRVLAVYQAGALE
>EB22.2NC
(SEQ ID NO: 12)
(SEQ ID NO: 39)
HMINEIKKNAQERMDETVEQLKNELSKVRIGGGGTEERRLELAKQVVEAA
NRALERVRTIALEAAWRLRMLGSDKEVNKRDISQALEEIEKLTKVAAKKI
KEVLEAKIKELREVLEMINEIKKNAQERMDETVEQLKNELSKVRIGGGGT
EERRLELAKQVVEAANRALERVRTIALEAAWRLRMLGSDKEVNKRDISQA
LEEIEKLTKVAAKKIKEVLEAKIKELREVMAVN

In another aspect, the disclosure provides methods for use of the composition, nucleic acid, expression vector, host cell, pharmaceutical composition, or kit of any embodiment or combination of embodiments disclosed herein, for gene activation in a biological cell. As described in the examples that follow, the inventors have discovered that the composition, nucleic acid, expression vector, host cell, pharmaceutical composition, or kit of the disclosure can be used, for example, to direct PRC2 disruption at precise loci using gRNA and by that locally reduce H3K27me3 marks to promote single gene activation. Any gene can be activated using the methods disclosed herein.

As disclosed in the examples that follow, the inventors have discovered that the fusion proteins disclosed herein can be used, for example, to direct PRC2 disruption at precise loci using gRNA and by that locally reduce H3K27me3 marks to promote single gene activation. Such precise control of epigenetic regulation can be used, for example, to treat human diseases or direct cell fate linage free of traditional chemical drugs or DNA manipulation, and as a research tool will for the study of the epigenetic memory of loss of specific H3K27 methyl marks.

The methods may comprise contacting the biological cell in vivo (for example, to treat disease), ex vivo (for example, to treat cells to be placed back into a subject for disease treatment), or in vitro (for example, in research use).

Clustered regularly interspaced short palindromic repeats (CRISPR), the bacterial defense system using RNA-guided DNA cleaving enzymes may comprise directing the CRISPR-associated (Cas) proteins (such as Cas9) to multiple gene targets by providing guide RNA sequences complementary to the target sites. Target sites for CRISPR/Cas9 systems can be found near most genomic loci; the only requirement is that the target sequence, matching the guide strand RNA, is followed by a protospacer adjacent motif (PAM) sequence in either orientation. For Streptococcus pyogenes (Sp) Cas9, this is any nucleotide followed by a pair of guanines (“NGG”).

As used herein, the “gRNA” refers to a guide RNA which in an embodiment is a fusion between the gRNA guide sequence (or CRISPR targeting RNA or crRNA) and the CRISPR nuclease recognition sequence (tracrRNA). It provides both targeting specificity and scaffolding/binding ability for the Cas9. Alternatively, the gRNA may be provided as two separate entities (a tracrRNA and a gRNA guide sequence (i.e., target-specific sequence/crRNA)).

A “target region” refers to the region of the target gene which is targeted by the gRNA. The methods may include use of at least one (1, 2, 3, 4, 5, or more) gRNAs, wherein each gRNA targets a different DNA sequence on the target gene. The target DNA sequences may be overlapping. The target sequence or protospacer is followed or preceded by a PAM sequence at an end of the protospacer. Generally, the target sequence is immediately adjacent (contiguous) to the PAM sequence; it is located on the 5′ end of the PAM for SpCas9-like nuclease.

The CRISPR targeting RNA or crRNA refers to the portion of the gRNA guide sequence that binds to the Cas9. It leads the Cas9 to the target sequence so that it may bind and cut the target nucleic acid. It is adjacent the gRNA guide sequence. In embodiments, the crRNA has at least 65 to 77 nucleotides.

In embodiments, the gRNA may comprise a “G” at the 5′ end of its polynucleotide sequence. The presence of a “G” in 5′ is preferred when the gRNA is expressed under the control of the U6 promoter. The gRNAs may be of varying lengths. The gRNA may comprise a gRNA guide sequence of at least 10 nts, at least 11 nts, at least a 12 nts, at least a 13 nts, at least a 14 nts, at least a 15 nts, at least a 16 nts, at least a 17 nts, at least a 18 nts, at least a 19 nts, at least a 20 nts, at least a 21 nts, at least a 22 nts, at least a 23 nts, at least a 24 nts, at least a 25 nts, at least a 30 nts, or at least a 35 nts of a target sequence in the gene target. In embodiments, the “gRNA guide sequence” or “gRNA target sequence” may be least 10 nucleotides long; in some embodiments 10-40 nts long (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nts long). In other embodiments, gRNA guide sequence is between 17-30, 17-22, 10-40, 10-30, 12-30, 15-30, 18-30, or 10-22 nucleotides long.

The number of gRNAs administered to or expressed in a target cell in accordance with the methods of the present invention may be at least 1 gRNA, at least 2 gRNAs, at least 3 gRNAs at least 4 gRNAs, at least 5 gRNAs, at least 6 gRNAs, at least 7 gRNAs, at least 8 gRNAs, at least 9 gRNAs, at least 10 gRNAs, at least 11 gRNAs, at least 12 gRNAs, at least 13 gRNAs, at least 14 gRNAs, at least 15 gRNAs, at least 16 gRNAs, at least 17 gRNAs, or at least 18 gRNAs.

Although a perfect match between the gRNA guide sequence and the DNA sequence on the targeted gene is preferred, a mismatch between a gRNA guide sequence and target sequence on the gene sequence of interest is also permitted as along as it still allows hybridization of the gRNA with the complementary strand of the gRNA target polynucleotide sequence on the targeted gene.

Any gRNA guide sequence can be selected in the target gene, as long as it allows introducing at the proper location, the desired modification(s). Accordingly, the gRNA guide sequence or target sequence of the present invention may be in coding or non-coding regions of the target gene

In one embodiment, the gRNA is encoded by an expression vector and the gRNA encoding sequence is operatively linked to a suitable control sequence. In one embodiment, the sequence encoding the gRNA is within 50-100 base pairs of a TATA box. In one such embodiment, the TATA box is 5′ to the gRNA encoding sequence; in another embodiment, the TATA box is 3′ to the gRNA encoding sequence. As described in the examples that follow, no sequence or structural gRNA requirements were found with regard to TATA box proximity, and no functional PAM specificity was found with regard to TATA box region.

In one embodiment, the methods comprise

(a) providing a host cell of the present disclosure, which comprises an expression vector and/or nucleic acid of the disclosure;

(b) contacting the host cell with a guide RNA (gRNA) selective for a gene to be activated, including but not limited to adding the gRNA at the time of gene activation, or providing host cells that express the gRNA (including but not limited to host cells transfected with a viral construct or transiently or stably transfected with a plasmid, in each case having an appropriate promoter (including but not limited to U6) controlling gRNA expression); and

(c) culturing the cells under conditions suitable to promote expression of the polypeptide in the host cell, wherein the polypeptide directs PRC2 disruption at the gene targeted by the gRNA, thus activating the gene.

In another embodiment, the methods comprise

(a) transfecting a host cell with a particle comprising the composition of any embodiment of the disclosure and a guide RNA (gRNA) selective for a gene to be activated; and

(b) culturing the cells under conditions suitable to promote targeting of the gene to be activated with the gRNA and the particle, wherein the composition directs PRC2 disruption at the gene targeted by the gRNA, thus activating the gene.

In one specific embodiment, the methods are used to treat gliobastoma (for example, pediatric glioblastoma), including but not limited to Diffuse Intrinsic Pontine Glioma (DIPG)-17B. In highly lethal pediatric glioblastoma the histone mutation H3.3K27M causes an increase in H3K27me3 at the cell cycle regulator (cyclin dependent kinase inhibitor 2A) CDKN2A locus also known as p16. P16 expression inhibits cyclin dependent kinase 4, which activates the retinoblastoma family of proteins, to block cell cycle from G1 to S. Repression of p16 by hypermethylation (H3K27me3) in DIPG cells prevents block of cell cycle, thereby allowing tumorigenesis. EBdCas9/gRNA targeting p16 in DIPG cells results in p16 transcript and protein expression and consequently, initiation of cell cycle halts from G1 to S phase. DIPG occurs in brainstem, a vital region of the brain, where there are minimal surgical options, limited chemotherapy as well as radiation therapy to provide palliative relief at best. EBdCas9 and its specificity to H3K27me3 p16 targets using gRNA holds great promise for epigenetic therapeutic agent in DIPG cells.

Thus, in one embodiment, the biological cell is present within a subject having glioblastoma, wherein the gene targeted by the gRNA comprises the p16 gene, and wherein the gene activation serves to treat the glioblastoma.

In another specific embodiment, the methods are used for research applications targeting gene activation, epigenetic remodeling, and chromatin architecture. In one such embodiment, a nucleic acid encoding a fusion protein of the disclosure is operatively linked to a metallothionein (MT) promoter region in an appropriate expression vector for use in Drosophila Melanogaster, thereby permitting induced fusion protein expression upon heavy metal binding to the MT promoter region. Such conditional induction of the fusion proteins of the disclosure can be used in embryogenesis, development and tissue regeneration for research application targeting gene activation, epigenetic remodeling, and chromatin architecture. Additional alternative inducible promoters systems can be used, including but not limited to those listed in Table 1.

TABLE 1

| system | Target | GeneSwitch | Q system | Tet-On | PRExpress |
|---|---|---|---|---|---|
| Promoter | UAS | UAS | QUAS | tetO | poly-PRE- |
| | | | | | hsp70 |
| Activator | GAL4 | GLp65 | QF | rtTA | heat shock |
| Inducer | heat shock | RU486 | Quinic | Doxy- | heat shock |
| | 19° C. to | | acid | cycline | 25° C. to |
| | 30° C. | | | | 37° C. |

In another specific research embodiment, a nucleic acid encoding a fusion protein of the disclosure is operatively linked to a heat shock promoter region in an appropriate expression vector for use in zebrafish, thereby permitting induced fusion protein expression upon heat shock. Such conditional induction of the fusion proteins of the disclosure can be used in the zebrafish model to study embryogenesis, development and tissue regeneration for research application targeting gene activation, epigenetic remodeling, and chromatin architecture. Additional alternative inducible promoters systems can be used, including but not limited to those listed in Table 2.

TABLE 2

| | | | | |
|---|---|---|---|---|
| system | Target | photoreceptor | Tet-On | heatshock |
| Promoter | UAS | UAS/CRY2 | tetO | HSP70 |
| Activator | GAL4 | GAL4 | rtTA | heat shock |
| Inducer | heat shock | Blue light | Doxycycline | heat shock |
| | 19° C. to 30° C. | | | 25° C. to 37° C. |

### EXAMPLES

### Abstract

Bifurcations in cell fates are controlled through epigenetic modifications. Particularly, broad H3K27me3 marks are known to repress developmental genes, however the precise chromatin locations of functional H3K27me3 marks are not yet known. To identify the functional H3K27me3 loci in promoter regions, we fused a computationally designed protein, EED binder (EB) that competes over EZH2 and thereby disrupts PRC2 function, to dCas9 to direct PRC2 inhibition at a precise locus using gRNA. Here we show that EBdCas9 identifies PRC2 requirement in a single nucleosome to repress transcription of the downstream gene. In the case of Tbx18 we reveal the mechanism: the distant, upstream TATAbox is normally silenced by PRC2 complex. Furthermore, we show that the earliest cell fate bifurcation in developing animal requires PRC2 based repressive epigenetic marks only in a very narrow chromatin region upstream of two genes, GATA3 and Cdx2. EBdCas9 is sufficient to transdifferentiate iPSC to human trophoectoderm when directed with gRNA to specific 100 bp DNA regions. EBdCas9 tool is broadly applicable for epigenetic regulation of single locus to pinpoint and regulate PRC2 dependent critical marks for control of gene expression.

### Introduction

A central question in epigenetics and developmental biology is the role of specific histone 3 lysine 27 methylation (H3K27me3) marks in cell fate decisions. PRC2 is an evolutionarily conserved, repressive H3K27me3 methyltransferase complex that plays a key role in developmental transitions. Broad upstream regions of developmental genes are decorated with H3K27me3 marks, however it is not known which, if any single nucleosomes require H3K27me3 marks for gene repression and cell fate determination.

The two main complexes involved in Polycomb based repression are PRC1 and PRC2. PRC1 catalyzes monoubiquitylation of Lys 119 of histone H2A (H2AK119ub) while PRC2 catalyzing the mono-, di- and trimethylation of Lys27 of histone H3 (H3K27me1/me2/me3). It is not known if any specific H3K27me3 marked nucleosomes are critical for function or if the broad 2.5 kb region is essential for gene repression. This has been challenging to address since previous genetic methods have eliminated all H3K27me3 marks, without precision.

There is no current way to inhibit PRC2 function at a specific genomic locus, and precisely, at single nucleosome to test which H3K27me3 marks play key roles in the repression of transcription. We have generated a computer designed protein that binds EED and thereby competes over EZH2 localized activity (EB). Here, by fusing the designed PRC2 inhibitor EB to dCas9, we enable probing H3K27me3 function in precise gene loci in the natural biological context. We show that EBdCas9/gRNA is able to upregulate genes of interest, remodel targeted sites' epigenetics, promote epigenetic memory. We reveal the mechanism showing that PRC2 action represses a distant TATAbox region. Additionally, we applied EBdCas9 to address the biological question of developmental epigenetic control of bifurcation decisions between ICM and TE as it dependent on H3K27me3 marks. We now identify the precise location of H3K27me3 marks that are critical for TE differentiation.

### Results:

### EBdCas9/gRNA Activates TBX18 Transcription

The catalytic and substrate recognition functions of PRC2, mediated by the SET domain containing EZH2 subunit and the tri-methyl lysine binding EED subunit, respectively, are coupled by binding of the N-terminal helix of EZH2 to an extended groove on EED. We previously generated and characterized a computationally designed protein that binds to the EZH2 binding site on EED³³. The designed EED binder protein (EB) is stable, binds to EED with subnanomolar affinity, forms tight complexes with EED, reduces EZH2, and JARID2 global levels, and exhibits a significant genome wide reduction of H3K27me3 repressive marks in promoter regions³³. Conditionally expressed EB showed that PRC2 is essential at primed ESC stages but dispensable in early naïve ESC stages. As a control, we created an EED binder negative control (NC), where two amino acid mutations: F47E and I54E on the EED binding interface abolish binding to EED³³.

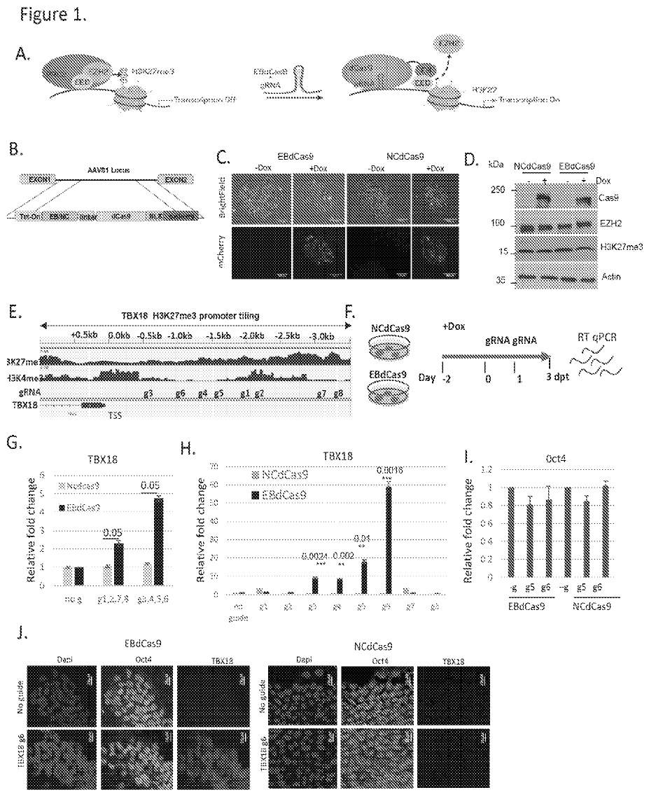

To target EB to specific chromatin locus to test its functionality in precise regions we fused EB to dCas9. EBdCas9 together with targeted guide RNA (gRNA) will allow us, for the first time, to disrupt PRC2 at the local level and identify which H3K27me3 marks at precise loci, if any are required for control of gene expression of the targeted gene of interest (FIG. 1A). To generate EBdCas9 protein, we fused EB into the AAVS1-TREG inducible promotor of dCas9-NLS-mCherry™ plasmid³⁸ (FIG. 1B). Similarly, we fused NC control to dCas9 to critically distinguish between dCas9 unspecific effects in chromatin and EB specific effects on the histone modifications in the loci and transcription. A 30 aa residue 6×5 (SGGGG) (SEQ ID NO:14) linker was inserted between EB or EBNC and dCas9 for free mobility and permissive binding action once EB/NCdCas9/gRNA is bound to targeted DNA. The EB-linker-dCas9-NLS-mCherry (EBdCas9) and EBNC-linker-dCas9-NLS-mCherry™ (NCdCas9) constructs were transformed to iPSC (WTC) using TALENS to enforce recombinant homology at the safe harbor locus, AAVS1 site on chromosome 19. Following antibiotic selection, the lines were validated for EBdCas9 and NCdcas9 mCherry™ expression with or without Dox induction where no leakiness was observed and stem cell morphology was maintained (FIG. 1C). Unlike EB, that causes global EZH2 and H3K27me3 reduction in hESC³³, WTC cells are not affected by EBdCas9 when induced without gRNA, and EZH2 and H3K27me3 levels remain the same between induced and uninduced EBdCas9 (FIG. 1D). EBdCas9 and NCdCas9 transcript expression was found to be 50×lower compared to EB or NC suggesting that construct based off-target effects may be minimal with the EBdCas9 construct (data not shown). It is also plausible that EBdCas9 fusion may comprise conformational steric hindrance effects that do not allow EB to bind promiscuously to EED and therefore no global H3K27me3 or EZH2 reduction is observed.

To identify the effects of targeting EBdCas9 or NCdCas9 to specific chromosomal loci we screened for genes that showed the most H3K27me3 reduction after EB treatment previously tested in ChIPseq™ H3K27me3 EB analysis³³. TBX18, a growth promoting transcription factor of the sinoatrial node T-box 18, required for embryonic development and conversion of working myocytes into sinoatrial cells was observed as a highly significantly upregulated gene with reduced H3K27me3 marks after EB expression and was therefore selected as a candidate locus to analyze the action of EBdCas9 construct. At iPSC stage TBX18 gene shows bivalency, the gene upstream region is simultaneously decorated with both H3K27me3 repressive marks and H3K4me3 active marks. We tiled the TBX18 upstream region with guides to identify the loci sensitive for targeted-locus-activation by using CRISPRscan⁴¹ gRNAs prediction tool (FIG. 1E). EBdCas9 was induced at day −2 using doxycycline and transiently transfected with in vitro synthesized gRNA at day 0 and day 1 and the cells were collected at day 3 (FIG. 1F). To initiate the screens to test which upstream regions more sensitive to EB action and therefore more likely to activate transcription, we performed a combinatorial induction of clusters of guides. Interestingly, while guides 1.9-3.5 kb away from TSS (g1,2,7,8) did not show significant effects in Tbx18 transcription, the guides 0-1.9 kb from TSS (g3,4,5,6) showed 4 fold TBX18 upregulation compared to no guide treatment (FIG. 1G). The control NCdCas9 did not show effects with either groups of guides (FIG. 1G).

To dissect the EB responsive region in a more precise manner, we transfected the cells with each guide individually and analyzed Tbx18 transcriptional increase. Induction of EBdCas9 with the individual TBX18 gRNAs resulted in TBX18 transcript upregulation between 10 fold (gRNA 3 and 4) and 50-60 fold (gRNA 5 and 6) compared to no guide, EBdCas9(g1,2,7,8) or NCdCas9 (FIG. 1H). To understand the rules for gRNA positioning for transcript activation, we analyzed gRNA distribution on TBX18 promoter bivalent region and observed that gRNAs 3-6 (−0.5 kb to −1.5 kb) localized to unique chromatin domain where H3K4me3 marks are depleted and H3K27me3 marks are enriched. Targeted localization of gRNA 3-6 within 1.5 kb of promoter proximity, together with the bivalent marks architecture, we propose keeps TBX18 poised for transcript activation compared to gRNA 1,2,7 and 8 (−1.9 kb to −3.5) that are deficient of these features. To ensure all tiled gRNAs are equally accessible to targeted TBX18 DNA, we used hESC Elf iCas9 cell line (20) to transiently transfected different gRNAs and to analyze DNA accessibility by cutting and indel analysis (data not shown) at targeted site. 7 out pf 8 gRNAs showed indels at targeted site, proving accessibility. To test the specificity of EBdCas9, we monitored OCT4 transcript gene expression and observed no significant changes in Oct4 in TBX18 guided samples (FIG. 1I), hence Tbx18 upregulation is not a secondary effect of differentiation. TBX18 protein over expression was detected with EBdCas9/g6, but not with NCdCas9/g6 (FIG. 1J). We conclude that EBdCas9, but not NCdCas9 is able to activate TBX18 gene expression at precise loci.

### EBdCas9 Precisely Remodels TBX18 Epigenetic Marks and Retains Epigenetic Memory

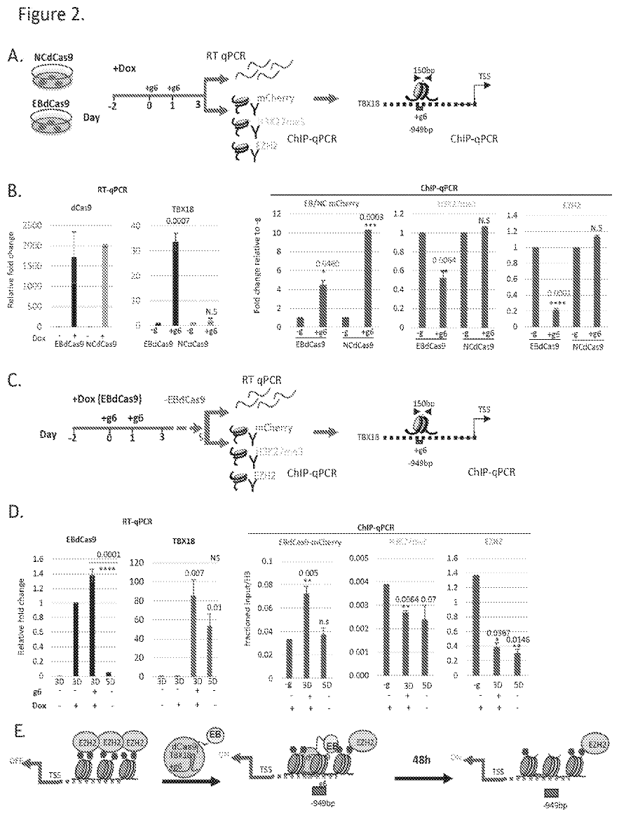

To dissect the mechanism of EBdCas9 at precise genomic locus, PIXUL-ChIP™ was used to analyze the epigenetic landscape of TBX18 g6 targeted region. The primer pair for this analysis is directly by guide 6 locus and produce an amplicon of 150 bp. WTC EBdCas9 or NCdCas9 were induced using doxycycline followed by 2 gRNA transfections with TBX18 g6 RNA (g6) and harvested on day 3 (FIG. 2A). RTqPCR of EBdCas9 showed 30 fold increase of TBX18 transcript compare to NCdCas9 and no significant change for dCas9 expression (FIG. 2B). The ChIPqPCR™ assay confirms that both EBdCas9 and NCdCas9 are recruited to guide 6 locus using mCherry™ antibody, however, EBdCas9 but not NCdCas9 results in reduction of H3K27me3 marks and EZH2 at guide 6 specific locus (FIG. 2C). This data shows that EBdCas9 is able to disrupt EED-EZH2 interaction at precise locus which also results in the depletion of H3K27me3 marks at this site. To learn whether depleting of H3K27me3 marks and EZH2 by EBdCas9 retains epigenetic memory we repeated the assay as before but continue to grow the cells for 2 additional days free of EBdCas9 or gRNA and harvested at day 5 (FIG. 2C). RT-qPCR of EBdCas9 transcript shows upregulation at 3 days post transfection (dpt) and complete disappearance by day 5, however, TBX18 transcript shows 80 fold increase at day 3 and 50 fold increase at day 5 which is an indicative of transcript memory (FIG. 2D). To validate TBX18 transcript memory is a result of epigenetic memory we preformed PIXUL-ChIP™ on 3 and 5 days samples; ChIPqPCR™ assay showed the recruitment of EBdCas9 to guide 6 locus at days 3 but gone in day 5, however, both H3K27me3 and EZH2 showed depletion at day 3 that was also observed in day 5 (FIG. 2D). This data shows that EBdCas9 not only remodels the epigenome but also leads to epigenetic memory (FIG. 2E).

### EBdCas9 Causes Epigenetic Neighborhood Spreading and Reveals Distant TATAbox

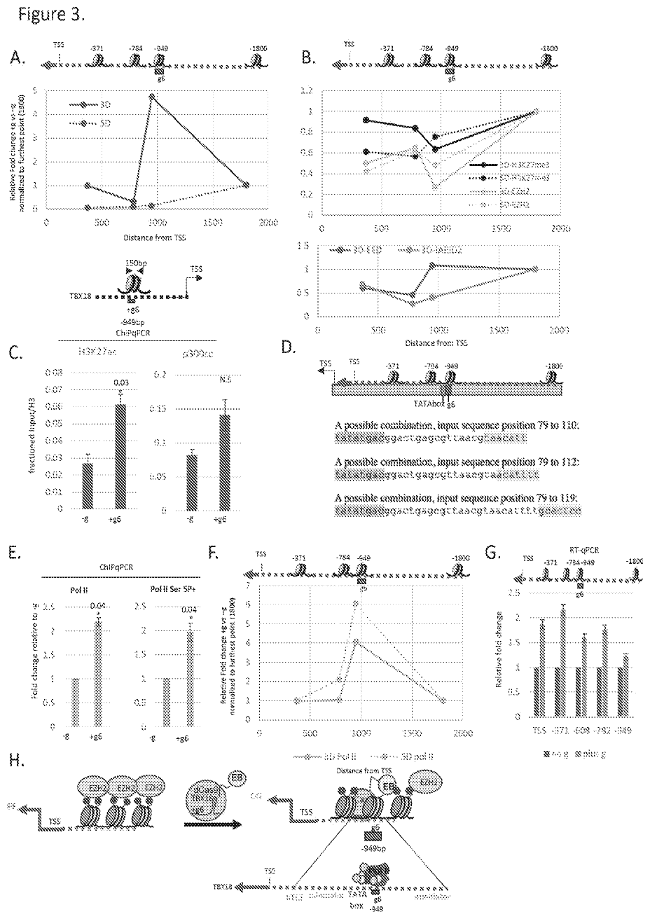

To learn whether (i) EBdCas9/TBX18g6 epigenetic marks de-repression is limited to guide 6 local section, (ii) the marks are spreading to the neighborhood region, or whether (iii) the marks are enhanced or reversed upon epigenetic memory, we tiled TBX18 genomic area with different primer sets. As expected, 3D mCherry™ is solely localized to g6 region but not at 5D (FIG. 3A). H3K27me3 and EZH2 marks shows spreading to the neighborhood region at 3D and these marks are further depleted at 5D (FIG. 3B). Other PRC2 components that showed spreading at the neighborhood regions at 3D are JARID 2 and SUZ12 (FIG. 3B). EED on the other hand, binds to EBdCas9 and remain balanced at guide 6, however is depleted at other regions. This data suggests that not only the epigenetic marks are spreading towards TSS but PRC2 at the neighborhood regions are also disrupted. To validate that targeted depleted marks are supplemented with activation marks, we performed ChIPqPCR™ using H3K27ac and p300 and observed recruitment to TBX18 g6 locus (FIG. 3C). Since TBX18 g6 targeted site was so prominent to transcript activation, epigenetic remodeling and epigenetic memory, we used Element Navigation Tool™⁴³ for detection of core promoter elements: when given TBX18 promoter region (˜1000 bp) reveals a possible combination of TATAbox 50 bp downstream and mammalian initiator factor ˜70 bp downstream of TBX18 g6 locus (FIG. 3D). As targeted de-repressed PRC2 by EBdCas9 reveals a masked far TATAbox for TBX18 gene activation, we hypothesized that RNA pol II may be recruited for TBX18 g6 site. ChIPqPCR™ using RNA Pol II CTD and RNA Pol II Ser 5 phosphorylated (Pol II pause) validated their recruitment to TBX18 g6 locus (FIG. 1E). Furthermore, RNA pol II CTD neighborhood spreading was restricted to TBX18 g6 site at 3D and those marks were further enhanced at 5D (FIG. 3F). To validate that TBX18 mRNA (or 5′UTR) is transcribed from guide 6 region, we RT-qPCR this locus only and observed amplification of tiled neighborhood regions compared to no guide (FIG. 1G). Overall, we can conclude that EBdCas9 together with TBX18 g6 was able to identify a PRC2 nucleated region which was repressing far TATAbox site to silence TBX18 gene expression (FIG. 1H).

### EBdCas9 Activates CDKN2A by Epigenetic Remodeling

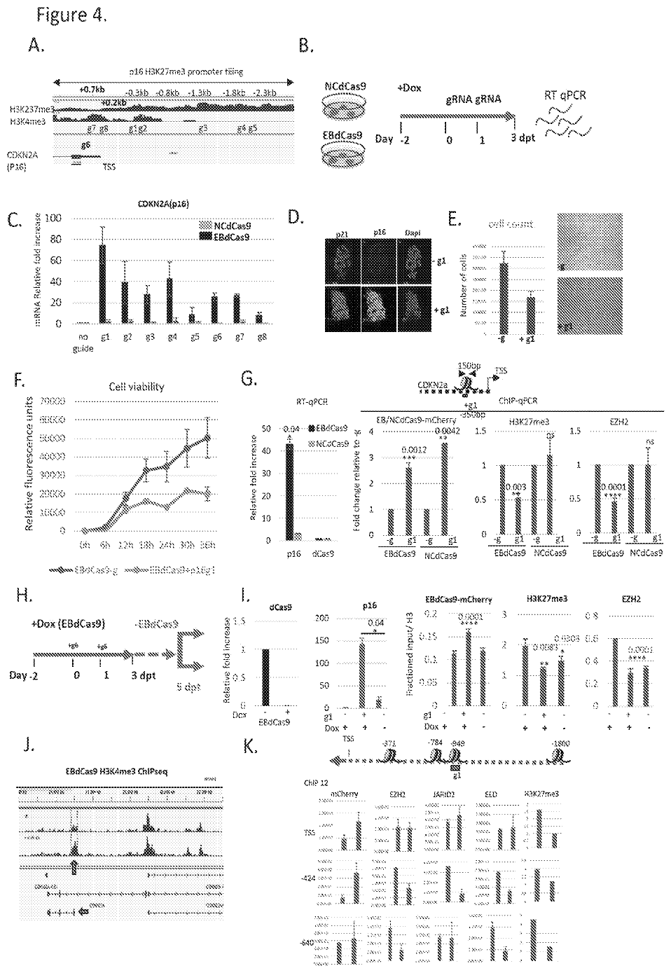

To identify the effects of EBdCas9 on other functional H3K27me3 prior to gene activation, we explore CDKN2A gene (p16). P16, is a critical regulator of cell division and a tumor suppressor, that inhibits cyclin D-dependent protein kinase activity and by that reduce G1-S transition^{44, 45}. In rapidly dividing cells, such as in diffuse intrinsic pontine glioma (DIPG), p16 is repressed due to hypermethylation at the promoter area⁴⁶. Since iPSC WTC EBdCas9 are also rapidly dividing cells, we hypothesize that it could serve as a model and therefore provide insights into the effects of changes in epigenetic regulation in gliomagenesis. Induction of EBdCas9 to p16 promoter area can modulate epigenetic regulation and could suggest new routes for glioma treatment. We tiled the promoter area and gene body of p16 with 8 gRNAs ranging from 0.3 kb to 2.3 kb upstream of TSS and 0.2 kb to 0.7 kb downstream of TSS (FIG. 4A). WTC EBdCas9 or NCdCas9 were induced prior to transient transfection of the gRNAs and followed by cell harvest at 3D for p16 transcript analysis (FIG. 4B). EBdCas9 activated p16 transcript expression on 6 out of the 8 gRNAs, but none were activated by NCdCas9 (FIG. 4C). As observed with TBX18 tiling, gRNAs that are in 0.5 kb-1.5 kb proximity to TSS showed the most p16 transcript activation, as in g1, g2, g3, g4, g6, and g7 ranging from 20-80 fold of increase compared to −g or NCdCas9. However, g5 which is 2.2 kb upstream of TSS or g8 which is 0.1 kb downstream of TSS resulted with less than 10 fold of transcript increase. g8 RNA was deliberately chosen as an internal control as a proof of concept that binding of dCas9 in 0.1 kb proximity to TSS should block transcription free of EB mechanism. To validate p16 transcript in full length and translation we validated p16 protein overexpression using immunofluorescence analysis (FIG. 4D). Since activation of p16 results in halt of cell cycle in gliomas⁴⁶, transfection of WTC EBdCas9 with p16 g1 resulted in 50% cell and colony reduction compared to no guide (−g) (FIG. 4E). Unlike gliomas that show an increase of G1/S phase⁴⁶, WTC p16 overexpression does not agree with this mechanism as downstream proteins are not present at this developmental stage⁴⁷. Instead, induction of EBdCas9 p16 g1 results in p16 overexpression and poor cell viability compared to no guide (−g) (FIG. 4F). Epigenetic tracing of EBdCas9 p16 g1 compared to NCdCas9 results in equivalent transcript expression of EB and NC, but 40 fold upregulation of p16 EBdCas9 cells compared to NC (FIG. 4G). Similarly, both EBdCas9 and NCdCas9 were recruited to p16 g1 region using mCherry™, however, only EBdCas9 showed reduction of H3K27me3 and EZH2 at targeted site using ChIPqPCR (FIG. 4G). To test whether p16 retains epigenetic memory as TBX18, we repeated the same experimental conditions (FIG. 4H) and learned that while p16 mRNA is present at 3D at 150 fold, 2 days later (5D), the transcript drops to 10 fold (Fig. I). This drop of mRNA expression may be due to very low cell count. Nevertheless, ChIPqPCR™ of p16 g1 site validated the recruitment of mCherry™ at 3D, but not at 5D, and the sustainability of reduced marks of H3K27me3 and EZH2 at day 3 as well as at day 5 (Fig. I). Accumulation of H3K4me3 marks at TSS of g1 compared to −g suggests active transcript up regulation using CUT and RUN⁴⁸ (FIG. 4J). As a control, H3K4me3 marks of neighboring p16 alternative splicing were unchanged for g1, sharpening the specificity of g1 p16 gene activation and elimination of off target affect. Since p16 is a challenging genomic area for adequate primer design, we were limited with neighborhood spreading analysis. However, p16 downstream locus (TSS) and upstream locus showed reduction of H3K27me3 (FIG. 4K). Using Element Navigation Tool™ validated the existence of TATAbox 38 bp of p16 g1, emphasizing the importance of EBdCas9/gRNA proximity for gene activation. We concluded that EBdCas9, but not NCdCas9 is able to activate P16 gene expression at precise loci and upregulate H3K4me3 epigenetic marks.

### EBdCas9 Directs Trophoblast Trans-Differentiation by Targeting CDX2 and GATA3

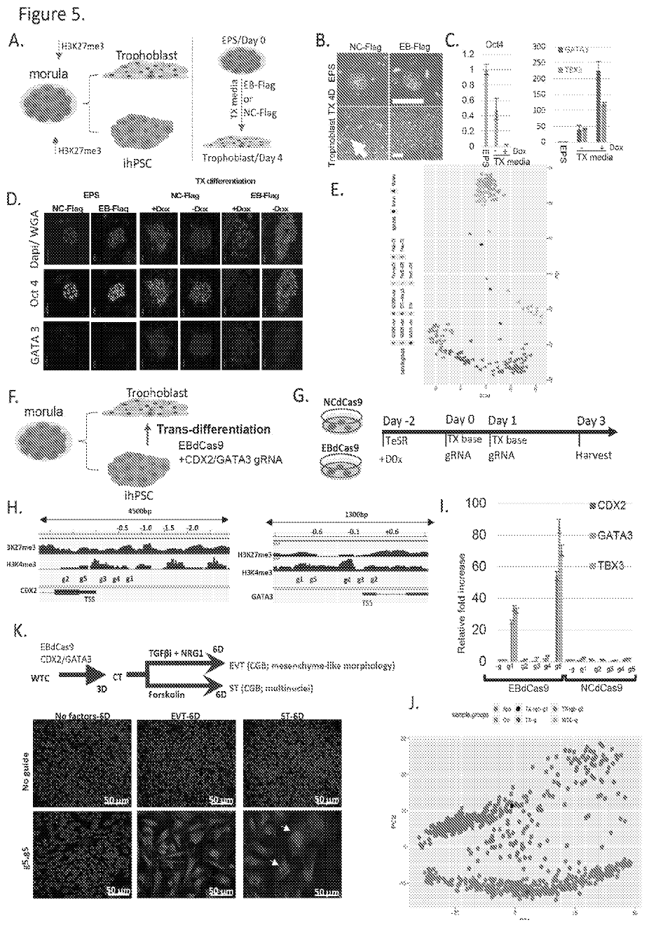

The first lineage bifurcation, trophoblast vs ICM cellular fate decision is dependent on PRC2³⁴. While overexpression of H3K27me3 is associated with ICM lineage, depletion of H3K27me3 marks is associated with trophoectoderm lineage^{34, 49-52}. As describe in our recent finding, expression of EB blocks the naïve to primed hESC transition, suggesting a role for H3K27 methylation³³. To test if inhibition of PRC2 activity in specific loci can change cell fate, we first asked whether the epigenetic biological inhibitor, EED binder (EB), is able to accelerate differentiation in well studied developmental transition. Recently 2 groups have generated culture condition that enabled the establishment of extended pluripotent stem cells (EPS) from either cleavage state of mouse embryos or human embryonic stem cells^{51, 52} The EPS cells stage a developmental potency and capability of making both embryonic, inner cell mass (ICM) and extraembryonic placental tissue, as trophoectoderm (TE) cell lineage^{51, 52}. Moreover, EPS epigenetic analysis validated bivalent gene enrichment of H3K27me3 and H3K4me3 in developmental processes^{51, 52} However, the functional mechanism that bifurcate the establishment of ICM and TE in isolated mouse rat and monkey preimplantation embryos was showed to be PRC2 dependent, coordinated via combinatorial regulation of EED and KDM6B³⁴. Specifically, repression of H3K27me3 at the chromatin domain of TE specific transcription factors CDX2 and GATA3 lead to their expression and results in TE lineage and repression of ICM lineage³⁴. Therefore, we determined the role of H3K27me3 marks in the transition of human EPS cells to TE first by using EB and later by targeting EBdCas9 to precise loci on key TE transcription factors. We first reprogrammed our previously generated WTC EB-Flag and WTC EBNC-Flag to EPS using LCDM reprogramming cocktail⁵². Once established we validated colony dome-shaped morphology, single cell colony efficiency and expression of pluripotency markers (data not shown). Once EPS EB-Flag and EPS EBNC-Flag have generated we set up an assay for TE differentiation to determine whether reduction of H3K27me3 marks using EB accelerates TE lineage choice (FIG. 5A). EPS EB-Flag and EPS EBNC-Flag were grown on MEF in LCDM media or Matrigel™ in TX media containing TGFb, FGF4 and heparin⁵³ and induced with Dox for 4d, the EB expressing cells differentiated faster and lost EPS colony morphology compared to no dox or the EBNC line (FIG. 5B). Relative mRNA expression also validated the accelerated reduction of Oct4 and the accelerated upregulation of TE markers GATA3 and TBX3 compared to no dox (FIG. 5C). Confocal imaging confirmed the tight, dome-shaped morphology, expression of nuclear stem cell transcription factor Oct4 and absence of Gata3 expression for both EPS EB-Flag and EPS EBNC-Flag (FIG. 5D). However, in the time course of 4 days TE differentiation and induction with dox, EB-Flag but not EBNC-Flag lost Oct4 marker expression and colony morphology compared to no dox or the EBNC line (FIG. 5D). Furthermore, during TE differentiation, EB-Flag abolished H3K27me3, EZH2 and Oct4, and upregulated CDX2, and GATA3 by whole cell protein analysis (data not shown). To investigate these fate changes in more detail we analyzed gene expression in these samples with RNA seq and utilized bioinformatics tools to identify the critical fate markers in these cells. Comparison of TE differentiated EPS EB-Flag with or without dox at 4 and 6 day time point as well as EPS EB-Flag differentiation to more mature ‘placental like’ cells Extravillous cyrotrophoblast (EVT)⁵⁵ to single cell transcriptome of early cynomolgus monkeys⁵⁶ resulted with advancement and acceleration of TE differentiation in EB expressing cells (FIG. 5E). The projection is based on 773 highly variable genes (standard deviation>2) in the monkey dataset. PC1 and PC2 correspond to developmental genes unbiased spread. TE differentiated EPS EB-Flag cells that were induced with dox during differentiation and expressed EB flag emigrate from post early or late epiblast (PostE-EPI; PostL-EPI), and shifted earlier towards post implantation partial trophectoderm (Post-paTE) and pre late trophectoderm (PreL-TE) compared to no dox EB-Flag TE differentiated cells. Also, the PCA clearly showed that all EB samples are far away from ICM and on the course of TE differentiation lineage. EVT EB-Flag cells were passaged for 3 times (in TSC conditioned media)⁵⁵ without dox and found to be closest to pre late trophectoderm (PreL-TE). This proved us that our TE differentiation is working and could continue with advancement of EB-Flag +dox longer than 6 day time point to accelerate TE differentiation into placental like cells. These results show that elimination of H3K27me3 marks by induction of the EB-Flag protein dramatically accelerates TE lineage differentiation.

Since global H3K27me3 reduction of EPS in TE differentiation resulted in accelerated post implantation partial trophectoderm like cells using EB-Flag, we tested if precise elimination of H3K27me3 marks in key transcription factors can also accelerated TE lineage cell choice.

During mouse blastocyst formation, the relative levels of EED and KDM6B, one of the histone demethylases, determine altered PRC2 complex recruitment and incorporation of H3K27me3 marks at the chromatin domains of target genes. Two trophectoderm (TE) lineage-specific transcription factors CDX2 and GATA3 show PRC2 dependent repression in the ICM³⁴. It remains to be seen if PRC2 activity is sufficient in the promoter regions of these two genes to distinguish the bifurcation between TE and ICM lineages. We challenged the question whether ICM like cells, such iPSC, which passed the bifurcation point, are able to transdifferentiate to TE using EBdCas9 targeting TE transcription factors CDX2 and GATA3 as gRNAs (FIG. 5F). WTC EBdCas9 cell lines were grown on Matrigel™ in TeSR (+Dox) for 2 days, and once gRNA transfection took place, the media was change to TX media base (+Dox) without factors (no TGFb, FGF4 and heparin) (FIG. 5G). This created a less biased differentiated environment for TE differentiation so EBdCas9/gRNAs are the sole drivers for transdifferentiation. CDX2 and GATA3 were tiled across the promoter and gene body area with 5 different guides (FIG. 5H). Since these two transcription factors are critical players in TE differentiation in mouse³⁴ we co-transfected g1 from CDX2 and g1 from GATA3 and applied it to all CDX2/GATA3 gRNA combination (g1/g1, g2/g2, . . . g5/g5). WTC EBdCas9 gRNA cocktail 1 and 5 resulted with gene activation between 20 to 80 fold not only CDX2 and GATA3 but also TE marker TBX3 compared to −g or NCdCas9 (FIG. 5I). gRNA cocktails 2-4 didn't show any gene activation to either CDX2 or GATA3 which may do with the proximity of the gRNA to TSS. Since g1 and g5 RNA resulted in outstanding CDX2 and GATA3 gene activation in WTC EBdCas9 cell lines, we decided to reprogram WTC EBdCas9 to EPS and measure gene activation prior to bifurcation point after gRNA plasmid transfection (data not shown). Unlike WTC EBdCas9 cell line, EPS EBdCas9 CDX2 and GATA3 gene activation increased between 100-500 fold, reminiscent of EB-Flag TE differentiation gene activation results (FIG. 5C). ChIPqPCR™ analysis of WTC EBdCas9 TE differentiation using g1/g1 and g5/g5 gRNA for CDX2 and GATA3 cocktail, resulted in mCherry recruitment and reduction of H3K27me3 and EZH2 at targeted genomic locus, compared to −g (data not shown). To investigate WTC-TE transdifferentiation changes in more detail we analyzed global transcriptomics of g1/g1 and g5/g5 RNA by RNA seq for CDX2 and GATA3 cocktail and compared them to trophoblast dataset as previously shown⁵⁷. PCA projection of developmental genes showed that WTC EBdCas9 with no gRNA transfection for either 3D TeSR or TX (base) conditions associated with control (WTC) dataset (FIG. 5J). More importantly, WTC EBdCas9 (TX) transfected with either g1/g1 or g5/g5 cocktail for CDX2 and GATA3 corresponded to TE differentiation, (FIG. 5J). Similarly, plotting these samples on single cell transcriptome of early cynomolgus monkeys⁵⁶ showed advanced emigration of g1/g1 or g5/g5 from postE-EPI/postL-EPI towards Post-paTE and PreL-TE. These data confirm that targeting EBdCas9 to eliminate H3K27me3 in precise CDX2 and GATA3 loci results in transdifferentiation of iPSC to TE. To prove WTC EBdCas9 g5 CDX2 and GATA3 cocktail is able to produce cytotrophoblast progenitor cells following 3D of trans-differentiation, we proceeded to specific extravillous cytotrophoblast (EVT) or Syncytiotrophoblast (ST) 6 days (6D) differentiation using TGFbi and Neuregulin or Forskolin, respectively. Immunofluorescence staining confirmed that 3D WTC EBdCas9 g5,g5 CDX2/GATA3 cocktail are able to differentiate to EVT and ST due to both positive staining of chorionic gonadotropin beta (CGB) and mesenchyme like and multinucleation morphology respectively⁵⁵. This also suggests that for the first time a novel design protein, a biological epigenetic remodeler, is able to change cell fate without artificial factors/inhibitors or manipulation at the DNA level.

### DISCUSSION

Control of epigenetic regulation holds new approach for treating human diseases free of traditional chemical drugs or DNA manipulation. Here we describe targeted inhibition of PRC2 that will, for the first time allow precise identification of functional H3K27me3 marks. This tool will also allow a study of the epigenetic memory of loss of specific H3K27 methyl marks. The technology reported here, which inhibits PRC2 function at specific genetic loci by utilizing an EB-dCas9 fusion and appropriate gRNA was fully able to: (1) target and inhibit PRC2 at a single nucleosome level, (2) reduce H3K27me3 at precise targeted locus, (3) induce targeted transcription, (4) mediate neighborhood spreading of remodeled epigenetic marks, (5) utilize epigenetic memory, (6) reveal licensing rules gene activation such as TATAbox region, (7) change cell functionality, and (8) transdifferentiate one cell fate to another.

Our elegant approach of targeted PRC2 inhibition allows organic expression of targeted gene, as the cell makes holistic decisions for transcript activation.

In summary, as a proof of concept, we tested the general applicability of EB-dCas9, by identifying the regions where gRNAs induce transcription in the following 5 bivalent genes: TBX18, p16, Klf4, Cdx2 and Gata3. In total, we have targeted 16 sites in enhancer and promoter regions upstream of five different genes, and observed significant transcriptional derepression in all genes, all together in 8 loci. In 7 of these, no effect was observed with the negative control NCdCas9; in the one case where NCdCas9 did have an effect the region targeted may be a repressor binding site. As NCdCas9 only differs from EB by two amino acid changes which completely abolish EED binding, taken together these results suggest that the guide RNA targeted EB-dCas9 fusions function as designed by locally inhibiting PRC2 activity. Finally, the combination of controlled epigenetic gain- and loss-of-function manipulations are the most desirable for elastic gene expression based epigenetic memory. Thus, the adaptive and efficient targeted PRC2 inhibition by EBdCas9 identifies functional H3K27me3 marks and mediates gene activation which can be harnessed both as a research epigenetic tool, in vivo biomedical research and as an approach for treating a wide range of human disease.

### Experimental Procedures

hiPSC and hESC Cell culture: The hiPSC line WTC #11, previously derived in the Conklin laboratory⁶², were cultured on Matrigel™ growth factor-reduced basement membrane matrix (Corning) in mTeSR media (StemCell Technologies). Naïve hESC [Elf-1(NIH hESC Registry #0156) had a normal, diploid karyotype⁶³. For 2iL-I-F conditions the cells were grown on a feeder layer of irradiated primary mouse embryonic fibroblasts in hESC media: DMEM/F-12 media supplemented with 20% knock-out serum replacer (KSR), 0.1 mM nonessential amino acids (NEAA), 1 mM sodium pyruvate, and penicillin/streptomycin (all from Invitrogen, Carlsbad, Calif.) and 0.1 mM β-mercaptoethanol (Sigma-Aldrich, St. Louis, Mo.). hESC media was supplemented with 1 μM GSK3 inhibitor (CHIR99021, Selleckchem), 1 μM of MEK inhibitor (PD0325901, Selleckchem), 10 ng/mL human LIF (Chemicon), 5 ng/mL IGF1 (Peprotech) and 10 ng/mL bFGF. For EPS conditions (extended pluripotency conditions)⁵² cells were grown in base medium containing 100 mL DMEM/F12, 100 mL Neurobasal, 1 mL N2 supplement, 2 mL B27 supplement, 1% GlutaMAX, 1% NEAA, 0.1 mM β-mercaptoethanol, penicillin-streptomycin and 5% KSR, and freshly supplemented with 10 ng/ml hLIF, GSK3i (1 μM), ROCKi′ (2 μM), (S)-(+)-Dimethindene maleate (2 μM; Tocris), Minocycline hydrochloride (2 μM; Santa Cruz Biotechnology) and IWR-endo-1 (0.5-1 μM; Selleckchem). Cells were adapted to EPS conditions for at least 3 passages before analysis. EPS cells were pushed toward differentiation using TX media⁵³: TX medium formulation was DMEM/F12 without HEPES and L-glutamine (Life Technologies), 64 mg/11-ascorbic acid-2-phosphate magnesium, 14 mg/l sodium selenite, 19.4 mg/l insulin, 543 mg/l NaHCO₃, 10.7 mg/l holo-transferrin (all Sigma-Aldrich), 25 ng/ml human recombinant FGF4 (Reliatech), 2 ng/ml human recombinant TGF-ß1 (PeproTech), 1 mg/ml heparin (Sigma-Aldrich), 2 mM L-glutamine, 1% penicillin, and streptomycin (all PAN-biotech). Medium was prepared without growth factors (TX-growth factors) and stored at 4° C. To prepare complete TX, the growth factors: FGF4, heparin, and TGF-b 1 were added prior to use. Medium was changed every other day. All cells were cultured at 37 degrees Celsius in 5% CO₂.

EBdCas9 and EBNCdCas9 plasmid construction: We used the AAVS1 TREG KRAB-dCas9 plasmid previously derived in the Conklin laboratory⁶² and preformed restriction digestion using PacI and AgeI. We ligated the EEDbinder-linker-dCas9-NLS-mCherry™ (EBdCas9) or EEDbinder Negative Control-linker-dCas9-NLS-mCherry™ (EBNCdCas9) to the cut plasmid, screened colonies and verified the sequence by Sanger sequencing.

EBdCas9 amino acid sequence
(SEQ ID NO: 58)
MINEIKKNAQERMDETVEQLKNELSKVRTGGGGTEERRLELAKQVVFAAN
RALIRVRTIALEAAWRLRMLGSDKEVNKRDISQALEEIEKLIKVAAKKIK
EVLEAKIKELREVMAVNSGGGGSRGGGSGGGGSGGGGSGGGGSGGGGMDK
KYSIGLAIGTNSVGWAVITDEYKVPSKKFKVLGNTDRHSIKKNLIGALLF
DSGETAEATRLKRTARRRYTRRKNRICYLQEIFSNEMAKVDDSFFHRLEE
SFLVEEDKKHERHPIFGNIVDEVAYHEKYPTIYHLRKKLVDSTDKADLRL
IYLALAHMIKFRGHFLIEGDLNPDNSDVDKLFIQLVQTYNQLFEENPINA
SGVDAKAILSARLSKSRRLENLIAQLPGEKKNGLFGNLIALSLGLTPNEK
SNFDLAEDAKLQLSKDTYDDDLDNLLAQIGDQYADLFLAAKNLSDAILLS
DILRVNTEITKAPLSASMIKRYDEHHQDLILLKALVRQQLPEKYKEIFFD
QSKNGYAGYIDGGASQEEFYKFIKPILEKMDGTEELLVKLNREDLLRKQR
TFDNGSIPHQIHLGELHAILRRQEDFYPFLKDNREKIEKILTFRIPYYVG
PLARGNSRFAWMTRKSEETITPWNFEEVVDKGASAQSFIERMTNFDKNLP
NEKVLPKHSLLYEYFTVYNELTKVKYVTEGMRKPAFLSGEQKKAIVDLLF
KTNRKVTVKQLKEDYFKKIECFDSVEISGVEDRFNASLGTYHDLLKIIKD
KDFLDNEENEDILEDIVLTLTLFEDREMIEERLKTYAHLFDDKVMKQLKR
RRYTGWGRLSRKLINGIRDKQSGKTILDFLKSDGFANRNFMQLIHDDSLT
FKEDIQKAQVSGQGDSLHEHIANLAGSPAIKKGILQTVKVVDELVKVMGR
HKPENIVIEMARENQTTQKGQKNSRERMKRIEEGIKELGSQILKEHPVEN
TQLQNEKLYLYYLQNGRDMYVDQELDINRLSDYDVDAIVPQSFLKDDSID
NKVLTRSDKNRGKSDNVPSEEVVKKMKNYWRQLLNAKLITQRKFDNLTKA
ERGGLSELDKAGFIKRQLVETRQITKHVAQILDSRMNTKYDENDKLIREV
KVITLKSKLVSDFRKDFQFYKVREINNYHHAHDAYLNAVVGTALIKKYPK
LESEFVYGDYKVYDVRKMIAKSEQEIGKATAKYFFYSNIMNFFKTEITLA
NGEIRKRPLIETNGETGEIVWDKGRDFATVRKVLSMPQVNIVKKTEVQTG
GFSKESILPKRNSDKLIARKKDWDPKKYGGFDSPTVAYSVLVVAKVEKGK
SKKLKSVKELLGITIMERSSFEKNPIDFLEAKGYKEVKKDLIIKLPKYSL
FELENGRKRMLASAGELQKGNELALPSKYVNFLYLASHYEKLKGSPEDNE
QKQLFVEQHKHYLDEIIEQISEFSKRVILADANLDKVLSAYNKHRDKPIR
EQAENIIHLFTLTNLGAPAAFKYFDTTIDRKRYTSTKEVLDATLIHQSIT
GLYETRIDLSQLGGDAYPYDVPDYASLGSGSPKKKRKVEDPKKKRKVDGI
GSGSNGSSGSATNFSLLKQAGDVEENPGPMVSKGEEDNMAIIKEFMRFKV
HMEGSVNGHEFEIEGEGEGRPYEGTQTAKLKVTKGGPLPFAWDILSPQFM
YGSKAYVKHPADIPDYLKLSFPEGFKWERVMNFEDGGVVTVTQDSSLQDG
EFIYKVKLRGTNFPSDGPVMQKKTMGWEASSERMYPEDGALKGEIKQRLK
LKDGGHYDAEVKTTYKAKKPVQLPGAYNVNIKLDITSHNEDYTIVEQYER
AEGRHSTGGMDELYK*
EBNCdCas9 amino acid sequence
(SEQ ID NO: 59)
MINEIKKNAQERMDETVEQLKNELSKVRTGGGGTEERRLELAKQVVEAAN
RALERVRTIALEAAWRLRMLGSDKEVNKRDISQALEEIEKLTKVAAKKIK
EVLEAKIKELREVMAVNSGGGGSRGGGSGGGGSGGGGSGGGGSGGGGMDK
KYSIGLAIGTNSVGWAVITDEYKVPSKKFKVLGNTDRHSIKKNLIGALLF
DSGETAEATRLKRTARRRYTRRKNRICYLQEIFSNEMAKVDDSFFHRLEE
SFLVEEDKKHERHPIFGNIVDEVAYHEKYPTIYHLRKKLVDSTDKADLRL
IYLALAHMIKERGHFLIEGDLNPDNSDVDKLFIQLVQTYNQLFEENPINA
SGVDAKAILSARLSKSRRLENLIAQLPGEKKNGLFGNLIALSLGLTPNFK
SNFDLAEDAKLQLSKDTYDDDLDNLLAQIGDQYADLFLAAKNLSDAILLS
DILRVNTEITKAPLSASMIKRYDEHHQDLTLLKALVRQQLPEKYKEIFFD
QSKNGYAGYIDGGASQEEFYKFIKPILEKMDGTEELLVKLNREDLLRKQR
TFDNGSIPHQIHLGELHAILRRQEDFYPFLKDNREKIEKILTFRIPYYVG
PLARGNSRFAWMTRKSEETITPWNFEEVVDKGASAQSFIERMTNFDKNLP
NEKVLPKHSLLYEYFTVYNELTKVKYVTEGMRKPAFLSGEQKKAIVDLLF
KTNRKVIVKQLKEDYFKKIECFDSVEISGVEDRFNASLGTYHDLLKIIKD
KDFLDNEENEDILEDIVLILTLFEDREMIEERLKTYAHLFDDKVMKQLKR
RRYTGWGRLSRKLINGIRDKQSGKTILDFLKSDGFANRNFMQLIHDDSLT
FKEDIQKAQVSGQGDSLHEHIANLAGSPAIKKGILQTVKVVDELVKVMGR
HKPENIVIEMARENQTTQKGQKNSRERMKRIEEGIKELGSQILKEHPVEN
TQLQNEKLYLYYLQNGRDMYVDQELDINRLSDYDVDAIVPQSFLKDDSID
NKVLIRSDKNRGKSDNVPSEEVVKKMKNYWRQLLNAKLITQRKFDNLTKA
ERGGLSELDKAGFIKRQLVETRQITKHVAQILDSRMNTKYDENDKLIREV
KVITLKSKLVSDFRKDFQFYKVREINNYHHAHDAYLNAVVGTALIKKYPK
LESEFVYGDYKVYDVRKMIAKSEQEIGKATAKYFFYSNIMNFFKTEITLA
NGEIRKRPLIETNGETGEIVWDKGRDFATVRKVLSMPQVNIVKKTEVQTG
GFSKESILPKRNSDKLIARKKDWDPKKYGGFDSPIVAYSVLVVAKVEKGK
SKKLKSVKELLGITIMERSSFEKNPIDFLEAKGYKEVKKDLIIKLPKYSL
FELENGRKRMLASAGELQKGNELALPSKYVNFLYLASHYEKLKGSPEDNE
QKQLFVEQHKHYLDEIIEQISEFSKRVILADANLDKVLSAYNKHRDKPIR
EQAENIIHLFTLINLGAPAAFKYFDTTIDRKRYTSTKEVLDATLIHQSIT
GLYETRIDLSQLGGDAYPYDVPDYASLGSGSPKKKRKVEDPKKKRKVDGI
GSGSNGSSGSATNFSLLKQAGDVEENPGPMVSKGEEDNMAIIKEFMRFKV
HMEGSVNGHEFEIEGEGEGRPYEGTQTAKLKVTKGGPLPFAWDILSPQFM
YGSKAYVKHPADIPDYLKLSFPEGFKWERVMNFEDGGVVTVTQDSSLQDG
EFIYKVKLRGTNFPSDGPVMQKKTMGWEASSERMYPEDGALKGEIKQRLK
LKDGGHYDAEVKTTYKAKKPVQLPGAYNVNIKLDITSHNEDYTIVEQYER
AEGRHSTGGMDELYK*

Insertion of inducible EBdCas9 and EBNCdCas9 into AAVS1 site of WTC and ElfI cells: 1×10⁶ cells of WTC p42 or Elf-1p17 were transfected with 5 μg AAVS1-TALEN R plasmid (Addgene #59026), 5 μg AAVS1-TALEN L plasmid (Addgene #59025), and 5 μg donor plasmid (AAVS1 TREG EBdCas9 or AAVS1 TREG EBNCdCas9) using the Amaxa Lonza Human stem cell Kit #2. The cells were then plated with 5 μM of Rock inhibitor (ROCKi) onto 10 cm with fresh media. Three days following the nucleofection, the cells were selected for neomycin resistance with Genetecin (50 μg/ml) for four days. 7 clones survived after selection and were expanded as a pool. Of these 14 clones, eight (CL #1,2,4,6,8,11,12,13) clones were plated onto Matrigel™ with or without doxycycline (2 μg/ml) and RNA was extracted in order to analyze the level of Cas9 expression by qPCR. Insertion of EBdCas9 or EBNCdCas9 into the AAVS1 site was confirmed by cellular genomic isolation, PCR amplification and Sanger sequencing.

### Guide RNA Design, Synthesis and Transfection

The gRNAs targeting TBX18, P16, KLF4, CDX2 and GATA3 genes were designed using the CRISPRscan™ web tools⁴¹ and ordered as T7-gRNA primers. A dsDNA fragment was synthesized from these primers by self-annealing PCR to a complementary scaffold primer (please make clearer), which is used to attach the guide to dCas9. The dsDNA fragment was followed by Q5 High Fidelity-based PCR (New England Biolabs). This 120 bp strand served as template for IVT (MAXIscript T7 kit, applied Biosystems). The RNA was then purified using Pellet Paint® Co-Precipitant (Novagen). WTC EBdCas9 or EBNCdCas9cells were seeded at day 0, and treated with doxycycline (2 μg/ml) for 2 days before and during transfection. On day 2 cells were transfected with gRNAs using Lipofectamine RNAiMAX™ (Life Technologies). gRNA was added at a 40 nM final concentration when added alone or 20 nM in co-gRNA transfection. A second transfection was performed after 24 h. Two days after the last gRNA transfection, cells were harvest for either DNA, RNA and protein, ChIPqPCR™, or Cut and Run analysis.

CRISPR Off-Target: The potential off targets of the gRNA were identified using Crispr-RGEN™'s Cas-OFFinder™ tool⁶⁴. The top predicted off targets were then amplified by GoTaq™ PCR and sequenced.

### DNA Extraction and Sequencing

Genomic DNA was collected using DNAzol™ reagent (Invitrogen) according to manufacturer's instructions and quantified using Nanodrop™ ND-1000. Genomic regions flanking the AAVS1 were PCR amplified with the designed primers, purified by PCR Purification Kit (Invitrogen) and sent to Genewiz™ for sequencing.

### RNA Extraction and RT-qPCR Analysis

RNA was extracted using Trizol™ (Life Technologies) according to manufacturer's instructions. RNA samples were treated with Turbo DNase (ThermoFischer) and quantified using Nanodrop™ ND-1000. Reverse transcription was performed using iScript™ (BioRad). 10 ng of cDNA was used to perform qRT-PCR using SYBR™ Green, with suitable primers on an Applied Biosystems 7300 real time PCR system with PCR conditions as stage 1 50° C. for 2 mins, stage 2 as 95° C. for 10 mins, 95° C. for 15 sec, 60° C. for 1 min(40 Cycles). ß-actin was used as an endogenous control.

### Protein Extraction and Western Blot Analysis

Cells were lysed directly on the plate with lysis buffer containing 20 mM Tris-HCl pH 7.5, 150 mM NaCl, 15% Glycerol, 1% Triton x-100, 1M [symbol] -Glycerolphosphate, 0.5M NaF, 0.1M Sodium Pyrophosphate, Orthovanadate, PMSF and 2% (or 10%?) SDS. 25 U of Benzonase® Nuclease (EMD Chemicals, Gibbstown, N.J.) was added to the lysis buffer right before use. Proteins were quantified by Bradford assay (Bio-rad), using BSA (Bovine Serum Albumin) as Standard using the EnWallac™ Vision. The protein samples were combined with the 4× Laemli sample buffer (900 μl of sample buffer and 100 μl β-Mercaptoethanol), heated (95° C., 5 mins) and run on SDS-PAGE (protean TGX pre-casted gradient gel, 4%-20%, Bio-rad) and transferred to the Nitro-Cellulose membrane (Bio-Rad) by semi-dry transfer (Bio-Rad). Membrane was blocked for 1 hr with 5% milk, and incubated in the primary antibodies overnight in 4° C. The antibodies used for western blot were β-Tubulin III (Promega G7121, 1:1000), Cas9 (Cell Signaling 1:1000), Oct-4 (Santa Cruz sc-5279, 1:1000, Novus Biologicals NB110-90606, 1:500), H3K27me3 (Active Motive 39155 1:1000), EZH2 (Cell Signaling D2C9, 1:1000). (CGB 1:200 cell signaling) The membranes were then incubated with secondary antibodies (1:10000, goat anti-rabbit or goat anti-mouse IgG HRP conjugate (Bio-Rad) for 1 hr and the detection was performed using the immobilon-luminol reagent assay (EMP Millipore).

### Immunostaining and Confocal Imaging

Cells were fixed in 4% paraformaldehyde in PBS for 15 min, permeabilized for 10 min in 0.1% Triton X-100 and blocked for 1h in 2% BSA. The cells were then incubated in primary antibody overnight, washed with PBS (3×5 min), incubated with the secondary antibody in 2% BSA for 1 hr, washed (4×10 mins, adding 1 μg/ml DAPI in 2nd wash), mounted (2% of n-Propyl Gallate in 90% Glycerol and 10% PBS) and stored in the 4° C. Analysis was done on a Leica TCS-SPE Confocal microscope using a 40× objective and Leica Software. The antibodies for immunostaining were anti-GATA3 (cell signaling, 1:200), anti-Oct-4 (Novus Biologicals, 1:150), anti p16 (Santa Cruz 1:200) and Alexa 488- or Alexa 647-conjugated secondary antibodies (Molecular Probes).

### ChIP-qPCR Analysis

Matrix ChIP™ was performed on WTC EBdCas9 samples transfected with or without KLF4 gRNA utilizing a previously published microplate-based chromatin immunoprecipitation method (Matrix ChIP™)⁶⁵. Briefly, 96-well microplates with reactin-bind protein A (Pierce) were incubated with protein A on a low-speed shaker at room temperature overnight. The next day, the wells were blocked with blocking buffer containing 5% BSA and immunoprecipitation buffer on a shaker at 40° C. for 60 min. Simultaneously, chromatin samples (see sequential ChIP™ to obtain chromatin) with blocking buffer and antibody were added to a new UV-modified polypropylene 96-well microplates (Genemate) and incubated in ultrasonic bath for 60 min at 4° C. The blocking buffer was aspirated from the protein A-coated plate, and the chromatin+antibody mix was added to the wells and incubated in the ultrasonic bath for 60 min at 4° C. The chromatin samples were washed 3 times with immunoprecipitation buffer and then TE buffer. Finally, elution buffer containing 25 mM Tris base, 1 mM EDTA (pH10) with proteinase K 200 μg/ml was added to the wells, then shaken for 30 s at 1400 rpms and incubated for 45 min at 55° C. and then 10 min at 95° C. The 96-well plates were then briefly agitated and centrifuged for 3 min at −500 g at 4° C. and were used for PCR. The antibodies utilized for Matrix ChIP™ were H3K27me3 (Active motif), H3K27ac (Active motif), EZH2 (cell signaling). Matrix ChIP experiments were performed in triplicate followed by qPCR in 6-12 replicates.

### Cut and Run Analysis

1 million WTC EBdCas9 cells gRNA transfected or not were harvested by centrifugation (600 g, 3 min in a swinging bucket rotor) and washed in ice cold phosphate-buffered saline (PBS). Nuclei were isolated by hypotonic lysis in 1 ml NE1 (20 mM HEPES-KOH pH 7.9; 10 mM KCl; 1 mM MgCl₂; 0.1% Triton X-100; 20% Glycerol) for 5 min on ice followed by centrifugation as above. Nuclei were briefly washed in 1.5 ml Buffer 1 (20 mM HEPES pH 7.5; 150 mM NaCl; 2 mM EDTA; 0.5 mM Spermidine; 0.1% BSA) and then washed in 1.5 ml Buffer 2 (20 mM HEPES pH 7.5; 150 mM NaCl; 0.5 mM Spermidine; 0.1% BSA). Nuclei were resuspended in 500 μl Buffer 2 and 10 μl antibody was added and incubated at 4° C. for 2 hr. Nuclei were washed 3× in 1 ml Buffer 2 to remove unbound antibody. Nuclei were resuspended in 300 μl Buffer 2 and 5 μl pA-MN added and incubated at 4° C. for 1 hr. Nuclei were washed 3× in 0.5 ml Buffer 2 to remove unbound pA-MN. Tubes were placed in a metal block in ice-water and quickly mixed with 100 mM CaCl₂ to a final concentration of 2 mM. The reaction was quenched by the addition of EDTA and EGTA to a final concentration of 10 mM and 20 mM respectively and 1 ng of mononucleosome-sized DNA fragments from Drosophila DNA added as a spike-in. Cleaved fragments were liberated into the supernatant by incubating the nuclei at 4° C. for 1 hr, and nuclei were pelleted by centrifugation as above. DNA fragments were extracted from the supernatant and used for the construction of sequencing libraries. We have also adapted this protocol for use with magnetic beads⁴⁸.

### RNA-Seq Data Analysis

RNA-seq samples were aligned to hg19 using Tophat™ [31](version 2.0.13). Gene-level read counts were quantified using htseq-count using Ensembl™ GRCh37 gene annotations. Processed single cell RNA-seq data from Nakamura et al⁵⁶ were used. Only genes expressed above 10 Reads Per Million in 3 or more samples were kept. t-SNE was performed with the Rtsne package, using genes with the top 20% variance across samples. Cluster labels from Nakamura et al were used. A Principle Component Analysis (PCA) was performed using all of the cynomolgus monkey samples from Nakamura et al⁵⁶ using R software. Genes used in the analysis were restricted to defined homologs expressed at non-zero Transcripts Per Million (TPM) in human in vitro cell lines, and in the preprocessed mouse and cynomolgus monkey single cell samples from Nakamura et al. RNA-seq data from human cell lines were corrected for batch effects using ComBat™⁶⁶. Human bulk RNA-seq samples were projected onto the PCA coordinate via matrix multiplication. Human, cynomolgus monkey and mouse RNA-seq data were separately centered and scaled within each species before PCA and projection was performed.

### REFERENCES

1. Margueron, R. & Reinberg, D. The Polycomb complex PRC2 and its mark in life. Nature 469, 343-349 (2011).
2. Lee, C. H. et al. Automethylation of PRC2 promotes H3K27 methylation and is impaired in H3K27M pediatric glioma. Genes Dev 33, 1428-1440 (2019).
3. Kasinath, V., Poepsel, S. & Nogales, E. Recent Structural Insights into Polycomb Repressive Complex 2 Regulation and Substrate Binding. Biochemistry 58, 346-354 (2019).
4. Laugesen, A., Hojfeldt, J. W. & Helin, K. Role of the Polycomb Repressive Complex 2 (PRC2) in Transcriptional Regulation and Cancer. Cold Spring Harb Perspect Med 6 (2016).
5. Coleman, R. T. & Struhl, G. Causal role for inheritance of H3K27me3 in maintaining the OFF state of a Drosophila HOX gene. Science 356 (2017).
6. Laprell, F., Finkl, K. & Muller, J. Propagation of Polycomb-repressed chromatin requires sequence-specific recruitment to DNA. Science 356, 85-88 (2017).
7. Yu, J. R., Lee, C. H., Oksuz, O., Stafford, J. M. & Reinberg, D. PRC2 is high maintenance. Genes Dev 33, 903-935 (2019).
8. Lee, C. H. et al. Allosteric Activation Dictates PRC2 Activity Independent of Its Recruitment to Chromatin. Mol Cell 70, 422-434 e426 (2018).
9. Cooper, S. et al. Jarid2 binds mono-ubiquitylated H2A lysine 119 to mediate crosstalk between Polycomb complexes PRC1 and PRC2. Nat Commun 7, 13661 (2016).
10. Brockdorff, N. Polycomb complexes in X chromosome inactivation. Philos Trans R Soc Lond B Biol Sci 372 (2017).
11. Holoch, D. & Margueron, R. Mechanisms Regulating PRC2 Recruitment and Enzymatic Activity. Trends Biochem Sci 42, 531-542 (2017).
12. Francis, N.J., Follmer, N. E., Simon, M. D., Aghia, G. & Butler, J. D. Polycomb proteins remain bound to chromatin and DNA during DNA replication in vitro. Cell 137, 110-122 (2009).
13. Eskeland, R. et al. Ring1B compacts chromatin structure and represses gene expression independent of histone ubiquitination. Mol Cell 38, 452-464 (2010).
14. Illingworth, R. S. et al. The E3 ubiquitin ligase activity of RING1B is not essential for early mouse development. Genes Dev 29, 1897-1902 (2015).
15. Pengelly, A. R., Kalb, R., Finkl, K. & Muller, J. Transcriptional repression by PRC1 in the absence of H2A monoubiquitylation. Genes Dev 29, 1487-1492 (2015).
16. Oksuz, O. et al. Capturing the Onset of PRC2-Mediated Repressive Domain Formation. Mol Cell 70, 1149-1162 e1145 (2018).
17. Hawkins, R. D. et al. Distinct epigenomic landscapes of pluripotent and lineage-committed human cells. Cell Stem Cell 6, 479-491 (2010).
18. Battle, S. L. et al. Enhancer Chromatin and 3D Genome Architecture Changes from Naive to Primed Human Embryonic Stem Cell States. Stem Cell Reports 12, 1129-1144 (2019).
19. Pengue, G. & Lania, L. Kruppel-associated box-mediated repression of RNA polymerase II promoters is influenced by the arrangement of basal promoter elements. Proc Nall Acad Sci USA 93, 1015-1020 (1996).
20. Groner, A. C. et al. KRAB-zinc finger proteins and KAP1 can mediate long-range transcriptional repression through heterochromatin spreading. PLoS Genet 6, e1000869 (2010).
21. Gao, R. et al. Depletion of histone demethylase KDM2A inhibited cell proliferation of stem cells from apical papilla by de-repression of p15INK4B and p27Kip 1. Mol Cell Biochem 379, 115-122 (2013).
22. Kearns, N. A. et al. Functional annotation of native enhancers with a Cas9-histone demethylase fusion. Nat Methods 12, 401-403 (2015).
23. Shechner, D. M., Hacisuleyman, E., Younger, S. T. & Rinn, J. L. Multiplexable, locus-specific targeting of long RNAs with CRISPR-Display. Nat Methods 12, 664-670 (2015).
24. Thakore, P. I. et al. Highly specific epigenome editing by CRISPR-Cas9 repressors for silencing of distal regulatory elements. Nat Methods 12, 1143-1149 (2015).
25. Amabile, A. et al. Inheritable Silencing of Endogenous Genes by Hit-and-Run Targeted Epigenetic Editing. Cell 167, 219-232 e214 (2016).
26. Pradeepa, M. M. et al. Histone H3 globular domain acetylation identifies a new class of enhancers. Nat Genet 48, 681-686 (2016).
27. Chavez, A. et al. Comparison of Cas9 activators in multiple species. Nat Methods 13, 563-567 (2016).
28. Gilbert, L. A. et al. Genome-Scale CRISPR-Mediated Control of Gene Repression and Activation. Cell 159, 647-661 (2014).
29. Adamo, A. et al. LSD1 regulates the balance between self-renewal and differentiation in human embryonic stem cells. Nat Cell Biol 13, 652-659 (2011).
30. Goodman, R. H. & Smolik, S. CBP/p300 in cell growth, transformation, and development. Genes Dev 14, 1553-1577 (2000).
31. O'Geen, H. et al. dCas9-based epigenome editing suggests acquisition of histone methylation is not sufficient for target gene repression. Nucleic Acids Res 45, 9901-9916 (2017).
32. Fang, D. et al. H3K27me3-mediated silencing of Wilms Tumor 1 supports the proliferation of brain tumor cells harboring the H3.3K27M mutation. bioRxiv (2017).
33. Moody, J. D. et al. First critical repressive H3K27me3 marks in embryonic stem cells identified using designed protein inhibitor. Proc Natl Acad Sci USA 114, 10125-10130 (2017).
34. Saha, B. et al. EED and KDM6B coordinate the first mammalian cell lineage commitment to ensure embryo implantation. Mol Cell Biol 33, 2691-2705 (2013).
35. Kim, W. et al. Targeted disruption of the EZH2-EED complex inhibits EZH2-dependent cancer. Nat Chem Biol 9, 643-650 (2013).
36. Kong, X. et al. Astemizole arrests the proliferation of cancer cells by disrupting the EZH2-EED interaction of polycomb repressive complex 2. J Med Chem 57, 9512-9521 (2014).
37. Knutson, S. K. et al. Durable tumor regression in genetically altered malignant rhabdoid tumors by inhibition of methyltransferase EZH2. Proc Natl Acad Sci USA 110, 7922-7927 (2013).
38. Mandegar, M. A. et al. CRISPR Interference Efficiently Induces Specific and Reversible Gene Silencing in Human iPSCs. Cell Stem Cell 18, 541-553 (2016).
39. Wiese, C. et al. Formation of the sinus node head and differentiation of sinus node myocardium are independently regulated by Tbx18 and Tbx3. Circ Res 104, 388-397 (2009).
40. Kapoor, N., Liang, W., Marban, E. & Cho, H. C. Direct conversion of quiescent cardiomyocytes to pacemaker cells by expression of Tbx18. Nat Biotechnol 31, 54-62 (2013).
41. Moreno-Mateos, M. A. et al. CRISPRscan: designing highly efficient sgRNAs for CRISPR-Cas9 targeting in vivo. Nat Methods 12, 982-988 (2015).
42. Bomsztyk, K. et al. PIXUL-ChIP: integrated high-throughput sample preparation and analytical platform for epigenetic studies. Nucleic Acids Res 47, e69 (2019).
43. Sloutskin, A. et al. ElemeNT: a computational tool for detecting core promoter elements. Transcription 6, 41-50 (2015).
44. Piunti, A. et al. Therapeutic targeting of polycomb and BET bromodomain proteins in diffuse intrinsic pontine gliomas. Nat Med 23, 493-500 (2017).
45. Mohammad, F. et al. EZH2 is a potential therapeutic target for H3K27M-mutant pediatric gliomas. Nat Med 23, 483-492 (2017).
46. Cordero, F. J. et al. Histone H3.3K27M Represses p16 to Accelerate Gliomagenesis in a Murine Model of DIPG. Mol Cancer Res 15, 1243-1254 (2017).
47. Itahana, Y. et al. Histone modifications and p53 binding poise the p21 promoter for activation in human embryonic stem cells. Sci Rep 6, 28112 (2016).
48. Skene, P. J. & Henikoff, S. An efficient targeted nuclease strategy for high-resolution mapping of DNA binding sites. Elife 6 (2017).
49. Banaszynski, L. A. et al. Hira-dependent histone H3.3 deposition facilitates PRC2 recruitment at developmental loci in ES cells. Cell 155, 107-120 (2013).
50. Liu, X. et al. Distinct features of H3K4me3 and H3K27me3 chromatin domains in pre-implantation embryos. Nature 537, 558-562 (2016).
51. Yang, J. et al. Establishment of mouse expanded potential stem cells. Nature 550, 393-397 (2017).
52. Yang, Y. et al. Derivation of Pluripotent Stem Cells with In Vivo Embryonic and Extraembryonic Potency. Cell 169, 243-257 e225 (2017).
53. Kubaczka, C. et al. Derivation and maintenance of murine trophoblast stem cells under defined conditions. Stem Cell Reports 2, 232-242 (2014).
54. Sperber, H. et al. The metabolome regulates the epigenetic landscape during naive-to-primed human embryonic stem cell transition. Nat Cell Biol 17, 1523-1535 (2015).
55. Okae, H. et al. Derivation of Human Trophoblast Stem Cells. Cell Stem Cell 22, 50-63 e56 (2018).
56. Nakamura, T. et al. Single-cell transcriptome of early embryos and cultured embryonic stem cells of cynomolgus monkeys. Sci Data 4, 170067 (2017).
57. Krendl, C. et al. GATA2/3-TFAP2A/C transcription factor network couples human pluripotent stem cell differentiation to trophectoderm with repression of pluripotency. Proc Natl Acad Sci USA 114, E9579-E9588 (2017).
58. Konermann, S. et al. Genome-scale transcriptional activation by an engineered CRISPR-Cas9 complex. Nature 517, 583-588 (2015).
59. Liao, H. K. et al. In Vivo Target Gene Activation via CRISPR/Cas9-Mediated Trans-epigenetic Modulation. Cell 171, 1495-1507 e1415 (2017).
60. Joung, J. et al. Genome-scale CRISPR-Cas9 knockout and transcriptional activation screening. Nat Protoc 12, 828-863 (2017).
61. Weltner, J. et al. Human pluripotent reprogramming with CRISPR activators. Nat Commun 9, 2643 (2018).
62. Kreitzer, F. R. et al. A robust method to derive functional neural crest cells from human pluripotent stem cells. Am J Stem Cells 2, 119-131 (2013).
63. Ware, C. B. et al. Derivation of naive human embryonic stem cells. Proc Natl Acad Sci USA 111, 4484-4489 (2014).
64. Bae, S., Park, J. & Kim, J. S. Cas-OFFinder: a fast and versatile algorithm that searches for potential off-target sites of Cas9 RNA-guided endonucleases. Bioinformatics 30, 1473-1475 (2014).
65. Flanagin, S., Nelson, J. D., Castner, D. G., Denisenko, O. & Bomsztyk, K. Microplate-based chromatin immunoprecipitation method, Matrix ChIP: a platform to study signaling of complex genomic events. Nucleic Acids Res 36, e17 (2008).
66. Johnson, W. E., Li, C. & Rabinovic, A. Adjusting batch effects in microarray expression data using empirical Bayes methods. Biostatistics 8, 118-127 (2007).

We claim:

1. A composition, comprising:
(a) an embryonic ectoderm development (EED) polypeptide binder (EB) domain; and
(b) a CRISPR associated protein 9 (CAS9) domain linked to the EB domain.

2. The composition of claim 1, wherein the EB domain and the CAS9 domain are expressed in a fusion protein, and may be separated by an amino acid linker connecting the EB domain and the CAS domain.

3. The composition of claim 1 or 2, wherein the EB domain comprises the motif F(X1)ANR(X2)(X3)I (SEQ ID NO:60), wherein X1, X2, and X3 are any amino acid.

4. The composition of claim 3, wherein X1 is a hydrophobic amino acid, including but not limited to V, A, or I.

5. The composition of claim 3 or 4, wherein at least one of X2 and X3 is a polar amino acid, including but not limited to L or K.

6. The composition of any one of claims 1-5, wherein the EB domain comprises the amino acid sequence at least 50%, 55%, 60%, 65%, 70%, 75%, 80%, 85%, 90%, 91%, 92%, 93%, 94%, 95%, 96%, 97%, 98%, or 100% identical along to the amino acid sequence of any one of SEQ ID NOS:1-9, 11, and 13.

7. The composition of any one of claims 1-6, wherein the EB domain comprises the amino acid sequence at least 50%, 55%, 60%, 65%, 70%, 75%, 80%, 85%, 90%, 91%, 92%, 93%, 94%, 95%, 96%, 97%, 98%, or 100% identical along the length of SEQ ID NO:13, wherein the highlighted residues are not modified.

8. The composition of any one of claims 2-7, wherein the amino acid linker comprises a sequence that may include, but is not limited to, a sequence having the amino acid sequence selected from the group consisting of SEQ ID NO:14-33.

9. The composition of any one of claims 2-8, wherein the amino acid linker comprises the amino acid sequence of SEQ ID NO:33.

10. The composition of any one of claims 1-9, wherein the Cas9 domain comprises the amino acid sequence at least 50%, 55%, 60%, 65%, 70%, 75%, 80%, 85%, 90%, 91%, 92%, 93%, 94%, 95%, 96%, 97%, 98%, or 100% identical to the amino acid sequence of any one of SEQ ID NO:34 or SEQ ID NO:40-57.

11. The composition of any one of claims 1-10, wherein the Cas9 domain comprises the amino acid sequence at least 50%, 55%, 60%, 65%, 70%, 75%, 80%, 85%, 90%, 91%, 92%, 93%, 94%, 95%, 96%, 97%, 98%, or 100% identical to the amino acid sequence of SEQ ID NO:34.

12. The composition of any one of claims 1-11 further comprising a localization domain.

13. The composition of claim 12, wherein the localization domain comprises the amino acid sequence at least 50%, 55%, 60%, 65%, 70%, 75%, 80%, 85%, 90%, 91%, 92%, 93%, 94%, 95%, 96%, 97%, 98%, or 100% identical to the amino acid sequence of SEQ ID NO:35.

14. The composition of any one of claims 1-13, further comprising a detectable domain.

15. The composition of claim 14, wherein the detectable domain comprises the amino acid sequence at least 50%, 55%, 60%, 65%, 70%, 75%, 80%, 85%, 90%, 91%, 92%, 93%, 94%, 95%, 96%, 97%, 98%, or 100% identical to the amino acid sequence of SEQ ID NO:36.

16. The composition of any one of claims 2-15, wherein the polypeptide comprises the amino acid sequence at least 50%, 55%, 60%, 65%, 70%, 75%, 80%, 85%, 90%, 91%, 92%, 93%, 94%, 95%, 96%, 97%, 98%, or 100% identical to the amino acid sequence of SEQ ID NO:37.

17. The composition of any one of claims 2-15, wherein the polypeptide comprises the amino acid sequence at least 50%, 55%, 60%, 65%, 70%, 75%, 80%, 85%, 90%, 91%, 92%, 93%, 94%, 95%, 96%, 97%, 98%, or 100% identical to the amino acid sequence of SEQ ID NO:38.

18. The composition of any one of claims 1-17, bound to a scaffold, including but not limited to a nanoparticle, virus-like particle, or other polypeptide scaffold.

19. A nucleic acid encoding the polypeptide of any one of claims 2-18.

20. An expression vector comprising the nucleic acid of claim 19 operatively linked to a suitable control sequence.

21. A host cell comprising the nucleic acid of claim 19 or the expression vector of claim 20.

22. The host cell of claim 21, wherein the host cell is a stable host cell capable of expressing the polypeptide.

23. The host cell of claim 20 or 21, further comprising one or more guide RNAs (gRNA) selective for one or more particular genes, a nucleic acid encoding the one or more guide RNAs, and/or an expression vector comprising a nucleic acid encoding the one or more guide RNAs operatively linked to a suitable control sequence.

24. The host cell of claim 23, wherein the host cell comprises an expression vector comprising a nucleic acid encoding the one or more guide RNAs operatively linked to a suitable control sequence, wherein the control sequence comprises a TATA box within 50-100 base pairs of the nucleic acid encoding the one or more guide RNAs.

25. A pharmaceutical composition comprising the composition, nucleic acid, expression vector, and/or host cell of any previous claim and a pharmaceutically acceptable carrier.

26. The pharmaceutical composition of claim 25, further comprising a nucleic acid encoding the one or more guide RNAs operatively linked to a suitable control sequence, wherein the control sequence comprises a TATA box within 50-100 base pairs of the nucleic acid encoding the one or more guide RNAs.

27. A kit comprising:
(a) an active composition of any one of claims 1-18, nucleic acid of claim 19, expression vector of claim 20, host cell of claim 21-24, and/or pharmaceutical composition of claim 25-26; and
(b) a control composition, nucleic acid, expression vector, host cell, and/or pharmaceutical composition that is identical to the active composition, the active nucleic acid, the active expression vector, the active host cell, and/or the pharmaceutical composition, except that the EB domain is inactive (i.e.: does not bind to EED), and/or the control nucleic acid encodes an inactive EB domain.

28. The kit of claim 27, wherein the inactive EB domain comprises the amino acid sequence at least 50%, 55%, 60%, 65%, 70%, 75%, 80%, 85%, 90%, 91%, 92%, 93%, 94%, 95%, 96%, 97%, 98%, or 100% identical to the amino acid sequence of SEQ ID NO:13, wherein the highlighted residues are modified to polar or charged amino acids.
(SEQ ID NO: 13)
MINEIKKNAQERMDETVEQLKNELSKVRTGGGGTEERRLELAKQVVFAAN
RALIRVRTIALEAAWRLRMLGSDKEVNKRDISQALEEIEKLTKVAAKKIK
EVLEAKIKELREVMAVN

29. The kit of claim 27 or 28, wherein the inactive EB domain comprises the amino acid sequence at least 50%, 55%, 60%, 65%, 70%, 75%, 80%, 85%, 90%, 91%, 92%, 93%, 94%, 95%, 96%, 97%, 98%, or 100% identical to the amino acid sequence of SEQ ID NO:10, 12, or 39, wherein the highlighted residues are not modified
>EB15.2NC
(SEQ ID NO: 10)
HMGQRWELALQRFWDYLRWVQTLSEQVQEELLSDKAIEELAALAKET
ERELRNYIAELSKQLTPVAEETKRQLATTLVEVANRLKETMRTIMLE
LLRYRIAVNALNGQSTEDLRRNLAENLRKSRDDLLITADKLQRVLAV
YQAGALE
>EB22.2NC
(SEQ ID NO: 12)
HMINEIKKNAQERMDETVEQLKNELSKVRTGGGGTEERRLELAKQVV
EAANRALERVRTIALEAAWRLRMLGSDKEVNKRDISQALEEIEKLTK
VAAKKIKEVLEAKIKELREVLE
(SEQ ID NO: 39)
MINEIKKNAQERMDETVEQLKNELSKVRTGGGGTEERRLELAKQVVE
AANRALERVRTIALEAAWRLRMLGSDKEVNKRDISQALEEIEKLTKV
AAKKIKEVLEAKIKELREVMAVN

30. A method for use of the composition, the nucleic acid, the expression vector, the host cell, the pharmaceutical composition, and/or the kit of any preceding claim for gene activation in a biological cell.

31. The method of claim 30, comprising:
(a) providing the host cell of any one of claims 21-24, which comprises the expression vector of claim 20 and/or the nucleic acid of claim 19;
(b) contacting the host cell with a guide RNA (gRNA) selective for a gene to be activated, including but not limited to adding the gRNA at the time of gene activation, or providing host cells that express the gRNA (including but not limited to host cells transfected with a viral construct or transiently or stably transfected with a plasmid, in each case having an appropriate promoter (including but not limited to u6) controlling gRNA expression); and
(c) culturing the cells under conditions suitable to promote expression of the polypeptide in the host cell, wherein the polypeptide directs PRC2 disruption at the gene targeted by the gRNA, thus activating the gene.

32. The method of claim 30, comprising:
(a) providing a host cell comprising a composition of any one of claims 1-18 and one or more guide RNA (gRNA) selective for a gene(s) to be activated; and
(b) culturing the cells under conditions suitable to promote targeting of the gene(s) to be activated with the gRNA, wherein the composition directs PRC2 disruption at the gene targeted by the gRNA, thus activating the gene.

33. The method of any one of claims 30-32, wherein the biological cell is present within a subject having glioblastoma, wherein the gene targeted by the gRNA comprises the p16 gene, and wherein the gene activation serves to treat the glioblastoma.

34. The method of any one of claims 31-33, wherein the one or more gRNA is encoded by a nucleic acid operatively linked to a suitable control sequence, wherein the control sequence comprises a TATA box within 50-100 base pairs of the nucleic acid encoding the gRNA.


SEQUENCE LISTING (63 sequences)

SEQ ID NO: 1 - protein, 148 aa, Artificial Sequence
  Synthetic peptide
HMGQRWELALQRFWDYLRWVQTLSEQVQEELLSDKAIEELAALAKETERELRNYIAELSKQLTPVAEETKRQLATTLVFVANRLKITMRTIMLELLWYRIAVNALNGQSTEDLRRNLAENLRKSRDDLLITADKLQRVLAVYQAGALE

SEQ ID NO: 2 - protein, 148 aa, Artificial Sequence
  Synthetic peptide
HMGQRWELALQRFWDYLRWVQTLSEQVQEELLTKQVTRELSELRSNTLRELAAYKSELEEQLTPVAEETRARLSKELATTAKALLFVMNRILIALRTYILAVLWMDGTSTEKLRVQLASDLRQLRDKLLRAADELQKVLAVYQAGALE

SEQ ID NO: 3 - protein, 113 aa, Artificial Sequence
  Synthetic peptide
HMGGWRREYPPITSDQQRQEYKRNFDTGLREAARLVFILNRIRIQLRTLILELIWADEESRRYKQAADEYNRLKQVKGSADYKSKRDIVLELAKKLEHIAKMVKDYDRQKTLE

SEQ ID NO: 4 - protein, 116 aa, Artificial Sequence
  Synthetic peptide
HMIREALKDAQEKMKKAVQVAEDDLSTIRTGGGGTQERRKELVDQAIHKGKEAEQSVKKIMEEAQKELRRIRKEGEAGEDEVGKASAMLTFITNRYKITIRTLVLEKMWRLLAVLE

SEQ ID NO: 5 - protein, 113 aa, Artificial Sequence
  Synthetic peptide
HMGGWRREYPPITSDQQRQRYVEDSKRGAFIYNRLRIVLRTIELELIWLDIILRSLREESEDYMRAAERYNRLKQVKGSAEYKSAKNHAEQLKKKLDHLHKMVEDYLRQKTLE

SEQ ID NO: 6 - protein, 70 aa, Artificial Sequence
  Synthetic peptide
HMTSKQRQVFIANRRKISARTAILELMWQDSERNRRLAQREVNKAPQESKEKLQKTLDQLVADKDAEKLE

SEQ ID NO: 7 - protein, 134 aa, Artificial Sequence
  Synthetic peptide
HMSMQEEDTFRELRIFLRQVTHRLAIREALRVFTKPVDPDEVPDYVTVIEQPMDLSSVISKIDLHKYLTVKDYLRDIDLIMRNALKYNPRASFKNNRIAIAARTLALEAYWIIEMELDRKFEQLAEEIQKSRLE

SEQ ID NO: 8 - protein, 116 aa, Artificial Sequence
  Synthetic peptide
HMINEIKKNAQERMDETVEQLKNELSKVRTGGGGTEERRLELAKQVVFAANRALIRVRTIALEAAWRLLMLGSDKEVNKRDISQALEEIEKLTKVAAKKIKEVLEAKIKELREVLE

SEQ ID NO: 9 - protein, 148 aa, Artificial Sequence
  Synthetic peptide
HMGQRWELALQRFWDYLRWVQTLSEQVQEELLSDKAIEELAALAKETERELRNYIAELSKQLTPVAEETKRQLATTLVFVANRLKITMRTIMLELLRYRIAVNALNGQSTEDLRRNLAENLRKSRDDLLITADKLQRVLAVYQAGALE

SEQ ID NO: 10 - protein, 148 aa, Artificial Sequence
  Synthetic peptide
HMGQRWELALQRFWDYLRWVQTLSEQVQEELLSDKAIEELAALAKETERELRNYIAELSKQLTPVAEETKRQLATTLVEVANRLKETMRTIMLELLRYRIAVNALNGQSTEDLRRNLAENLRKSRDDLLITADKLQRVLAVYQAGALE

SEQ ID NO: 11 - protein, 116 aa, Artificial Sequence
  Synthetic peptide
HMINEIKKNAQERMDETVEQLKNELSKVRTGGGGTEERRLELAKQVVFAANRALIRVRTIALEAAWRLRMLGSDKEVNKRDISQALEEIEKLTKVAAKKIKEVLEAKIKELREVLE

SEQ ID NO: 12 - protein, 116 aa, Artificial Sequence
  Synthetic peptide
HMINEIKKNAQERMDETVEQLKNELSKVRTGGGGTEERRLELAKQVVEAANRALERVRTIALEAAWRLRMLGSDKEVNKRDISQALEEIEKLTKVAAKKIKEVLEAKIKELREVLE

SEQ ID NO: 13 - protein, 117 aa, Artificial Sequence
  Synthetic peptide
MINEIKKNAQERMDETVEQLKNELSKVRTGGGGTEERRLELAKQVVFAANRALIRVRTIALEAAWRLRMLGSDKEVNKRDISQALEEIEKLTKVAAKKIKEVLEAKIKELREVMAVN

SEQ ID NO: 14 - protein, 5 aa, Artificial Sequence
  Synthetic peptide
SGGGG

SEQ ID NO: 15 - protein, 3 aa, Artificial Sequence
  Synthetic peptide
GGS

SEQ ID NO: 16 - protein, 16 aa, Artificial Sequence
  Synthetic peptide
GGGGSLVPRGSGGGGS

SEQ ID NO: 17 - protein, 6 aa, Artificial Sequence
  Synthetic peptide
GSGSGS

SEQ ID NO: 18 - protein, 16 aa, Artificial Sequence
  Synthetic peptide
GSGSGSGSGSGSGSGS

SEQ ID NO: 19 - protein, 11 aa, Artificial Sequence
  Synthetic peptide
GGSGGHMGSGG

SEQ ID NO: 20 - protein, 11 aa, Artificial Sequence
  Synthetic peptide
GGSGGSGGSGG

SEQ ID NO: 21 - protein, 5 aa, Artificial Sequence
  Synthetic peptide
GGSGG

SEQ ID NO: 22 - protein, 8 aa, Artificial Sequence
  Synthetic peptide
GGSGGGGG

SEQ ID NO: 23 - protein, 8 aa, Artificial Sequence
  Synthetic peptide
GSGSGSGS

SEQ ID NO: 24 - protein, 31 aa, Artificial Sequence
  Synthetic peptide
GSGSGSGSGSGSGSGSGSGSGSGSGSGSGSG

SEQ ID NO: 25 - protein, 18 aa, Artificial Sequence
  Synthetic peptide
GGGSEGGGSEGGGSEGGG

SEQ ID NO: 26 - protein, 8 aa, Artificial Sequence
  Synthetic peptide
AAGAATAA

SEQ ID NO: 27 - protein, 5 aa, Artificial Sequence
  Synthetic peptide
GGGGG

SEQ ID NO: 28 - protein, 5 aa, Artificial Sequence
  Synthetic peptide
GGSSG

SEQ ID NO: 29 - protein, 11 aa, Artificial Sequence
  Synthetic peptide
GSGGGTGGGSG

SEQ ID NO: 30 - protein, 2 aa, Artificial Sequence
  Synthetic peptide
GT

SEQ ID NO: 31 - protein, 12 aa, Artificial Sequence
  Synthetic peptide
GSGSGSGSGGSG

SEQ ID NO: 32 - protein, 14 aa, Artificial Sequence
  Synthetic peptide
GSGGSGSGGSGGSG

SEQ ID NO: 33 - protein, 30 aa, Artificial Sequence
  Synthetic peptide
SGGGGSRGGGSGGGGSGGGGSGGGGSGGGG

SEQ ID NO: 34 - protein, 1368 aa, Artificial Sequence
  Synthetic peptide
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 35 - protein, 63 aa, Artificial Sequence
  Synthetic peptide
  MISC_FEATURE (1)..(15): Optional residues
  MISC_FEATURE (32)..(63): Optional residues
YPYDVPDYASLGSGSPKKKRKVEDPKKKRKVDGIGSGSNGSSGSATNFSLLKQAGDVEENPGP

SEQ ID NO: 36 - protein, 236 aa, Artificial Sequence
  Synthetic peptide
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 37 - protein, 1515 aa, Artificial Sequence
  Synthetic peptide
MINEIKKNAQERMDETVEQLKNELSKVRTGGGGTEERRLELAKQVVFAANRALIRVRTIALEAAWRLRMLGSDKEVNKRDISQALEEIEKLTKVAAKKIKEVLEAKIKELREVMAVNSGGGGSRGGGSGGGGSGGGGSGGGGSGGGGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 38 - protein, 1814 aa, Artificial Sequence
  Synthetic peptide
MINEIKKNAQERMDETVEQLKNELSKVRTGGGGTEERRLELAKQVVFAANRALIRVRTIALEAAWRLRMLGSDKEVNKRDISQALEEIEKLTKVAAKKIKEVLEAKIKELREVMAVNSGGGGSRGGGSGGGGSGGGGSGGGGSGGGGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDYPYDVPDYASLGSGSPKKKRKVEDPKKKRKVDGIGSGSNGSSGSATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 39 - protein, 117 aa, Artificial Sequence
  Synthetic peptide
MINEIKKNAQERMDETVEQLKNELSKVRTGGGGTEERRLELAKQVVEAANRALERVRTIALEAAWRLRMLGSDKEVNKRDISQALEEIEKLTKVAAKKIKEVLEAKIKELREVMAVN

SEQ ID NO: 40 - protein, 1388 aa, Streptococcus thermophilus
MTKPYSIGLDIGTNSVGWAVTTDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKAYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKKLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDVPSLEVVKKRKTFWYQLLKSKLISQRKFDNLTKAERGGLSPEDKAGFIQRQLVETRQITKHVARLLDEKFNNKKDENNRAVRTVKIITLKSTLVSQFRKDFELYKVREINDFHHAHDAYLNAVVASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFTVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 41 - protein, 1133 aa, Enterococcus canis
MKQNKELVNIGFDIGIASVGWSVVSKQSGKILETGVSIFPSGTASKNEERRSFRQARRLLRRRKNRISDLKILLEENGFRIAKLNQLVTPYELRVRGLNEQLSKEELSVALLHLVKRRGISYSLEDSEGEGDNQTSYKQSVSINQKLLKEKTPGEIQLERLEKYGKIRGQVKDLQEENAAVLMNVFPNTAYVREAELILLKQKEYYSEITDNFIKEATALISRKREYFVGPGSEKSRTDYGIYRTDGTKLDNLFEILIGKDKIFPNEFRAAGNSYTAQLYNLLNDLNNLKIKTLEDGKLTKDQKLSIIEELKTTTKKVNMMQLIKKIAKAEESDISGYRIDRNDKPEIHSMAIFYKVRKKFLEQEIDINDWPIDFLDILGRVLTLNTENGEIRRSLTELKKDYIFLDETLIELIINSKDSFKLTSNQKWHRFSLKTMQLLIPELLNSSKEQMTILTELGLLHENKQDYSNKTKIDVKNLTENIYNPVVRKSVKQAMDIFNSLFKKYPNIAYLVVEMPRDEAEDEVEQKKQAQKFQKENEAEKEKSLKEFQELAGVSDSQLENQIYKRRKLRMKIRLWYQQLGKCPYSGKTIAAEDLFWTDHLFEIDHVIPLSISYDDGQNNKVLCYSEMNQEKGQKTPYGFMQSGKGQGFSALQAMLKSNSRMSGAKKRNLLFTEDINDIEVRKRFIARNLVDTRYASRIVLNELQQFTRSKQLDTKVTVIRGKFTSKLRETWRLNKSRETHHHHAVDATIIAVSPMLKLWERNAEIIPMKVNENVVDIKTGEILTDKVYQEEMYQLPYASLLEDIAVMENKIKFHHQVDKKMNRKVSDATIYATRSAKVGKDKEPQNYVLGKIKDIYDTKEYENFKKIYDKDKSKFLMQQLDPMTFEKLEKVLKEYPDFEEVQQDNGRVKRIPISPFELYRREKGPITKFAKRNNGPAIKSVKYYDSKMGSAIDITPQTAKNKKVVLQSLKPWRTDVYFNQETKEYEIMGIKYSDMQYLNGNYGITNERYKEIQREEGVADNSEFMMSLYRGDRIKVIDTNSDESVELLFGSRTIPTKKGYVELKPIEKTKFDSKEIVSFYGQVTPNGQFVKKFTRNGYRLLKVNTNILGNPYYISKEGINPRNILDTGFKG

SEQ ID NO: 42 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 43 - protein, 1409 aa, Streptococcus thermophilus
MLFNKCIIISINLDFSNKEKCMTKPYSIGLDIGTNSVGWAVITDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKVYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKNLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDFPSLEVVKKRKTFWYQLLKSKLISQRKFDNLTKAERGGLLPEDKAGFIQRQLVETRQITKHVARLLDEKFNNKKDENNRAVRTVKIITLKSTLVSQFRKDFELYKVREINDFHHAHDAYLNAVIASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFAVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 44 - protein, 1368 aa, Streptococcus pyogenes [sequence omitted]

SEQ ID NO: 45 - protein, 1082 aa, Neisseria meningitidis
MAAFKPNSINYILGLDIGIASVGWAMVEIDEEENPIRLIDLGVRVFERAEVPKTGDSLAMARRLARSVRRLTRRRAHRLLRTRRLLKREGVLQAANFDENGLIKSLPNTPWQLRAAALDRKLTPLEWSAVLLHLIKHRGYLSQRKNEGETADKELGALLKGVAGNAHALQTGDFRTPAELALNKFEKESGHIRNQRSDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTLEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 46 - protein, 1629 aa, Francisella novicida
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 47 - protein, 1395 aa, Treponema denticola
MKKEIKDYFLGLDVGTGSVGWAVTDTDYKLLKANRKDLWGMRCFETAETAEVRRLHRGARRRIERRKKRIKLLQELFSQEIAKTDEGFFQRMKESPFYAEDKTILQENTLFNDKDFADKTYHKAYPTINHLIKAWIENKVKPDPRLLYLACHNIIKKRGHFLFEGDFDSENQFDTSIQALFEYLREDMEVDIDADSQKVKEILKDSSLKNSEKQSRLNKILGLKPSDKQKKAITNLISGNKINFADLYDNPDLKDAEKNSISFSKDDFDALSDDLASILGDSFELLLKAKAVYNCSVLSKVIGDEQYLSFAKVKIYEKHKTDLTKLKNVIKKHFPKDYKKVFGYNKNEKNNNNYSGYVGVCKTKSKKLIINNSVNQEDFYKFLKTILSAKSEIKEVNDILTEIETGTFLPKQISKSNAEIPYQLRKMELEKILSNAEKHFSFLKQKDEKGLSHSEKIIMLLTFKIPYYIGPINDNHKKFFPDRCWVVKKEKSPSGKTTPWNFFDHIDKEKTAEAFITSRTNFCTYLVGESVLPKSSLLYSEYTVLNEINNLQIIIDGKNICDIKLKQKIYEDLFKKYKKITQKQISTFIKHEGICNKTDEVIILGIDKECTSSLKSYIELKNIFGKQVDEISTKNMLEEIIRWATIYDEGEGKTILKTKIKAEYGKYCSDEQIKKILNLKFSGWGRLSRKFLETVTSEMPGFSEPVNIITAMRETQNNLMELLSSEFTFTENIKKINSGFEDAEKQFSYDGLVKPLFLSPSVKKMLWQTLKLVKEISHITQAPPKKIFIEMAKGAELEPARTKTRLKILQDLYNNCKNDADAFSSEIKDLSGKIENEDNLRLRSDKLYLYYTQLGKCMYCGKPIEIGHVFDTSNYDIDHIYPQSKIKDDSISNRVLVCSSCNKNKEDKYPLKSEIQSKQRGFWNFLQRNNFISLEKLNRLTRATPISDDETAKFIARQLVETRQATKVAAKVLEKMFPETKIVYSKAETVSMFRNKFDIVKCREINDFHHAHDAYLNIVVGNVYNTKFTNNPWNFIKEKRDNPKIADTYNYYKVFDYDVKRNNITAWEKGKTIITVKDMLKRNTPIYTRQAACKKGELFNQTIMKKGLGQHPLKKEGPFSNISKYGGYNKVSAAYYTLIEYEEKGNKIRSLETIPLYLVKDIQKDQDVLKSYLTDLLGKKEFKILVPKIKINSLLKINGFPCHITGKTNDSFLLRPAVQFCCSNNEVLYFKKIIRFSEIRSQREKIGKTISPYEDLSFRSYIKENLWKKTKNDEIGEKEFYDLLQKKNLEIYDMLLTKHKDTIYKKRPNSATIDILVKGKEKFKSLIIENQFEVILEILKLFSATRNVSDLQHIGGSKYSGVAKIGNKISSLDNCILIYQSITGIFEKRIDLLKV

SEQ ID NO: 48 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 49 - protein, 1228 aa, Lachnospiraceae sp.
AASKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGAAGYKSLFKKDIIETILPEAADDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNAKTKQALPKFKPLYKQVLSDRESLSFYGEGYTSDEEVLEVFRNTLNKNSEIFSSIKKLEKLFKNFDEYSSAGIFVKNGPAISTISKDIFGEWNLIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFAAAKKNNVFAWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 50 - protein, 1159 aa, Leptotrichia bucallis
MKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLRNIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHRLVGYTSIWERDLRFRLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSEN

SEQ ID NO: 51 - protein, 1389 aa, Leptotrichia shahii
MGNLFGHKRWYEVRDKKDFKIKRKVKVKRNYDGNKYILNINENNNKEKIDNNKFIRKYINYKKNDNILKEFTRKFHAGNILFKLKGKEGIIRIENNDDFLETEEVVLYIEAYGKSEKLKALGITKKKIIDEAIRQGITKDDKKIEIKRQENEEEIEIDIRDEYTNKTLNDCSIILRIIENDELETKKSIYEIFKNINMSLYKIIEKIIENETEKVFENRYYEEHLREKLLKDDKIDVILTNFMEIREKIKSNLEILGFVKFYLNVGGDKKKSKNKKMLVEKILNINVDLTVEDIADFVIKELEFWNITKRIEKVKKVNNEFLEKRRNRTYIKSYVLLDKHEKFKIERENKKDKIVKFFVENIKNNSIKEKIEKILAEFKIDELIKKLEKELKKGNCDTEIFGIFKKHYKVNFDSKKFSKKSDEEKELYKIIYRYLKGRIEKILVNEQKVRLKKMEKIEIEKILNESILSEKILKRVKQYTLEHIMYLGKLRHNDIDMTTVNTDDFSRLHAKEELDLELITFFASTNMELNKIFSRENINNDENIDFFGGDREKNYVLDKKILNSKIKIIRDLDFIDNKNNITNNFIRKFTKIGTNERNRILHAISKERDLQGTQDDYNKVINIIQNLKISDEEVSKALNLDVVFKDKKNIITKINDIKISEENNNDIKYLPSFSKVLPEILNLYRNNPKNEPFDTIETEKIVLNALIYVNKELYKKLILEDDLEENESKNIFLQELKKTLGNIDEIDENIIENYYKNAQISASKGNNKAIKKYQKKVIECYIGYLRKNYEELFDFSDFKMNIQEIKKQIKDINDNKTYERITVKTSDKTIVINDDFEYIISIFALLNSNAVINKIRNRFFATSVWLNTSEYQNIIDILDEIMQLNTLRNECITENWNLNLEEFIQKMKEIEKDFDDFKIQTKKEIFNNYYEDIKNNILTEFKDDINGCDVLEKKLEKIVIFDDETKFEIDKKSNILQDEQRKLSNINKKDLKKKVDQYIKDKDQEIKSKILCRIIFNSDFLKKYKKEIDNLIEDMESENENKFQEIYYPKERKNELYIYKKNLFLNIGNPNFDKIYGLISNDIKMADAKFLFNIDGKNIRKNKISEIDAILKNLNDKLNGYSKEYKEKYIKKLKENDDFFAKNIQNKNYKSFEKDYNRVSEYKKIRDLVEFNYLNKIESYLIDINWKLAIQMARFERDMHYIVNGLRELGIIKLSGYNTGISRAYPKRNGSDGFYTTTAYYKFFDEESYKKFEKICYGFGIDLSENSEINKPENESIRNYISHFYIVRNPFADYSIAEQIDRVSNLLSYSTRYNNSTYASVFEVFKKDVNLDYDELKKKFKLIGNNDILERLMKPKKVSVLELESYNSDYIKNLIIELLTKIENTNDTL

SEQ ID NO: 52 - protein, 984 aa, Campylobacter jejuni
MARILAFDIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 53 - protein, 1368 aa, Staphylococcus aureus; identical to SEQ ID NO: 34

SEQ ID NO: 54 - protein, 1164 aa, Xanthomonas euvesicatoria
MDPIRSRTPSPARELLPGPQPDGVQPTADRGVSPPAGGPLDGLPARRTMSRTRLPSPPAPSPAFSAGSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASNGGGKQALETVQRLLPVLCQAHGLTPQQVVAIASNSGGKQALETVQRLLPVLCQAHGLTPEQVVAIASNGGGKQALETVQRLLPVLCQAHGLTPEQVVAIASNIGGKQALETVQALLPVLCQAHGLTPEQVVAIASNIGGKQALETVQALLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASNSGGKQALETVQALLPVLCQAHGLTPEQVVAIASNSGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGRPALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRASSRKRSRSDRAVTGPSAQQSFEVRVPEQRDALHLPLSWRVKRPRTSIGGGLPDPGTPTAADLAASSTVMREQDEDPFAGAADDFPAFNEEELAWLMELLPQ

SEQ ID NO: 55 - protein, 1373 aa, Xanthomonas oryzae
MDPIRSRTPSPARELLPGPQPDRVQPTADRGGAPPAGGPLDGLPARRTMSRTRLPSPPAPSPAFSAGSFSDLLRQFDPSLLDTSLLDSMPAVGTPHTAAAPAECDEVQSGLRAADDPPPTVRVAVTAARPPRAKPAPRRRAAQPSDASPAAQVDLRTLGYSQQQQEKIKPKVGSTVAQHHEALVGHGFTHAHIVALSRHPAALGTVAVKYQDMIAALPEATHEDIVGVGKQWSGARALEALLTVAGELRGPPLQLDTGQLVKIAKRGGVTAVEAVHASRNALTGAPLNLTPAQVVAIASNNGGKQALETVQRLLPVLCQAHGLTPAQVVAIASHDGGKQALETMQRLLPVLCQAHGLPPDQVVAIASNIGGKQALETVQRLLPVLCQAHGLTPDQVVAIASHGGGKQALETVQRLLPVLCQAHGLTPDQVVAIASHDGGKQALETVQRLLPVLCQAHGLTPDQVVAIASNGGGKQALETVQRLLPVLCQAHGLTPDQVVAIASNGGKQALETVQRLLPVLCQAHGLTPDQVVAIASHDGGKQALETVQRLLPVLCQTHGLTPAQVVAIASHDGGKQALETVQQLLPVLCQAHGLTPDQVVAIASNIGGKQALATVQRLLPVLCQAHGLTPDQVVAIASNGGGKQALETVQRLLPVLCQAHGLTPDQVVAIASNGGGKQALETVQRLLPVLCQAHGLTQVQVVAIASNIGGKQALETVQRLLPVLCQAHGLTPAQVVAIASHDGGKQALETVQRLLPVLCQAHGLTPDQVVAIASNGGGKQALETVQRLLPVLCQAHGLTQEQVVAIASNNGGKQALETVQRLLPVLCQAHGLTPDQVVAIASNGGGKQALETVQRLLPVLCQAHGLTPAQVVAIASNIGGKQALETVQRLLPVLCQDHGLTLAQVVAIASNIGGKQALETVQRLLPVLCQAHGLTQDQVVAIASNIGGKQALETVQRLLPVLCQDHGLTPDQVVAIASNIGGKQALETVQRLLPVLCQDHGLTLDQVVAIASNGGKQALETVQRLLPVLCQDHGLTPDQVVAIASNSGGKQALETVQRLLPVLCQDHGLTPNQVVAIASNGGKQALESIVAQLSRPDPALAALTNDHLVALACLGGRPAMDAVKKGLPHAPELIRRVNRRIGERTSHRVADYAQVVRVLEFFQCHSHPAYAFDEAMTQFGMSRNGLVQLFRRVGVTELEARGGTLPPASQRWDRILQASGMKRAKPSPTSAQTPDQASLHAFADSLERDLDAPSPMHEGDQTGASSRKRSRSDRAVTGPSAQHSFEVRVPEQRDALHLPLSWRVKRPRTRIGGGLPDPGTPIAADLAASSTVMWEQDAAPFAGAADDFPAFNEEELAWLMELLPQSGSVGGTI

SEQ ID NO: 56 - protein, 287 aa, Gremmeniella abietina
INPWFLTGFIDGEGCFRISVTKINRAIDWRVQLFFQINLHEKDRALLESIKDYLKVGKIHISGKNLVQYRIQTFDELTILIKHLKEYPLVSKKRADFELFNTAHKLIKNNEHLNKEGINKLVSLKASLNLGLSESLKLAFPNVISATRLTDFTVNIPDPHWLSGFASAEGCFMVGIAKSSASSTGYQVYLTFILTQHVRDENLMKCLVDYFNWGRLARKRNVYEYQVSKFSDVEKLLSFFDKYPILGEKAKDLQDFCSVSDLMKSKTHLTEEGVAKIRKIKEGMNRG

SEQ ID NO: 57 - protein, 404 aa, Arabidopsis thaliana
MRTPMSDTQHVQSSLVSIRSSDKIEDAFRKMKVNETGVEELNPYPDRPGERDCQFYLRTGLCGYGSSCRYNHPTHLPQDVAYYKEELPERIGQPDCEYFLKTGACKYGPTCKYHHPKDRNGAQPVMFNVIGLPMRLGEKPCPYYLRTGTCRFGVACKFHHPQPDNGHSTAYGMSSFPAADLRYASGLTMMSTYGTLPRPQVPQSYVPILVSPSQGFLPPQGWAPYMAASNSMYNVKNQPYYSGSSASMAMAVALNRGLSESSDQPECRFFMNTGTCKYGDDCKYSHPGVRISQPPPSLINPFVLPARPGQPACGNFRSYGFCKFGPNCKFDHPMLPYPGLTMATSLPTPFASPVTTHQRISPTPNRSDSKSLSNGKPDVKKESSETEKPDNGEVQDLSEDASSP

SEQ ID NO: 58 - protein, 1815 aa, Artificial Sequence
  Synthetic peptide
MINEIKKNAQERMDETVEQLKNELSKVRTGGGGTEERRLELAKQVVFAANRALIRVRTIALEAAWRLRMLGSDKEVNKRDISQALEEIEKLTKVAAKKIKEVLEAKIKELREVMAVNSGGGGSRGGGSGGGGSGGGGSGGGGSGGGGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGIGSGSNGSSGSATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 59 - protein, 1815 aa, Artificial Sequence
  Synthetic peptide
MINEIKKNAQERMDETVEQLKNELSKVRTGGGGTEERRLELAKQVVEAANRALERVRTIALEAAWRLRMLGSDKEVNKRDISQALEEIEKLTKVAAKKIKEVLEAKIKELREVMAVNSGGGGSRGGGSGGGGSGGGGSGGGGSGGGGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGIGSGSNGSSGSATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 60 - protein, 8 aa, Artificial Sequence
  Synthetic peptide
  MISC_FEATURE (2)..(2): Xaa can be any amino acid
  MISC_FEATURE (6)..(6): Xaa can be any amino acid
  MISC_FEATURE (7)..(7): Xaa can be any amino acid
FXANRXXI

SEQ ID NO: 61 - DNA, 31 bp, Artificial Sequence
  Input position sequence position 79 to 110
tatatgacggactgagcgttaacgtaacatt

SEQ ID NO: 62 - DNA, 33 bp, Artificial Sequence
  Input position sequence position 79 to 112
tatatgacggactgagcgttaacgtaacatttt

SEQ ID NO: 63 - DNA, 41 bp, Artificial Sequence
  Input position sequence position 79 to 119
tatatgacggactgagcgttaacgtaacattttgcaactcc